(19) United States
(12) Patent Application Publication
(10) Pub. No.: US 2025/0049846 A1
(43) Pub. Date: Feb. 13, 2025

(54) METHODS AND COMPOSITIONS FOR TREATING CANCER WITH IMMUNE CELLS

(71) Applicant: Palleon Pharmaceuticals Inc., Waltham, MA (US)

(72) Inventors: Ahmed Alkhateeb, Cambridge, MA (US); Karl D. Normington, Prides Crossing, MA (US); Li Peng, Lexington, MA (US); James W. Broderick, Wellesley, MA (US); Zakir B. Siddiquee, Cambridge, MA (US); Jenny Che, Ashland, MA (US); Weiguo Yao, Auburndale, MA (US); Abhishek Das, Wayland, MA (US)

(21) Appl. No.: 17/417,513

(22) PCT Filed: Jan. 3, 2020
(86) PCT No.: PCT/US2020/012240

§ 371 Date: Jun. 23, 2021

Related U.S. Application Data
(60) Provisional application No. 62/787,935, filed on Jan. 3, 2019.

(51) Int. Cl. A61K 35/17; A61K 39/00; A61P 35/00; C12N 5/0783; C12N 9/24

(52) U.S. Cl. CPC A61K 35/17; A61K 39/4611; A61K 39/4631; A61K 39/464412; A61K 39/464468; A61P 35/00; C12N 5/0636; C12N 9/2402; A61K 2239/13; C12N 2510/00; C12Y 302/01018

(57) ABSTRACT

The invention provides compositions and methods for treating a patient with cancer with an immune cell, e.g., a T-cell, e.g., an engineered T-cell, with increased sialidase activity. The invention also provides compositions and methods for treating a patient with cancer with an immune cell, e.g., a T-cell, e.g., an engineered T-cell, in combination with a sialidase. The invention also provides compositions and methods for treating a patient with cancer with an immune cell, e.g., a T-cell, e.g., an engineered T-cell, pretreated with a sialidase.

### CROSS REFERENCE TO RELATED APPLICATION

This application is a § 371 National Stage of International (PCT) Patent Application Serial No. PCT/US2020/012240, filed on Jan. 3, 2020, which claims the benefit of, and priority to, U.S. Provisional Patent Application No. 62/787,935, filed on Jan. 3, 2019, the disclosures of each of which are hereby incorporated by reference herein in their entirety for all purposes.

### SEQUENCE LISTING

The instant application contains a Sequence Listing which has been submitted electronically in ASCII format and is hereby incorporated by reference in its entirety. Said ASCII copy, created on Aug. 19, 2021, is named PAL-010WOUS_SL.txt and is 701,920 bytes in size.

### FIELD OF THE INVENTION

The invention relates generally to compositions and methods for treating cancer in a subject, and, more particularly, the invention relates to immune cells with increased sialidase activity or pharmaceutical compositions containing such immune cells and their use in the treatment of cancer.

### BACKGROUND

According to the American Cancer Society, more than one million people in the United States are diagnosed with cancer each year. Cancer is a disease that results from uncontrolled proliferation of cells that were once subject to natural control mechanisms but have been transformed into cancerous cells that continue to proliferate in an uncontrolled manner.

A growing body of evidence supports roles for glycans, and sialoglycans in particular, at various pathophysiological steps of tumor progression. Glycans regulate tumor proliferation, invasion, hematogenous metastasis and angiogenesis (Fuster et al. (2005) NAT. REV. CANCER 5(7): 526-42). The sialylation of cell surface glycoconjugates is frequently altered in cancers, resulting in the expression of sialylated tumor-associated carbohydrate antigens. The expression of sialylated glycans by tumor cells is often associated with increased aggressiveness and metastatic potential of a tumor.

Chimeric antigen receptors (CARs) are synthetic receptors that retarget immune cells, e.g., T cells, to tumor surface antigens (Sadelain et al. (2003), NAT. REV. CANCER. 3(1):35-45, Sadelain et al. (2013) CANCER DISCOVERY 3(4):388-398). CARs provide both antigen binding and immune cell activation functions. Initially, CARs contained an antibody-based tumor-binding element, such as a single chain Fv (scFv), that is responsible for antigen recognition linked to either CD3zeta or Fc receptor signaling domains, which trigger T-cell activation. Later CAR constructs included additional activating and costimulatory signaling domains which have led to encouraging results in patients with chemorefractory B-cell malignancies (Brentjens et al. (2013) SCI. TRANS. MED. 5(177): 177ra38, Brentjens et al. (2011) BLOOD 118(18): 4817-4828, Davila et al. (2014) SCI. TRANS. MED. 6(224): 224ra25, Grupp et al. (2013) N. ENGL. J. MED. 368(16): 1509-1518, Kalos et al. (2011) SCI. TRANS. MED. 3(95): 95ra73).

Despite the significant advances being made in cancer treatment and management, there is still an ongoing need for new and effective therapies for treating and managing cancer.

### SUMMARY OF THE INVENTION

The present invention provides methods and compositions for treating cancer in a subject. The invention is based, in part, upon the discovery that an anti-cancer treatment using an immune cell, e.g., a T-cell, e.g., a CAR T-cell, can be enhanced when the activity of a sialidase is introduced into, up regulated, or otherwise increased in the immune cell. Additionally, an anti-cancer treatment using an immune cell, e.g., a T-cell, e.g., a CAR T-cell, can be enhanced when the immune cell is administered in combination with a sialidase, or is pretreated with a sialidase before administration to a subject. It is contemplated that the sialidase removes sialic acid and/or sialic acid containing molecules from the surface of cancer cells, immune cells, and/or other molecules in the tumor microenvironment, thereby reducing immune inhibition mediated by the sialic acid and/or sialic acid containing molecules.

Accordingly, in one aspect, the invention provides an isolated immune cell modified to have increased sialidase activity relative to a similar or identical cell that has not been modified. In certain embodiments, the immune cell comprises an exogenous nucleotide sequence encoding a sialidase.

In certain embodiments, the sialidase is a eukaryotic sialidase, e.g., a mammalian sialidase, e.g., a human sialidase, e.g., Neu1, Neu2, Neu3, or Neu4. In certain embodiments, the sialidase comprises an amino acid sequence selected from SEQ ID NO: 1, SEQ ID NO: 2, SEQ ID NO: 3, SEQ ID NO: 4, SEQ ID NO: 5, and SEQ ID NO: 6.

In certain embodiments, the sialidase is a prokaryotic sialidase, e.g., a Salmonella typhimurium sialidase or a Vibrio cholera sialidase. In certain embodiments, the sialidase comprises an amino acid sequence selected from SEQ ID NO: 7 and SEQ ID NO: 8.

In certain embodiments, the sialidase is a wild-type sialidase. In certain embodiments, the sialidase is a mutant sialidase, e.g., the sialidase comprises an amino acid sequence selected from SEQ ID NO: 36, SEQ ID NO: 37, SEQ ID NO: 38, SEQ ID NO: 39, SEQ ID NO: 63, SEQ ID NO: 64, SEQ ID NO: 65, SEQ ID NO: 66, SEQ ID NO: 83, SEQ ID NO: 84, SEQ ID NO: 85, SEQ ID NO: 86, SEQ ID NO: 87, SEQ ID NO: 88, and SEQ ID NO: 89. In certain embodiments, the sialidase can cleave α2,3, α2,6, and/or α2,8 linkages.

In certain embodiments, the isolated immune cell is selected from a T-cell, a Tumor Infiltrating Lymphocyte (TIL), and a natural killer (NK) cell.

In certain embodiments, the isolated immune cell is engineered to express a chimeric antigen receptor (CAR), e.g., the isolated immune cell comprises an exogenous nucleotide sequence encoding a CAR. In certain embodiments, the CAR binds a cancer antigen, for example, a cancer antigen selected from mesothelin, prostate specific membrane antigen (PSMA), prostate stem cell antigen (PCSA), carbonic anhydrase IX (CAIX), carcinoembryonic antigen (CEA), CD5, CD7, CD10, CD19, CD20, CD22, CD30, CD33, CD34, CD38, CD41, CD44, CD47, CD49f, CD56, CD74, CD123, CD133, CD138, epithelial glycoprotein2 (EGP 2), epithelial glycoprotein-40 (EGP-40), epithelial cell adhesion molecule (EpCAM), folate-binding protein (FBP), fetal acetylcholine receptor (AChR), folate receptor-α and β (FRa and β), Ganglioside G2 (GD2), Ganglioside G3 (GD3), an Epidermal Growth Factor Receptor (EGFR), Epidermal Growth Factor Receptor 2 (HER-2/ERB2), Epidermal Growth Factor Receptor vIII (EGFRvIII), ERB3, ERB4, human telomerase reverse transcriptase (hTERT), Interleukin-13 receptor subunit alpha-2 (IL-13Ra2), K-light chain, kinase insert domain receptor (KDR), Lewis A (CA19.9), Lewis Y (LeY), LI cell adhesion molecule (LICAM), melanoma-associated antigen 1 (melanoma antigen family A1, MAGE-A1), Mucin 16 (MUC-16), Mucin 1 (MUC-1), KG2D ligands, cancer-testis antigen NY-ESO-1, oncofetal antigen (h5T4), tumor-associated glycoprotein 72 (TAG-72), vascular endothelial growth factor R2 (VEGF-R2), Wilms tumor protein (WT-1), type 1 tyrosine-protein kinase transmembrane receptor (ROR1), B7-H3 (CD276), B7-H6 (Nkp30), Chondroitin sulfate proteoglycan-4 (CSPG4), DNAX Accessory Molecule (DNAM-1), Ephrin type A Receptor 2 (EpHA2), Fibroblast Associated Protein (FAP), Gp100/HLA-A2, Glypican 3 (GPC3), HA-IH, HERK-V, IL-1 IRa, Latent Membrane Protein 1 (LMP1), Neural cell-adhesion molecule (N-CAM/CD56), programmed cell death receptor ligand 1 (PD-L1), B Cell Maturation Antigen (BCMA), and Trail Receptor (TRAIL R).

In another aspect, the invention also provides a pharmaceutical composition comprising any of the foregoing isolated immune cells and a pharmaceutically acceptable carrier or diluent.

In another aspect, the invention provides a pharmaceutical composition comprising: (a) an isolated immune cell; (b) a sialidase; and (c) a pharmaceutically acceptable carrier or diluent.

In another aspect, the invention provides a pharmaceutical composition comprising: (a) an isolated immune cell pretreated with a sialidase; and (b) a pharmaceutically acceptable carrier or diluent.

In certain embodiments, the sialidase is a eukaryotic sialidase, e.g., a mammalian sialidase, e.g., a human sialidase, e.g., Neu1, Neu2, Neu3, or Neu4. In certain embodiments, the sialidase comprises an amino acid sequence selected from SEQ ID NO: 1, SEQ ID NO: 2, SEQ ID NO: 3, SEQ ID NO: 4, SEQ ID NO: 5, and SEQ ID NO: 6.

In certain embodiments, the sialidase is a prokaryotic sialidase, e.g., a Salmonella typhimurium sialidase or a Vibrio cholera sialidase. In certain embodiments, the sialidase comprises an amino acid sequence selected from SEQ ID NO: 7 and SEQ ID NO: 8.

In certain embodiments, the sialidase is a wild-type sialidase. In certain embodiments, the sialidase is a mutant sialidase, e.g., the sialidase comprises an amino acid sequence selected from SEQ ID NO: 36, SEQ ID NO: 37, SEQ ID NO: 38, SEQ ID NO: 39, SEQ ID NO: 63, SEQ ID NO: 64, SEQ ID NO: 65, SEQ ID NO: 66, SEQ ID NO: 83, SEQ ID NO: 84, SEQ ID NO: 85, SEQ ID NO: 86, SEQ ID NO: 87, SEQ ID NO: 88, and SEQ ID NO: 89. In certain embodiments, the sialidase can cleave α2,3, α2,6, and/or α2,8 linkages.

In certain embodiments, the isolated immune cell is selected from a T-cell, a Tumor Infiltrating Lymphocyte (TIL), and a natural killer (NK) cell.

In certain embodiments, the isolated immune cell is engineered to express a chimeric antigen receptor (CAR), e.g., the isolated immune cell comprises an exogenous nucleotide sequence encoding a CAR. In certain embodiments, the CAR binds a cancer antigen, for example, a cancer antigen selected from mesothelin, prostate specific membrane antigen (PSMA), prostate stem cell antigen (PCSA), carbonic anhydrase IX (CAIX), carcinoembryonic antigen (CEA), CD5, CD7, CD10, CD19, CD20, CD22, CD30, CD33, CD34, CD38, CD41, CD44, CD47, CD49f, CD56, CD74, CD123, CD133, CD138, epithelial glycoprotein2 (EGP 2), epithelial glycoprotein-40 (EGP-40), epithelial cell adhesion molecule (EpCAM), folate-binding protein (FBP), fetal acetylcholine receptor (AChR), folate receptor-α and β (FRa and β), Ganglioside G2 (GD2), Ganglioside G3 (GD3), an Epidermal Growth Factor Receptor (EGFR), Epidermal Growth Factor Receptor 2 (HER-2/ERB2), Epidermal Growth Factor Receptor vIII (EGFRvIII), ERB3, ERB4, human telomerase reverse transcriptase (hTERT), Interleukin-13 receptor subunit alpha-2 (IL-13Ra2), K-light chain, kinase insert domain receptor (KDR), Lewis A (CA19.9), Lewis Y (LeY), LI cell adhesion molecule (LICAM), melanoma-associated antigen 1 (melanoma antigen family A1, MAGE-A1), Mucin 16 (MUC-16), Mucin 1 (MUC-1), KG2D ligands, cancer-testis antigen NY-ESO-1, oncofetal antigen (h5T4), tumor-associated glycoprotein 72 (TAG-72), vascular endothelial growth factor R2 (VEGF-R2), Wilms tumor protein (WT-1), type 1 tyrosine-protein kinase transmembrane receptor (ROR1), B7-H3 (CD276), B7-H6 (Nkp30), Chondroitin sulfate proteoglycan-4 (CSPG4), DNAX Accessory Molecule (DNAM-1), Ephrin type A Receptor 2 (EpHA2), Fibroblast Associated Protein (FAP), Gp100/HLA-A2, Glypican 3 (GPC3), HA-IH, HERK-V, IL-1 IRa, Latent Membrane Protein 1 (LMP1), Neural cell-adhesion molecule (N-CAM/CD56), programmed cell death receptor ligand 1 (PD-L1), B Cell Maturation Antigen (BCMA), and Trail Receptor (TRAIL R).

In another aspect, the invention provides a method of inhibiting tumor growth in a subject in need thereof, the method comprising administering to the subject an effective amount of any of the foregoing isolated immune cells or pharmaceutical compositions, thereby to inhibit growth of the tumor. In another aspect, the invention provides a method of treating cancer in a subject in need thereof, the method comprising administering to the subject an effective amount of any of the foregoing isolated immune cells or pharmaceutical compositions, thereby to treat the cancer in the subject.

In another aspect, the invention provides a method of treating cancer in a subject in need thereof, the method comprising administering to the subject an isolated immune cell and a sialidase, thereby to treat the cancer in the subject. The isolated immune cell and the sialidase may, e.g., be administered separately or in combination. The isolated immune cell and the sialidase may, e.g., be administered at the same time or at different times.

In another aspect, the invention provides a method of treating cancer in a subject in need thereof, the method comprising administering an isolated, sialidase treated immune cell to the subject. The isolated immune cell may, e.g., be substantially sialidase free.

In another aspect, the invention provides a method of treating cancer in a subject in need thereof, the method comprising: (a) pretreating an isolated immune cell with a sialidase; and (b) administering the isolated immune cell to the subject. The isolated immune cell may, e.g., be purified from the sialidase prior to administration to the subject.

In certain embodiments, the sialidase is a eukaryotic sialidase, e.g., a mammalian sialidase, e.g., a human sialidase, e.g., Neu1, Neu2, Neu3, or Neu4. In certain embodiments, the sialidase comprises an amino acid sequence selected from SEQ ID NO: 1, SEQ ID NO: 2, SEQ ID NO: 3, SEQ ID NO: 4, SEQ ID NO: 5, and SEQ ID NO: 6.

In certain embodiments, the sialidase is a prokaryotic sialidase, e.g., a Salmonella typhimurium sialidase or a Vibrio cholera sialidase. In certain embodiments, the sialidase comprises an amino acid sequence selected from SEQ ID NO: 7 and SEQ ID NO: 8.

In certain embodiments, the sialidase is a wild-type sialidase. In certain embodiments, the sialidase is a mutant sialidase, e.g., the sialidase comprises an amino acid sequence selected from SEQ ID NO: 36, SEQ ID NO: 37, SEQ ID NO: 38, SEQ ID NO: 39, SEQ ID NO: 63, SEQ ID NO: 64, SEQ ID NO: 65, SEQ ID NO: 66, SEQ ID NO: 83, SEQ ID NO: 84, SEQ ID NO: 85, SEQ ID NO: 86, SEQ ID NO: 87, SEQ ID NO: 88, and SEQ ID NO: 89. In certain embodiments, the sialidase can cleave α2,3, α2,6, and/or α2,8 linkages.

In certain embodiments, the isolated immune cell is selected from a T-cell, a Tumor Infiltrating Lymphocyte (TIL), and a natural killer (NK) cell.

In certain embodiments, the isolated immune cell is engineered to express a chimeric antigen receptor (CAR), e.g., the isolated immune cell comprises an exogenous nucleotide sequence encoding a CAR. In certain embodiments, the CAR binds a cancer antigen, for example, a cancer antigen selected from mesothelin, prostate specific membrane antigen (PSMA), prostate stem cell antigen (PCSA), carbonic anhydrase IX (CAIX), carcinoembryonic antigen (CEA), CD5, CD7, CD10, CD19, CD20, CD22, CD30, CD33, CD34, CD38, CD41, CD44, CD47, CD49f, CD56, CD74, CD123, CD133, CD138, epithelial glycoprotein2 (EGP 2), epithelial glycoprotein-40 (EGP-40), epithelial cell adhesion molecule (EpCAM), folate-binding protein (FBP), fetal acetylcholine receptor (AChR), folate receptor-α and β (FRa and β), Ganglioside G2 (GD2), Ganglioside G3 (GD3), an Epidermal Growth Factor Receptor (EGFR), Epidermal Growth Factor Receptor 2 (HER-2/ERB2), Epidermal Growth Factor Receptor vIII (EGFRvIII), ERB3, ERB4, human telomerase reverse transcriptase (hTERT), Interleukin-13 receptor subunit alpha-2 (IL-13Ra2), K-light chain, kinase insert domain receptor (KDR), Lewis A (CA19.9), Lewis Y (LeY), LI cell adhesion molecule (LICAM), melanoma-associated antigen 1 (melanoma antigen family A1, MAGE-A1), Mucin 16 (MUC-16), Mucin 1 (MUC-1), KG2D ligands, cancer-testis antigen NY-ESO-1, oncofetal antigen (h5T4), tumor-associated glycoprotein 72 (TAG-72), vascular endothelial growth factor R2 (VEGF-R2), Wilms tumor protein (WT-1), type 1 tyrosine-protein kinase transmembrane receptor (ROR1), B7-H3 (CD276), B7-H6 (Nkp30), Chondroitin sulfate proteoglycan-4 (CSPG4), DNAX Accessory Molecule (DNAM-1), Ephrin type A Receptor 2 (EpHA2), Fibroblast Associated Protein (FAP), Gp100/HLA-A2, Glypican 3 (GPC3), HA-IH, HERK-V, IL-1 IRa, Latent Membrane Protein 1 (LMP1), Neural cell-adhesion molecule (N-CAM/CD56), programmed cell death receptor ligand 1 (PD-L1), B Cell Maturation Antigen (BCMA), and Trail Receptor (TRAIL R).

In certain embodiments of any of the foregoing methods, the cancer is an adenocarcinoma, a metastatic cancer and/or is a refractory cancer. In certain embodiments of any of the foregoing methods, the cancer is a breast, colon or colorectal, lung, ovarian, pancreatic, prostate, cervical, endometrial, head and neck, liver, renal, skin, stomach, testicular, thyroid or urothelial cancer. In certain embodiments of any of the foregoing methods, the cancer is an epithelial cancer, e.g., an endometrial cancer, ovarian cancer, cervical cancer, vulvar cancer, uterine cancer, fallopian tube cancer, breast cancer, prostate cancer, lung cancer, pancreatic cancer, urinary cancer, bladder cancer, head and neck cancer, oral cancer or liver cancer.

In certain embodiments of any of the foregoing methods, the cancer is a hematologic cancer, e.g., a leukemia, lymphoma, or multiple myeloma, e.g., Chronic lymphocytic leukemia (CLL), Acute myeloid leukemia (AML), Chronic myelogenous leukemia (CML), Non-Hodgkin lymphoma (NHL), Burkitt lymphoma, Chronic myeloid monocytic leukemia (CMML), Eosinphilia, Essential thrombocytosis, Hairy cell leukemia, and NK cell lymphoma.

In certain embodiments of any of the foregoing methods, the method further comprises administering an IDO antagonist, or an immune checkpoint antagonist, e.g., a PD-1 antagonist, PD-L1 antagonist, CTLA-4 antagonist, adenosine A2A receptor antagonist, B7-H3 antagonist, B7-H4 antagonist, BTLA antagonist, KIR antagonist, LAG3 antagonist, TIM-3 antagonist, VISTA antagonist or TIGIT antagonist. In certain embodiments of any of the foregoing methods, the subject is a human.

These and other aspects and features of the invention are described in the following detailed description and claims.

### DESCRIPTION OF THE DRAWINGS

The invention can be more completely understood with reference to the following drawings.

FIGS. 1A-C are schematic representations of three antibody sialidase conjugates (ASCs). The first type of ASC, referred to as “Raptor,” includes an antibody (with two heavy chains and two light chains) with a sialidase fused at the C-terminus of each heavy chain of the antibody (FIG. 1A). The second type of ASC, referred to as “Janus,” contains one antibody arm (with one heavy chain and one light chain), and one sialidase-Fc fusion with a sialidase fused at the N-terminus of the Fc. Each Fc domain polypeptide in the Janus ASC may contain either the “knob” (T366Y) or “hole” (Y407T) mutation for heterodimerization (residue numbers according to EU numbering, Kabat, E. A., et al. (1991) supra) (FIG. 1B). The third type of ASC, referred to as “Lobster,” contains two Fc domain polypeptides each with a sialidase fused at the N-terminus of the Fc and a scFv fused at the C-terminus of the Fc (FIG. 1C).

FIG. 2 depicts an SDS-PAGE gel showing recombinant human Neu1, Neu2, Neu3, and Salmonella typhimurium (St-sialidase) under non-reducing and reducing conditions. Monomer and dimer species are indicated.

FIG. 3 is a bar graph showing the enzymatic activity of recombinant human Neu1, Neu2, and Neu3.

FIG. 4 is a line graph showing enzymatic activity as a function of substrate concentration for recombinant human Neu2 and Neu3 at the indicated pH.

FIG. 5 depicts the degree of sialic acid removal from three human cell lines by neuraminidase constructs of the current invention. Specifically, Raji Burkitt lymphoma cells, Ramos lymphoma cells and SKOV-3 ovarian cells were treated for 16 hours using either a mutant neuraminidase lacking enzymatic activity (LOF), neuraminidase construct #1or untreated cells (No Tx). Cells were then stained with PNA, a lectin that binds to terminal galactose residues. An increase in PNA staining is indicative of the removal of terminal sialic acids by the neuraminidase and exposure of the underlying galactose.

FIGS. 6A and 6B depicts the degree of sialic acid removal from the CD19 positive Raji cells (FIG. 6A) and CD3 positive CAR-T cells (FIG. 6B) following 16 hours cotreatment with neuraminidase construct #1. Specifically, both target and effector cell were analyzed for PNA staining following 16 hours with either a mutant neuraminidase lacking enzymatic activity (LOF), neuraminidase construct #1 or untreated cells (No Tx).

FIGS. 7A and 7B depict the degree of sialic acid removal from the CD19 positive Ramos cells (FIG. 7A) and CD3 positive CAR-T cells (FIG. 7B) following 16 hours cotreatment with neuraminidase construct #1. Specifically, both target and effector cells were analyzed for PNA staining following 16 hours with either a mutant neuraminidase lacking enzymatic activity (LOF), neuraminidase construct #1 or untreated cells (No Tx).

FIGS. 8A, B and C depict the analysis of secreted cytokines from the 16 hour time point. Specifically, following incubation of the CAR-T cells, Raji target cells and neuraminidase construct #1 for 16 hours, cells were removed, and conditioned media analyzed for IFN-gamma (FIG. 8A), IL-2 (FIG. 8B) and TNF-alpha (FIG. 8C) by flow multiplex (LegendPlex). In each panel, the No Tx, LOF and neuraminidase construct #1 (respectively) analysis in quadruplicate assays at the indicated E:T ratio is shown.

FIG. 9 depicts the analysis of Siglec7 and Siglec9 expression on CAR-T cells as compared to peripheral blood mononuclear cells (PBMCs) as a positive control. Specifically, CAR-T cells prepared from two independent donors or PBMCs from a healthy donor were stained for Siglec9 expression (left panels) or for Siglec7 expression (right panels) and compared to isotype staining.

### DETAILED DESCRIPTION

The present invention provides methods and compositions for treating cancer in a subject. The invention is based, in part, upon the discovery that an anti-cancer treatment using an immune cell, e.g., a T-cell, e.g., a CAR T-cell, can be enhanced when the activity of a sialidase is introduced into, up regulated, or otherwise increased in the immune cell. Additionally, an anti-cancer treatment using an immune cell, e.g., a T-cell, e.g., a CAR T-cell, can be enhanced when the immune cell is administered in combination with a sialidase, or is pretreated with a sialidase before administration to a subject. It is contemplated that the sialidase removes sialic acid and/or sialic acid containing molecules from the surface of cancer cells, immune cells, and/or other molecules in the tumor microenvironment, thereby reducing immune inhibition mediated by the sialic acid and/or sialic acid containing molecules.

Accordingly, in one aspect, the invention provides an isolated immune cell modified to have increased sialidase activity relative to a similar or identical cell that has not been modified. In certain embodiments, the immune cell comprises an exogenous nucleotide sequence encoding a sialidase. In another aspect, the invention provides a pharmaceutical composition comprising an isolated immune cell, e.g., a T-cell, modified to have up regulated or increased functional activity associated with or modulated by a sialidase. In another aspect, the invention provides a pharmaceutical composition comprising an isolated immune cell, e.g., a T-cell, and a sialidase. In another aspect, the invention provides a pharmaceutical composition comprising an isolated immune cell, e.g., a T-cell, that has been pretreated with a sialidase. In another aspect, the invention provides a method of treating cancer in a subject in need thereof, the method comprising administering to the subject an isolated immune cell, e.g., a T-cell, modified to have sialidase activity or modified to have up regulated or increased functional activity associated with or modulated by a sialidase. In another aspect, the invention provides a method of treating cancer in a subject in need thereof, the method comprising administering to the subject an isolated immune cell, e.g., a T-cell, and a sialidase. In another aspect, the invention provides a method of treating cancer in a subject in need thereof, the method comprising administering to the subject an isolated immune cell, e.g., a T-cell, pretreated with a sialidase.

Various features and aspects of the invention are discussed in more detail below.

### I. Immune Cells

Among other things, the invention provides methods and compositions comprising an isolated immune cell useful in the treatment of cancer where the immune cell can be (i) used as is in combination with a sialidase or (ii) engineered to have sialidase activity or up regulated or otherwise increased sialidase activity relative to a similar or identical cell that has not been modified. Immune cells include, e.g., lymphocytes, such as B-cells and T-cells, natural killer cells, myeloid cells, such as monocytes, macrophages, eosinophils, mast cells, basophils, and granulocytes.

In certain embodiments, the immune cell is a T-cell, which can be, for example, a cultured T-cell, e.g., a primary T-cell, or a T-cell from a cultured T-cell line, e.g., Jurkat, SupTi, etc., or a T-cell obtained from a mammal, for example, from a subject to be treated. If obtained from a mammal, the T-cell can be obtained from numerous sources, including but not limited to blood, bone marrow, lymph node, the thymus, or other tissues or fluids. T-cells can also be enriched or purified. The T-cell can be any type of T-cell and can be of any developmental stage, including but not limited to, CD4+/CD8+ double positive T-cells, CD4+ helper T-cells, e.g., Th1 and Th2 cells, CD4+ T-cells, CD8+ T-cells (e.g., cytotoxic T-cells), tumor infiltrating lymphocytes (TILs), memory T-cells (e.g., central memory T-cells and effector memory T-cells), naive T-cells, and the like. The cells (e.g., the T-cells) can include autologous cells derived from a subject to be treated, or alternatively allogenic cells derived from a donor.

In certain embodiments, the T-cell binds an antigen, e.g., a cancer antigen, through a T-cell receptor. The T-cell receptor may be an endogenous or a recombinant T-cell receptor. T-cell receptors comprise two chains referred to as the α- and β-chains, that combine on the surface of a T-cell to form a heterodimeric receptor that can recognize MHC-restricted antigens. Each of α- and β-chain comprises two regions, a constant region and a variable region. Each variable region of the α- and β-chains defines three loops, referred to as complementary determining regions (CDRs) known as CDR₁, CDR₂, and CDR₃ that confer the T-cell receptor with antigen binding activity and binding specificity.

In certain embodiments, the immune cell, e.g., T-cell or NK-cell, binds to an antigen, e.g., a cancer antigen, through a chimeric antigen receptor (CAR), i.e., the T-cell or NK-cell comprises an exogenous nucleotide sequence encoding a CAR. As used herein, the terms “chimeric antigen receptor,” or “CAR,” refer to any artificial receptor including an antigen-specific binding moiety and one or more signaling chains derived from an immune receptor. CARs can comprise a single chain fragment variable (scFv) of an antibody specific for an antigen coupled via hinge and transmembrane regions to cytoplasmic domains of T-cell signaling molecules (e.g. a T-cell costimulatory domain (e.g., from CD28, CD137, OX40, ICOS, or CD27) in tandem with a T-cell triggering domain (e.g. from CD3ζ)) and/or to cytoplasmic domains of NK-cell signaling molecules (e.g. DNAX-activation protein 12 (DAP12)). A T-cell expressing a chimeric antigen receptor is referred to as a CAR T-cell and an NK-cell expressing a chimeric antigen receptor is referred to as a CAR NK-cell.

Exemplary CAR T-cells include CD19 targeted CTL019 cells (see, Grupp et al. (2015) BLOOD 126:4983), 19-28z cells (see, Park et al. (2015) J. CLIN. ONCOL. 33(15S):7010), and KTE-C19 cells (see, Locke et al. (2015) BLOOD 126:3991). Exemplary mesothelin targeted CAR T-cells are described in International (PCT) Publication Nos. WO2013142034, WO2015188141, and WO2017040945. Additional exemplary CAR T-cells are described in U.S. Pat. Nos. 8,399,645, 8,906,682, 7,446,190, 9,181,527, 9,272,002, and 9,266,960, U.S. Patent Publication Nos. US20160362472, US20160200824, and US20160311917 and International (PCT) Publication No. WO2015120180. Engineered immune cells containing a T-cell receptor knockout and a chimeric antigen receptor that binds CD123 are described in International (PCT) Publication No. WO2016120220.

In certain embodiments, a CAR binds a cancer antigen selected from mesothelin, prostate specific membrane antigen (PSMA), prostate stem cell antigen (PCSA), carbonic anhydrase IX (CAIX), carcinoembryonic antigen (CEA), CD5, CD7, CD10, CD19, CD20, CD22, CD30, CD33, CD34, CD38, CD41, CD44, CD47, CD49f, CD56, CD74, CD123, CD133, CD138, epithelial glycoprotein2 (EGP 2), epithelial glycoprotein-40 (EGP-40), epithelial cell adhesion molecule (EpCAM), folate-binding protein (FBP), fetal acetylcholine receptor (AChR), folate receptor-α and β (FRa and β), Ganglioside G2 (GD2), Ganglioside G3 (GD3), an Epidermal Growth Factor Receptor (EGFR), Epidermal Growth Factor Receptor 2 (HER-2/ERB2), Epidermal Growth Factor Receptor vIII (EGFRvIII), ERB3, ERB4, human telomerase reverse transcriptase (hTERT), Interleukin-13 receptor subunit alpha-2 (IL-13Ra2), K-light chain, kinase insert domain receptor (KDR), Lewis A (CA19.9), Lewis Y (LeY), LI cell adhesion molecule (LICAM), melanoma-associated antigen 1 (melanoma antigen family A1, MAGE-A1), Mucin 16 (MUC-16), Mucin 1 (MUC-1), KG2D ligands, cancer-testis antigen NY-ESO-1, oncofetal antigen (h5T4), tumor-associated glycoprotein 72 (TAG-72), vascular endothelial growth factor R2 (VEGF-R2), Wilms tumor protein (WT-1), type 1 tyrosine-protein kinase transmembrane receptor (ROR1), B7-H3 (CD276), B7-H6 (Nkp30), Chondroitin sulfate proteoglycan-4 (CSPG4), DNAX Accessory Molecule (DNAM-1), Ephrin type A Receptor 2 (EpHA2), Fibroblast Associated Protein (FAP), Gp100/HLA-A2, Glypican 3 (GPC3), HA-IH, HERK-V, IL-1 IRa, Latent Membrane Protein 1 (LMP1), Neural cell-adhesion molecule (N-CAM/CD56), programmed cell death receptor ligand 1 (PD-L1), B Cell Maturation Antigen (BCMA), and Trail Receptor (TRAIL R). In certain embodiments, a CAR binds a cancer antigen selected from mesothelin, HER-2/ERB2, EGFR, CD20, PD-L1, CEA, EpCAM, GPC3, BCMA, CD47, CD38 and MUC-1.

### II. Sialidases

In certain embodiments, an immune cell, e.g., a T-cell, is modified to express a sialidase, or is administered in combination with a sialidase.

As used herein, the term “sialidase” refers to any enzyme, or a functional fragment thereof, that cleaves a terminal sialic acid residue from a substrate, for example, a glycoprotein or a glycolipid. As used herein, a “functional fragment” of a sialidase refers to fragment of a full-length sialidase that retains, for example, at least 10%, at least 20%, at least 30%, at least 40%, at least 50%, at least 60%, at least 70%, at least 80%, at least 90%, or 100% of the enzymatic activity of the corresponding full-length sialidase. Sialidase enzymatic activity may be assayed by any method known in the art, including, for example, by measuring the release of sialic acid from the fluorogenic substrate 4-methylumbelliferyl-N-acetylneuraminic acid (4MU-NeuAc). In certain embodiments, the functional fragment comprises at least 100, 150, 200, 250, 300, 310, 320, 330, 340, 350, 360, or 370 consecutive amino acids present in a full-length sialidase. The term sialidase includes variants having one or more amino acid substitutions, deletions, or insertions relative to a wild-type sialidase sequence, and/or fusion proteins or conjugates including a sialidase. Sialidases are also called neuraminidases, and, unless indicated otherwise, the two terms are used interchangeably herein.

In certain embodiments, the sialidase is a eukaryotic sialidase, e.g., a mammalian sialidase, e.g., a human sialidase. Four sialidases have been found in the human genome and are referred to as Neu1, Neu2, Neu3 and Neu4.

Neu1 is a lysosomal neuraminidase enzyme which functions in a complex with beta-galactosidase and cathepsin A. The amino acid sequence of human Neu1 is depicted in SEQ ID NO: 1, and a nucleotide sequence encoding human Neu1 is depicted in SEQ ID NO: 9.

Neu2 is a cytosolic sialidase enzyme. The amino acid sequence of human Neu2 is depicted in SEQ ID NO: 2, and a nucleotide sequence encoding human Neu2 is depicted in SEQ ID NO: 10.

Neu3 is a plasma membrane sialidase with an activity specific for gangliosides. Neu3 has two isoforms: isoform 1 and isoform 2. The amino acid sequence of human Neu3, isoform 1 is depicted in SEQ ID NO: 3, and a nucleotide sequence encoding human Neu3, isoform 1 is depicted in SEQ ID NO: 11. The amino acid sequence of human Neu3, isoform 4 is depicted in SEQ ID NO: 4, and a nucleotide sequence encoding human Neu3, isoform 2 is depicted in SEQ ID NO: 12.

Neu4 has two isoforms: isoform 1 is a peripheral membrane protein and isoform 2 localizes to the lysosome lumen. The amino acid sequence of human Neu4, isoform 1 is depicted in SEQ ID NO: 5, and a nucleotide sequence encoding human Neu4, isoform 1 is depicted in SEQ ID NO: 13. The amino acid sequence of human Neu4, isoform 2 is depicted in SEQ ID NO: 6, and a nucleotide sequence encoding human Neu4, isoform 2 is depicted in SEQ ID NO: 14.

Four sialidases have also been found in the mouse genome and are referred to as Neu1, Neu2, Neu3 and Neu4. The amino acid sequence of mouse Neu1 is depicted in SEQ ID NO: 34, and a nucleotide sequence encoding mouse Neu1 is depicted in SEQ ID NO: 58. The amino acid sequence of mouse Neu2 is depicted in SEQ ID NO: 35 and a nucleotide sequence encoding mouse Neu2 is depicted in SEQ ID NO: 59. The amino acid sequence of mouse Neu3 is depicted in SEQ ID NO: 56, and a nucleotide sequence encoding mouse Neu3 is depicted in SEQ ID NO: 60. The amino acid sequence of mouse Neu4 is depicted in SEQ ID NO: 57, and a nucleotide sequence encoding mouse Neu4 is depicted in SEQ ID NO: 61.

In certain embodiments, the sialidase is a prokaryotic sialidase. Exemplary prokaryotic sialidases include sialidases from Salmonella typhimurium and Vibrio cholera. The amino acid sequence of Salmonella typhimurium sialidase (St-sialidase) is depicted in SEQ ID NO: 7, and a nucleotide sequence encoding Salmonella typhimurium sialidase is depicted in SEQ ID NO: 15. The amino acid sequence of Vibrio cholera sialidase is depicted in SEQ ID NO: 8, and a nucleotide sequence encoding Vibrio cholera sialidase is depicted in SEQ ID NO: 16.

In certain embodiments the sialidase can cleave α2,3, α2,6, and/or α2,8 linkages. In certain embodiments the sialidase can cleave α2,3 and α2,8 linkages.

In certain embodiments, the sialidase comprises the amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, or SEQ ID NO: 8, or an amino acid sequence that has at least 90%, 95%, 96%, 97%, 98%, or 99% sequence identity to SEQ ID NO: 1, SEQ ID NO: 2, SEQ ID NO: 3, SEQ ID NO: 4, SEQ ID NO: 5, SEQ ID NO: 6, SEQ ID NO: 7, or SEQ ID NO: 8. Sequence identity may be determined in various ways that are within the skill of a person skilled in the art, e.g., using publicly available computer software such as BLAST, BLAST-2, ALIGN or Megalign (DNASTAR) software. BLAST (Basic Local Alignment Search Tool) analysis using the algorithm employed by the programs blastp, blastn, blastx, tblastn and tblastx (Karlin et al., (1990) PROC. NATL. ACAD. SCI. USA 87:2264-2268; Altschul, (1993) J. MOL. EVOL. 36:290-300; Altschul et al., (1997) NUCLEIC ACIDS RES. 25:3389-3402, incorporated by reference herein) are tailored for sequence similarity searching. For a discussion of basic issues in searching sequence databases see Altschul et al., (1994) NATURE GENETICS 6:119-129, which is fully incorporated by reference herein. Those skilled in the art can determine appropriate parameters for measuring alignment, including any algorithms needed to achieve maximal alignment over the full length of the sequences being compared. The search parameters for histogram, descriptions, alignments, expect (i.e., the statistical significance threshold for reporting matches against database sequences), cutoff, matrix and filter are at the default settings. The default scoring matrix used by blastp, blastx, tblastn, and tblastx is the BLOSUM62 matrix (Henikoff et al., (1992) PROC. NATL. ACAD. Sci. USA 89:10915-10919, fully incorporated by reference herein). Four blastn parameters may be adjusted as follows: Q=10 (gap creation penalty); R=10 (gap extension penalty); wink=1 (generates word hits at every wink.sup.th position along the query); and gapw=16 (sets the window width within which gapped alignments are generated). The equivalent blastp parameter settings may be Q=9; R=2; wink=1; and gapw=32. Searches may also be conducted using the NCBI (National Center for Biotechnology Information) BLAST Advanced Option parameter (e.g.: -G, Cost to open gap [Integer]: default=5 for nucleotides/11 for proteins; −E, Cost to extend gap [Integer]: default=2 for nucleotides/1 for proteins; −q, Penalty for nucleotide mismatch [Integer]: default=−3; −r, reward for nucleotide match [Integer]: default=1; −e, expect value [Real]: default=10; −W, wordsize [Integer]: default=11 for nucleotides/28 for megablast/3 for proteins; −y, Dropoff (X) for blast extensions in bits: default=20 for blastn/7 for others; —X, X dropoff value for gapped alignment (in bits): default=15 for all programs, not applicable to blastn; and —Z, final X dropoff value for gapped alignment (in bits): 50 for blastn, 25 for others). ClustalW for pairwise protein alignments may also be used (default parameters may include, e.g., Blosum62 matrix and Gap Opening Penalty=10 and Gap Extension Penalty=0.1). A Bestfit comparison between sequences, available in the GCG package version 10.0, uses DNA parameters GAP=50 (gap creation penalty) and LEN=3 (gap extension penalty). The equivalent settings in Bestfit protein comparisons are GAP=8 and LEN=2.

### Mutant Sialidases

In certain embodiments, the sialidase is a mutant sialidase. In certain embodiments, the mutant sialidase comprises a substitution of at least one wild-type amino acid residue, wherein the substitution increases at least one of the (a) expression, (b) stability and (c) activity of the sialidase, or a combination of (a) and (b), combination of (a) and (c), a combination of (b) and (c), or a combination of (a), (b) and (c). In certain embodiments, a mutant sialidase has about 5%, about 10%, about 15%, about 20%, about 25%, about 30%, about 35%, about 40%, about 45%, about 50%, about 60%, about 65%, about 70%, about 75%, about 80%, about 85%, about 90%, about 95%, about 100%, or more than 100% of the enzymatic activity of a corresponding (or template) wild-type sialidase. In certain embodiments, a mutant sialidase has a different substrate specificity than the corresponding wild-type sialidase. In certain embodiments, the expression yield of the mutant sialidase in mammalian cells, e.g., HEK293 cells, CHO cells, murine myeloma cells (NS0, Sp2/0), or human fibrosarcoma cells (HT-1080), e.g., HEK293 cells, is greater than about 10%, about 20%, about 50%, about 75%, about 100%, about 150%, about 200%, about 250%, about 300%, about 400%, about 500%, about 600%, about 700%, about 800%, about 900%, or about 1,000% of the expression yield of the corresponding wild-type sialidase. In certain embodiments, a mutant sialidase has at least 50%, 55%, 60%, 65%, 70%, 75%, 80%, 85%, 90%, 95%, 96%, 97%, 98%, or 99% sequence identity to the amino acid sequence of a corresponding wild-type sialidase.

In certain embodiments, the mutant sialidase comprises a substitution of at least one cysteine (cys, C) residue. In certain embodiments, the mutant sialidase contains at least one mutation of a free cysteine (e.g., for human Neu1 (SEQ ID NO: 1), C111, C117, C171, C183, C218, C240, C242, and C252; for human Neu2 (SEQ ID NO: 2), C125, C196, C219, C272, C332, and C352; for Neu3 (SEQ ID NO: 3), C7, C90, C99, C106, C127, C136, C189, C194, C226, C242, C250, C273, C279, C295, C356, C365, C368, C384, C383, C394, and C415; and for Neu4 (SEQ ID NO: 5), C88, C125, C126, C186, C191, C211, C223, C239, C276, C437, C453, C480, and C481). Free cysteines can be substituted with any amino acid. In certain embodiments, the free cysteine is substituted with serine (ser, S), isoleucine (iso, I), valine (val, V), phenylalanine (phe, F), leucine (leu, L), or alanine (ala, A). Exemplary cysteine substitutions in human Neu2 include C125A, C125I, C125S, C125V, C196A, C196L, C196V, C272S, C272V, C332A, C332S, C332V, C352L, and C352V. In certain embodiments, the mutant sialidase comprises two or more cysteine substitutions. Exemplary double or triple substitutions in human Neu2 include: C125S and C332S; C272V and C332A; C272V and C332S; C332A and C352L; C125S and C196L; C196L and C352L; C196L and C332A; C332A and C352L; and C196L, C332A and C352L. In certain embodiments, the mutant sialidase is a human Neu2 sialidase and comprises the substitutions C322A and C352L. In certain embodiments, the mutant sialidase contains an amino acid substitution at 2, 3, 4, 5, or 6 cysteines typically present in a human sialidase, e.g., Neu2 or Neu3.

In certain embodiments, the mutant sialidase comprises at least one amino acid substitution, wherein the substitution increases the isoelectric point (pI) of the sialidase and/or decreases the hydrophobicity of the sialidase relative to a sialidase without the substitution. This may be achieved by introducing one or more charged amino acids, for example, positively or negatively charged amino acids, into the sialidase. In certain embodiments, the amino acid substitution is to a charged amino acid, for example, a positively charged amino acid such as lysine (lys, K), histidine (his, H), or arginine (arg, R), or a negatively charged amino acid such as aspartic acid (asp, D) or glutamic acid (glu, E). In certain embodiments, the amino acid substitution is to a lysine residue. In certain embodiments, the substitution increases the pI of the sialidase to about 7.75, about 8, about 8.25, about 8.5, about 8.75, about 9, about 9.25, about 9.5, or about 9.75. In certain embodiments, the amino acid substitution occurs at a surface exposed D or E amino acid, in a helix or loop, or in a position that has a K or R in the corresponding position of St-sialidase. In certain embodiments, the amino acid substitution occurs at an amino acid that is remote from the catalytic site or otherwise not involved in catalysis, an amino acid that is not conserved with the other human Neu proteins or with an St-Sialidase or Clostridium NanH, or an amino acid that is not located in a domain important for function (e.g., an Asp-box or beta strand). Exemplary amino acid substitutions in human Neu2 that increase the isoelectric point (pI) of the sialidase and/or decrease the hydrophobicity of the sialidase relative to a sialidase without the substitution include A2E, A2K, D215K, V325E, V325K, E257K, and E319K. In certain embodiments, the mutant sialidase comprises two or more amino acid substitutions, including, for example, A2K and V325E, A2K and V325K, E257K and V325K, A2K and E257K, and E257K and A2K and V325K.

In certain embodiments, the N-terminal six amino acids of the mouse thymus Neu2 isoform, MEDLRP (SEQ ID NO: 17), or variations thereof, can be added onto a human Neu, e.g., human Neu2. In certain embodiments, the mutant sialidase comprises a peptide at least two amino acid residues in length covalently associated with an N-terminal amino acid of the sialidase. In certain embodiments the mutant sialidase comprises the peptide MEDLRP (SEQ ID NO: 17) or EDLRP (SEQ ID NO: 18) covalently associated with an N-terminal amino acid of the sialidase. In certain embodiments, the sialidase may further comprise a cleavage site, e.g., a proteolytic cleavage site, located between the peptide, e.g., MEDLRP (SEQ ID NO: 17) or EDLRP (SEQ ID NO: 18), and the remainder of the sialidase. In certain embodiments, the peptide, e.g., MEDLRP (SEQ ID NO: 17) or EDLRP (SEQ ID NO: 18), may be postranslationally cleaved from the remainder of the sialidase.

Alternatively to, or in combination with, the N-terminal addition, 1-5 amino acids of the 12 amino acid N-terminal region of the mutant sialidase may be removed, e.g., the N-terminal methionine can be removed. In certain embodiments, if the sialidase is human Neu2, the N-terminal methionine can be removed, the first five amino acids (MASLP; SEQ ID NO: 19) can be removed, or the second through fourth amino acids (ASLP; SEQ ID NO: 20) can be removed. In certain embodiments, 1-5 amino acids of the 12 amino acid N-terminal region of the mutant sialidase are substituted with MEDLRP (SEQ ID NO: 17), EDLRP (SEQ ID NO: 18), or TVEKSVVF (SEQ ID NO: 21). For example, in certain embodiments, if the sialidase is human Neu2, the amino acids MASLP (SEQ ID NO: 19), ASLP (SEQ ID NO: 20) or M are substituted with MEDLRP (SEQ ID NO: 17), EDLRP (SEQ ID NO: 18) or TVEKSVVF (SEQ ID NO: 21). In certain embodiments, the sialidase comprises a LSHSLST (SEQ ID NO: 79) peptide on the N-terminus.

In certain embodiments, the mutant sialidase comprises a substitution of at least one wild-type amino acid residue, wherein the substitution increases hydrophobic interactions and/or hydrogen bonding between the N- and C-termini of the sialidase relative to a sialidase without the substitution. In certain embodiments, the wild-type amino acid is substituted with asparagine (asn, N), lysine (lys, K), tyrosine (tyr, Y), phenylalanine (phe, F), or tryptophan (trp, W). Exemplary substitutions in human Neu2 that increase hydrophobic interactions and/or hydrogen bonding between the N- and C-termini include L4N, L4K, V6Y, L7N, L4N and L7N, L4N and V6Y and L7N, V12N, V12Y, V12L, V6Y, V6F, or V6W. In certain embodiments, the sialidase comprises the V6Y substitution.

In certain embodiments, the mutant sialidase comprises a combination of the above substitutions. For example, a mutant human Neu2 sialidase can comprise the additional amino acids MEDLRP (SEQ ID NO: 17), EDLRP (SEQ ID NO: 18), or TVEKSVVF (SEQ ID NO: 21) at the N-terminus and, in combination, can comprise at least one L4N, L4K, V6Y, L7N, L4N and L7N, L4N and V6Y and L7N, V12N, V12Y, V12L, V6Y, V6F, or V6W substitution. In certain embodiments, the amino acids MASLP (SEQ ID NO: 19), ASLP (SEQ ID NO: 20) or M of a mutant human Neu2 sialidase are replaced with MEDLRP (SEQ ID NO: 17), EDLRP (SEQ ID NO: 18) or TVEKSVVF (SEQ ID NO: 21) and the mutant human Neu2 sialidase also comprises at least one L4N, L4K, V6Y, L7N, L4N and L7N, L4N and V6Y and L7N, V12N, V12Y, V12L, V6Y, V6F, or V6W substitution.

Additionally, in certain embodiments, the sialidase comprises a substitution or deletion of an N-terminal methionine at the N-terminus of the sialidase. For example, in certain embodiments, the sialidase comprises a substitution of a methionine residue at a position corresponding to position 1 of wild-type human Neu2 (SEQ ID NO: 2), e.g., the methionine at a position corresponding to position 1 of wild-type human Neu2 is substituted by alanine (M1A) or aspartic acid (M1D). In other embodiments, the sialidase comprises a deletion of a methionine residue at a position corresponding to position 1 (ΔM1) of wild-type human Neu2 (SEQ ID NO: 2).

In certain embodiments, a mutant human Neu2 sialidase comprises at least one of the following substitutions: I187K, A328E, K370N, or H210N. In certain embodiments, a mutant human Neu2 sialidase comprises the substitution of the amino acids GDYDAPTHQVQW (SEQ ID NO: 22) with the amino acids SMDQGSTW (SEQ ID NO: 23) or STDGGKTW (SEQ ID NO: 24). In certain embodiments, a mutant human Neu2 sialidase comprises the substitution of the amino acids PRPPAPEA (SEQ ID NO: 25) with the amino acids QTPLEAAC (SEQ ID NO: 26). In certain embodiments, a mutant human Neu2 sialidase comprises the substitution of the amino acids NPRPPAPEA (SEQ ID NO: 27) with the amino acids SQNDGES (SEQ ID NO: 28). In certain embodiments, a mutant human Neu2 sialidase comprises at least one substitution at a position corresponding to V212, A213, Q214, D215, T216, L217, E218, C219, Q220, V221, A222, E223, V224, E225, or T225.

In certain embodiments, a mutant sialidase comprises: (a) a substitution of a proline residue at a position corresponding to position 5 of wild-type human Neu2 (P5); (b) a substitution of a lysine residue at a position corresponding to position 9 of wild-type human Neu2 (K9); (c) a substitution of a lysine residue at a position corresponding to position 44 of wild-type human Neu2 (K44); (d) a substitution of a lysine residue at a position corresponding to position 45 of wild-type human Neu2 (K45); (e) a substitution of a leucine residue at a position corresponding to position 54 of wild-type human Neu2 (L54); (f) a substitution of a proline residue at a position corresponding to position 62 of wild-type human Neu2 (P62); (g) a substitution of a glutamine residue at a position corresponding to position 69 of wild-type human Neu2 (Q69); (h) a substitution of an arginine residue at a position corresponding to position 78 of wild-type human Neu2 (R78); (i) a substitution of an alanine residue at a position corresponding to position 93 of wild-type human Neu2 (A93); (j) a substitution of a glycine residue at a position corresponding to position 107 of wild-type human Neu2 (G107); (k) a substitution of a glutamine residue at a position corresponding to position 108 of wild-type human Neu2 (Q108); (l) a substitution of a glutamine residue at a position corresponding to position 112 of wild-type human Neu2 (Q112); (m) a substitution of a cysteine residue at a position corresponding to position 125 of wild-type human Neu2 (C125); (n) a substitution of a glutamine residue at a position corresponding to position 126 of wild-type human Neu2 (Q126); (o) a substitution of an alanine residue at a position corresponding to position 150 of wild-type human Neu2 (A150); (p) a substitution of a cysteine residue at a position corresponding to position 164 of wild-type human Neu2 (C164); (q) a substitution of an alanine residue at a position corresponding to position 171 of wild-type human Neu2 (A171); (r) a substitution of a leucine residue at a position corresponding to position 217 of wild-type human Neu2 (L217); (s) a substitution of a threonine residue at a position corresponding to position 249 of wild-type human Neu2 (T249); (t) a substitution of an aspartic acid residue at a position corresponding to position 251 of wild-type human Neu2 (D251); (u) a substitution of a glutamine residue at a position corresponding to position 270 of wild-type human Neu2 (Q270); (v) a substitution of a tryptophan residue at a position corresponding to position 292 of wild-type human Neu2 (W292); (w) a substitution of a serine residue at a position corresponding to position 301 of wild-type human Neu2 (S301); (x) a substitution of a tryptophan residue at a position corresponding to position 302 of wild-type human Neu2 (W302); (y) a substitution of a valine residue at a position corresponding to position 363 of wild-type human Neu2 (V363); or (z) a substitution of a leucine residue at a position corresponding to position 365 of wild-type human Neu2 (L365); or a combination of any of the foregoing substitutions. For example, the sialidase may comprise a substitution of K9, P62, A93, Q270, S301, W302, V363, or L365, or a combination of any of the foregoing substitutions.

In certain embodiments, in the sialidase: (a) the proline residue at a position corresponding to position 5 of wild-type human Neu2 is substituted by histidine (P5H); (b) the lysine residue at a position corresponding to position 9 of wild-type human Neu2 is substituted by aspartic acid (K9D); (c) the lysine residue at a position corresponding to position 44 of wild-type human Neu2 is substituted by arginine (K44R) or glutamic acid (K44E); (d) the lysine residue at a position corresponding to position 45 of wild-type human Neu2 is substituted by alanine (K45A), arginine (K45R), or glutamic acid (K45E); (e) the leucine residue at a position corresponding to position 54 of wild-type human Neu2 is substituted by methionine (L54M); (f) the proline residue at a position corresponding to position 62 of wild-type human Neu2 is substituted by asparagine (P62N), aspartic acid (P62D), histidine (P62H), glutamic acid (P62E), glycine (P62G), serine (P62S), or threonine (P62T); (g) the glutamine residue at a position corresponding to position 69 of wild-type human Neu2 is substituted by histidine (Q69H); (h) the arginine residue at a position corresponding to position 78 of wild-type human Neu2 is substituted by lysine (R78K); (i) the alanine residue at a position corresponding to position 93 of wild-type human Neu2 is substituted by glutamic acid (A93E) or lysine (A93K); (j) the glycine residue at a position corresponding to position 107 of wild-type human Neu2 is substituted by aspartic acid (G107D); (k) the glutamine residue at a position corresponding to position 108 of wild-type human Neu2 is substituted by histidine (Q108H); (l) the glutamine residue at a position corresponding to position 112 of wild-type human Neu2 is substituted by arginine (Q112R) or lysine (Q112K); (m) the cysteine residue at a position corresponding to position 125 of wild-type human Neu2 is substituted by leucine (C125L); (n) the glutamine residue at a position corresponding to position 126 of wild-type human Neu2 is substituted by leucine (Q126L); (o) the alanine residue at a position corresponding to position 150 of wild-type human Neu2 is substituted by valine (A150V); (p) the cysteine residue at a position corresponding to position 164 of wild-type human Neu2 is substituted by glycine (C164G); (q) the alanine residue at a position corresponding to position 171 of wild-type human Neu2 is substituted by glycine (A171G); (r) the leucine residue at a position corresponding to position 217 of wild-type human Neu2 is substituted by alanine (L217A) or valine (L217V); (s) the threonine residue at a position corresponding to position 249 of wild-type human Neu2 is substituted by alanine (T249A); (t) the aspartic acid residue at a position corresponding to position 251 of wild-type human Neu2 is substituted by glycine (D251G); (u) the glutamine residue at a position corresponding to position 270 of wild-type human Neu2 is substituted by alanine (Q270A), histidine (Q270H), phenylalanine (Q270F) or proline (Q270P); (v) the tryptophan residue at a position corresponding to position 292 of wild-type human Neu2 is substituted by arginine (W292R); (w) the serine residue at a position corresponding to position 301 of wild-type human Neu2 is substituted by arginine (S301R); (x) the tryptophan residue at a position corresponding to position 302 of wild-type human Neu2 is substituted by lysine (W302K); (y) the valine residue at a position corresponding to position 363 of wild-type human Neu2 is substituted by arginine (V363R); or (z) the leucine residue at a position corresponding to position 365 of wild-type human Neu2 is substituted by glutamine (L365Q), histidine (L365H), isoleucine (L365I), lysine (L365K) or serine (L365S), or the sialidase comprises a combination of any of the foregoing substitutions. For example, the sialidase may comprise the K9D, P62G, P62N, P62S, P62T, A93E, Q270A, S301R, W302K, V363R, or L365I substitutions, or a combination of any of the foregoing substitutions.

In certain embodiments, a mutant sialidase comprises a deletion of a leucine residue at a position corresponding to position 184 of wild-type human Neu2 (ΔL184), a deletion of a histidine residue at a position corresponding to position 185 of wild-type human Neu2 (ΔH185), a deletion of a proline residue at a position corresponding to position 186 of wild-type human Neu2 (ΔP186), a deletion of an isoleucine residue at a position corresponding to position 187 of wild-type human Neu2 (ΔI187), or a deletion of a glutamine residue at a position corresponding to position 184 of wild-type human Neu2 (ΔQ188), or a combination of any of the foregoing deletions.

In certain embodiments, a mutant sialidase comprises an insertion between a threonine residue at a position corresponding to position 216 of wild-type human Neu2 and a leucine residue at a position corresponding to position 217 of wild-type human Neu2, for example, an insertion of an amino acid selected from S, T, Y, L, F, A, P, V, I, N, D, and H.

Additional exemplary sialidase mutations, and combinations of sialidase mutations, are described in International (PCT) Patent Application No. PCT/US2019/012207, filed Jan. 3, 2019, including in the Detailed Description in the section entitled “I. Recombinant Human Sialidases,” and in the Examples in Examples 1, 2, 3, 4, 5, and 6. Additional exemplary sialidase mutations, and combinations of sialidase mutations, are described in U.S. Provisional Patent Application No. 62/870,403, filed Jul. 3, 2019, including in the Detailed Description in the section entitled “I. Recombinant Human Sialidases,” and in the Examples in Examples 2, 3, 4, and 8.

In certain embodiments, a mutant sialidase comprises a combination of any of the mutations contemplated herein. For example, the mutant sialidase enzyme may comprise a combination of 2, 3, 4, 5, 6, 7, 8, 9, 10, or more of the mutations contemplated herein. For example, the mutant sialidase may comprise a M1 deletion (ΔM1), M1A substitution, M1D substitution, V6Y substitution, K9D substitution, P62G substitution, P62N substitution, P62S substitution, P62T substitution, A93E substitution, I187K substitution, Q270A substitution, S301R substitution, W302K substitution, C332A substitution, V363R substitution, L365I substitution, or a combination of any of the foregoing. For example, the mutant sialidase enzyme may comprise a M1 deletion (ΔM1), M1A substitution, M1D substitution, V6Y substitution, I187K substitution, C332A substitution, or a combination of any of the foregoing. For example, the mutant sialidase enzyme may comprise a combination of mutations selected from: M1A and V6Y; M1A and I187K; M1A and C332A; M1D and V6Y; M1D and I187K; M1D and C332A; ΔM1 and V6Y; ΔM1 and I187K; ΔM1 and C332A; V6Y and I187K; V6Y and C332A; I187K and C332A; M1A, V6Y, and I187K; M1A, V6Y, and C332A; M1A, I187K, and C332A; M1D, V6Y, and I187K; M1D, V6Y, and C332A; M1D, I187K, and C332A; ΔM1, V6Y, and I187K; ΔM1, V6Y, and C332A; ΔM1, I187K, and C332A; V6Y, I187K, and C332A; M1A, V6Y, I187K, and C332A; M1D, V6Y, I187K, and C332A; and ΔM1, V6Y, I187K, and C332A. In certain embodiments, the mutant sialidase comprises: (a) the M1D, V6Y, P62G, A93E, I187K, and C332A substitutions; (b) the M1D, V6Y, K9D, A93E, I187K, C332A, V363R, and L365I substitutions; (c) the M1D, V6Y, P62N, I187K, and C332A substitutions; (d) the M1D, V6Y, I187K, Q270A, S301R, W302K, and C332A substitutions; (e) the M1D, V6Y, P62S, I187K, Q270A, S301R, W302K, and C332A substitutions; (f) the M1D, V6Y, P62T, I187K, Q270A, S301R, W302K, and C332A substitutions; or (g) the M1D, V6Y, P62N, I187K, Q270A, S301R, W302K, and C332A substitutions.

In certain embodiments, the sialidase comprises the amino acid sequence of SEQ ID NO: 36, SEQ ID NO: 37, SEQ ID NO: 38, SEQ ID NO: 39, SEQ ID NO: 63, SEQ ID NO: 64, SEQ ID NO: 65, SEQ ID NO: 66, SEQ ID NO: 83, SEQ ID NO: 84, SEQ ID NO: 85, SEQ ID NO: 86, SEQ ID NO: 87, SEQ ID NO: 88, or SEQ ID NO: 89, or an amino acid sequence that has at least 85%, 90%, 95%, 96%, 97%, 98%, or 99% sequence identity to SEQ ID NO: 36, SEQ ID NO: 37, SEQ ID NO: 38, SEQ ID NO: 39, SEQ ID NO: 63, SEQ ID NO: 64, SEQ ID NO: 65, SEQ ID NO: 66, SEQ ID NO: 83, SEQ ID NO: 84, SEQ ID NO: 85, SEQ ID NO: 86, SEQ ID NO: 87, SEQ ID NO: 88, or SEQ ID NO: 89.

In certain embodiments, the sialidase comprises the amino acid sequence of

X₁X₂SX₃PX₄LQKESVFQSGAHAYRIPALLYLPGQQSLLAFAEQRASKKDE
HAELIVLRRGDYDAPTHQVQWQAQEVVAQARLDGHRSMNPCPLYDAQTGT
LFLFFIAIPGQVTEQQQLQTRANVTRLX₅QVTSTDHGRTWSSPRDLTDAA
IGPAYREWSTFAVGPGHCLQLHDRARSLVVPAYAYRKLHPX₆QRPIPSAF
X₇FLSHDHGRTWARGHFVAQDTLECQVAEVETGEQRVVTLNARSHLRARV
QAQSTNDGLDFQX₈SQLVKKLVEPPPQGX₉QGSVISFPSPRSGPGSPAQW
LLYTHPTHSWQRADLGAYLNPRPPAPEAWSEPX₁₀LLAKGSX₁₁AYSDLQS
MGTGPDGSPLFGX₁₂LYEANDYEEIVFLMFTLKQAFPAEYLPQ

(SEQ ID NO: 76), wherein X₁ is Ala, Arg, Asn, Asp, Gln, Glu, Gly, His, Leu, Lys, Met, Phe, Thr, Val, or not present, X₂ is Ala or Lys, X₃ is Asn or Leu, X₄ is Phe, Trp, Tyr or Val, X₅ is Ala, Cys, Ile, Ser, or Val, X₆ is Arg, Ile, or Lys, X₇ is Ala, Cys, Leu, or Val, X₈ is Glu or Lys, X₉ is Cys or Val, X₁₀ is Lys or Val, X₁₁ is Ala, Cys, Ser, or Val, and X₁₂ is Cys, Leu, or Val, and the sialidase comprises at least one mutation relative to wild-type human Neu2 (SEQ ID NO: 2).

In certain embodiments, the sialidase comprises the amino acid sequence of

X₁ASLPX₂LQKESVFQSGAHAYRIPALLYLPGQQSLLAFAEQRASKKDEH
AELIVLRRGDYDAPTHQVQWQAQEVVAQARLDGHRSMNPCPLYDAQTGT
LFLFFIAIPGQVTEQQQLQTRANVTRLCQVTSTDHGRTWSSPRDLTDAA
IGPAYREWSTFAVGPGHCLQLHDRARSLVVPAYAYRKLHPX₃QRPIPSA
FCFLSHDHGRTWARGHFVAQDTLECQVAEVETGEQRVVTLNARSHLRAR
VQAQSTNDGLDFQESQLVKKLVEPPPQGCQGSVISFPSPRSGPGSPAQW
LLYTHPTHSWQRADLGAYLNPRPPAPEAWSEPVLLAKGSX₄AYSDLQSM
GTGPDGSPLFGCLYEANDYEEIVFLMFTLKQAFPAEYLPQ

(SEQ ID NO: 73), wherein X₁ is Ala, Asn, Asp, His, Leu, Met, Phe, Thr, or not present, X₂ is Phe, Trp, Tyr or Val, X₃ is Arg, Ile, or Lys, and X₄ is Ala, Cys, Ser, or Val, and the sialidase comprises at least one mutation relative to wild-type human Neu2 (SEQ ID NO: 2). In certain embodiments, X₁ is Ala, Asp, Met, or not present, X₂ is Tyr or Val, X₃ is Ile or Lys, and X₄ is Ala or Cys.

In certain embodiments, the recombinant mutant human sialidase comprises the amino acid sequence of

X₁X₂SX₃X₄X₅LQX₆ESVFQSGAHAYRIPALLYLPGQQSLLAFAEQRASX₇
X₈DEHAELIVX₉RRGDYDAX₁₀THQVQWX₁₁AQEVVAQAX₁₂LDGHRSMNP
CPLYDX₁₃QTGTLFLFFIAIPX₁₄X₁₅VTEX₁₆QQLQTRANVTRLX₁₇X₁₈V
TSTDHGRTWSSPRDLTDAAIGPX₁₉YREWSTFAVGPGHX₂₀LQLHDRX₂₁
RSLVVPAYAYRKLHPX₂₂QRPIPSAFX₂₃FLSHDHGRTWARGHFVAQDT
X₂₄ECQVAEVETGEQRVVTLNARSHLRARVQAQSX₂₅NX₂₆GLDFQX₂₇S
QLVKKLVEPPPX₂₈GX₂₉QGSVISFPSPRSGPGSPAQX₃₀LLYTHPTHX₃₁
X₃₂QRADLGAYLNPRPPAPEAWSEPX₃₃LLAKGSX₃₄AYSDLQSMGTGPD
GSPLFGX₃₅LYEANDYEEIX₃₆FX₃₇MFTLKQAFPAEYLPQ

(SEQ ID NO: 81), wherein X₁ is Ala, Arg, Asn, Asp, Gln, Glu, Gly, His, Leu, Lys, Met, Phe, Thr, Val, or not present, X₂ is Ala or Lys, X₃ is Asn or Leu, X₄ is Pro or His, X₅ is Phe, Trp, Tyr or Val, X₆ is Lys or Asp. X₇ is Lys, Arg, or Glu. X₈ is Lys, Ala, Arg, or Glu, X₉ is Leu or Met, X₁₀ is Pro, Asn, Asp, His, Glu, Gly, Ser or Thr, X₁₁ is Gln or His, X₁₂ is Arg or Lys, X₁₃ is Ala, Glu or Lys, X₁₄ is Gly or Asp, X₁₅ is Gln or His, X₁₆ is Gln, Arg, or Lys, X₁₇ is Ala, Cys, Ile, Ser, Val, or Leu, X₁₈ is Gln or Leu, X₁₉ is Ala or Val, X₂₀ is Cys or Gly, X₂₁ is Ala or Gly, X₂₂ is Arg, Ile, or Lys, X₂₃ is Ala, Cys, Leu, or Val, X₂₄ is Leu, Ala, or Val, X₂₅ is Thr or Ala, X₂₆ is Asp or Gly, X₂₇ is Glu or Lys, X₂₈ is Gln, Ala, His, Phe, or Pro, X₂₉ is Cys or Val, X₃₀ is Trp or Arg, X₃₁ is Ser or Arg, X₃₂ is Trp or Lys, X₃₃ is Lys or Val, X₃₄ is Ala, Cys, Ser, or Val, X₃₅ is Cys, Leu, or Val, X₃₅ is Val or Arg, and X₃₇ is Leu, Gln, His, Ile, Lys, or Ser, and the sialidase comprises at least one mutation relative to wild-type human Neu2 (SEQ ID NO: 2).

In certain embodiments, the recombinant mutant human sialidase comprises the amino acid sequence of

X₁ASLPX₂LQX₃ESVFQSGAHAYRIPALLYLPGQQSLLAFAEQRASKKDEH
AELIVLRRGDYDAX₄THQVQWQAQEVVAQARLDGHRSMNPCPLYDX₅QTG
TLFLFFIAIPGQVTEQQQLQTRANVTRLCQVTSTDHGRTWSSPRDLTDA
AIGPAYREWSTFAVGPGHCLQLHDRARSLVVPAYAYRKLHPX₆QRPIPS
AFCFLSHDHGRTWARGHFVAQDTLECQVAEVETGEQRVVTLNARSHLRA
RVQAQSTNDGLDFQESQLVKKLVEPPPX₇GCQGSVISFPSPRSGPGSPAQ
WLLYTHPTHX₈X₉QRADLGAYLNPRPPAPEAWSEPVLLAKGSX₁₀AYSDLQ
SMGTGPDGSPLFGCLYEANDYEEIX₁₁FX₁₂MFTLKQAFPAEYLPQ

(SEQ ID NO: 82), wherein X₁ is Ala, Arg, Asn, Asp, Gln, Glu, Gly, His, Leu, Lys, Met, Phe, Thr, Val, or not present, X₂ is Phe, Trp, Tyr or Val, X₃ is Lys or Asp, X₄ is Pro, Asn, Asp, His, Glu, Gly, Ser or Thr, X₅ is Ala, Glu, or Lys, X₆ is Arg, Ile, or Lys, X₇ is Gln, Ala, His, Phe, or Pro, X₈ is Ser or Arg, X₉ is Trp or Lys, X₁₀ is Ala, Cys, Ser, or Val, X₁₁ is Val or Arg, and X₁₂ is Leu, Gln, His, Ile, Lys, or Ser, and the sialidase comprises at least one mutation relative to wild-type human Neu2 (SEQ ID NO: 2). In certain embodiments, X₁ is Ala, Asp, Met, or not present, X₂ is Tyr or Val, X₃ is Lys or Asp, X₄ is Pro, Asn, Gly, Ser or Thr, X₅ is Ala or Glu, X₆ is Ile or Lys, X₇ is Gln or Ala, X₈ is Ser or Arg, X₉ is Trp or Lys, X₁₀ is Ala or Cys, X₁₁ is Val or Arg, and X₁₂ is Leu or Ile.

In certain embodiments, the recombinant mutant human sialidase comprises a conservative substitution relative to a recombinant mutant human sialidase sequence disclosed herein. As used herein, the term “conservative substitution” refers to a substitution with a structurally similar amino acid. For example, conservative substitutions may include those within the following groups: Ser and Cys; Leu, Ile, and Val; Glu and Asp; Lys and Arg; Phe, Tyr, and Trp; and Gln, Asn, Glu, Asp, and His. Conservative substitutions may also be defined by the BLAST (Basic Local Alignment Search Tool) algorithm, the BLOSUM substitution matrix (e.g., BLOSUM 62 matrix), or the PAM substitution:p matrix (e.g., the PAM 250 matrix).

### Sialidase Fusion Proteins/Antibody Conjugates

In certain embodiments, a sialidase is fused to a portion or fragment of an antibody, such as an immunoglobulin Fc domain (also referred to herein as an Fc domain), or an immunoglobulin antigen-binding domain (also referred to herein as an antigen-binding domain). In certain embodiments, the sialidase and antibody or portion thereof (e.g., immunoglobulin Fc domain or antigen-binding domain) are linked by a peptide bond or an amino acid linker.

As used herein, unless otherwise indicated, the term “fusion protein” is understood to refer to a single polypeptide chain comprising amino acid sequences based upon two or more separate proteins or polypeptide chains, where the two amino acid sequences may be fused together directly or via an intervening linker sequence, e.g., via an intervening amino acid linker. A nucleotide sequence encoding a fusion protein can, for example, be created using conventional recombinant DNA technologies.

In certain embodiments, the fusion protein comprises a tag, such as a Strep tag (e.g., a Strep II tag), a His tag (e.g., a 10× His tag (SEQ ID NO: 157)), a myc tag, or a FLAG tag. The tag can be located on the C-terminus or the N-terminus of the fusion protein. In certain embodiments, a fusion protein comprises a sialidase portion joined to a polypeptide comprising an immunoglobulin heavy chain in an N- to C-terminal orientation, wherein the sialidase portion comprises an N-terminal addition of MEDLRP (SEQ ID NO: 17), and a Strep II Tag is located on the C-terminus of the immunoglobulin heavy chain or the N-terminus of the sialidase portion.

The sialidase portion of a fusion protein described herein can be any sialidase disclosed herein, e.g., a fungal, bacterial, non-human mammalian or human sialidase. In certain embodiments, the sialidase portion is a sialidase comprising at least one mutation relative to a wild-type human sialidase, e.g., a substitution, deletion, or addition of at least one amino acid, as described above.

As used herein, unless otherwise indicated, the term “antibody” is understood to mean an intact antibody (e.g., an intact monoclonal antibody), or a fragment thereof, such as a Fc fragment of an antibody (e.g., an Fc fragment of a monoclonal antibody), or an antigen-binding fragment of an antibody (e.g., an antigen-binding fragment of a monoclonal antibody), including an intact antibody, antigen-binding fragment, or Fc fragment that has been modified, engineered, or chemically conjugated. Examples of antigen-binding fragments include Fab, Fab′, (Fab′)₂, Fv, single chain antibodies (e.g., scFv), minibodies, and diabodies. Examples of antibodies that have been modified or engineered include chimeric antibodies, humanized antibodies, and multispecific antibodies (e.g., bispecific antibodies). An example of a chemically conjugated antibody is an antibody conjugated to a toxin moiety.

In certain embodiments, the fusion protein comprises an immunoglobulin Fc domain. As used herein, unless otherwise indicated, the term “immunoglobulin Fc domain” refers to a fragment of an immunoglobulin heavy chain constant region which, either alone or in combination with a second immunoglobulin Fc domain, is capable of binding to an Fc receptor. An immunoglobulin Fc domain may include, e.g., immunoglobulin CH2 and CH3 domains. An immunoglobulin Fc domain may include, e.g., immunoglobulin CH2 and CH3 domains and an immunoglobulin hinge region. Boundaries between immunoglobulin hinge regions, CH2, and CH3 domains are well known in the art, and can be found, e.g., in the PROSITE database (available on the world wide web at prosite.expasy.org).

In certain embodiments, the immunoglobulin Fc domain is derived from a human IgG1, IgG2, IgG3, IgG4, IgA1, IgA2, IgD, IgE, and IgM Fc domain. A single amino acid substitution (S228P according to Kabat numbering; designated IgG4Pro) may be introduced to abolish the heterogeneity observed in recombinant IgG4 antibody. See Angal, S. et al. (1993) MOL. IMMUNOL. 30:105-108.

In certain embodiments, the immunoglobulin Fc domain is derived from a human IgG1 isotype or another isotype that elicits antibody-dependent cell-mediated cytotoxicity (ADCC) and/or complement mediated cytotoxicity (CDC). In certain embodiments, the immunoglobulin Fc domain is derived from a human IgG1 isotype (e.g., SEQ ID NO: 29 or SEQ ID NO: 62).

In certain embodiments, the immunoglobulin Fc domain is derived from a human IgG4 isotype or another isotype that elicits little or no antibody-dependent cell-mediated cytotoxicity (ADCC) and/or complement mediated cytotoxicity (CDC). In certain embodiments, the immunoglobulin Fc domain is derived from a human IgG4 isotype.

In certain embodiments, the immunoglobulin Fc domain comprises either a “knob” mutation, e.g., T366Y or a “hole” mutation, e.g., Y407T for heterodimerization with a second polypeptide (residue numbers according to EU numbering, Kabat, E. A., et al. (1991) SEQUENCES OF PROTEINS OF IMMUNOLOGICAL INTEREST, FIFTH EDITION, U.S. Department of Health and Human Services, NIH Publication No. 91-3242).

In certain embodiments, the fusion protein comprises an immunoglobulin antigen-binding domain. The inclusion of such a domain may improve targeting of a fusion protein to a sialylated cancer cell and/or to the tumor microenvironment. As used herein, unless otherwise indicated, the term “immunoglobulin antigen-binding domain” refers to a polypeptide that, alone or in combination with another immunoglobulin antigen-binding domain, defines an antigen-binding site. Exemplary immunoglobulin antigen-binding domains include, for example, immunoglobulin heavy chain variable region and an immunoglobulin light chain variable region, where the variable regions together define an antigen binding site.

The immunoglobulin antigen-binding domain and/or antigen binding site can be derived from an antibody selected from, for example, adecatumumab, ascrinvacumab, cixutumumab, conatumumab, daratumumab, drozitumab, duligotumab, durvalumab, dusigitumab, enfortumab, enoticumab, epratuxumab, figitumumab, ganitumab, glembatumumab, intetumumab, ipilimumab, iratumumab, icrucumab, lexatumumab, lucatumumab, mapatumumab, narnatumab, necitumumab, nesvacumab, ofatumumab, olaratumab, panitumumab, patritumab, pritumumab, radretumab, ramucirumab, rilotumumab, robatumumab, seribantumab, tarextumab, teprotumumab, tovetumab, vantictumab, vesencumab, votumumab, zalutumumab, flanvotumab, altumomab, anatumomab, arcitumomab, bectumomab, blinatumomab, detumomab, ibritumomab, minretumomab, mitumomab, moxetumomab, naptumomab, nofetumomab, pemtumomab, pintumomab, racotumomab, satumomab, solitomab, taplitumomab, tenatumomab, tositumomab, tremelimumab, abagovomab, atezolizumab, durvalumab, avelumab, igovomab, oregovomab, capromab, edrecolomab, nacolomab, amatuximab, bavituximab, brentuximab, cetuximab, derlotuximab, dinutuximab, ensituximab, futuximab, girentuximab, indatuximab, isatuximab, margetuximab, rituximab, siltuximab, ublituximab, ecromeximab, abituzumab, alemtuzumab, bevacizumab, bivatuzumab, brontictuzumab, cantuzumab, cantuzumab, citatuzumab, clivatuzumab, dacetuzumab, demcizumab, dalotuzumab, denintuzumab, elotuzumab, emactuzumab, emibetuzumab, enoblituzumab, etaracizumab, farletuzumab, ficlatuzumab, gemtuzumab, imgatuzumab, inotuzumab, labetuzumab, lifastuzumab, lintuzumab, lirilumab, lorvotuzumab, lumretuzumab, matuzumab, milatuzumab, moxetumomab, nimotuzumab, obinutuzumab, ocaratuzumab, otlertuzumab, onartuzumab, oportuzumab, parsatuzumab, pertuzumab, pidilizumab, pinatuzumab, polatuzumab, sibrotuzumab, simtuzumab, tacatuzumab, tigatuzumab, trastuzumab, tucotuzumab, urelumab, vandortuzumab, vanucizumab, veltuzumab, vorsetuzumab, sofituzumab, catumaxomab, ertumaxomab, depatuxizumab, ontuxizumab, blontuvetmab, tamtuvetmab, nivolumab, pembrolizumab, epratuzumab, MEDI9447, urelumab, utomilumab, hu3F8, hu14.18-IL-2, 3F8/OKT3BsAb, lirilumab, BMS-986016 pidilizumab, AMP-224, AMP-514, BMS-936559, atezolizumab, and avelumab. In certain embodiments, the immunoglobulin antigen-binding domain can be derived from an antibody selected from trastuzumab, cetuximab, daratumumab, girentuximab, panitumumab, ofatumumab, and rituximab.

In certain embodiments, the immunoglobulin antigen-binding domain is derived from trastuzumab. The trastuzumab heavy chain amino acid sequence is depicted in SEQ ID NO: 40, and the trastuzumab light chain amino acid sequence is depicted in SEQ ID NO: 41. The amino acid sequence of an exemplary scFv derived from trastuzumab is depicted in SEQ ID NO: 42.

The immunoglobulin antigen-binding domain and/or antigen binding site can be derived from an antibody that binds a cancer antigen selected from, for example, adenosine A2a receptor (A2aR), A kinase anchor protein 4 (AKAP4), B melanoma antigen (BAGE), brother of the regulator of imprinted sites (BORIS), breakpoint cluster region Abelson tyrosine kinase (BCR/ABL), CA125, CAIX, CD19, CD20, CD22, CD30, CD33, CD52, CD73, CD137, carcinoembryonic antigen (CEA), CS1, cytotoxic T-lymphocyte-associated antigen 4 (CTLA-4), estrogen receptor binding site associated antigen 9 (EBAG9), epidermal growth factor (EGF), epidermal growth factor receptor (EGFR), epithelial cell adhesion molecule (EpCAM) (17-1A), FR-alpha, G antigen (GAGE), disialoganglioside GD2 (GD2), glycoprotein 100 (gp100), human epidermal growth factor receptor 2 (Her2), hepatocyte growth factor (HGF), human papillomavirus 16 (HPV-16), heat-shock protein 105 (HSP105), isocitrate dehydrogenase type 1 (IDH1), idiotype (NeuGcGM3), indoleamine-2,3-dioxygenase 1 (IDO1), IGF-1, IGF1R, IGG1K, killer cell immunoglobulin-like receptor (KIR), lymphocyte activation gene 3 (LAG-3), lymphocyte antigen 6 complex K (LY6K), melanoma antigen 3 (MAGE-A3), melanoma antigen C2 (MAGE-C2), melanoma antigen D4 (MAGE-D4), melanoma antigen recognized by T-cells 1 (Melan-A/MART-1), N-methyl-N′-nitroso-guanidine human osteosarcoma transforming gene (MET), mucin 1 (MUC1), mucin 4 (MUC4), mucin 16 (MUC16), New York esophageal squamous cell carcinoma 1 (NY-ESO-1), prostatic acid phosphatase (PAP), programmed cell death receptor 1 (PD-1), programmed cell death receptor ligand 1 (PD-L1), phosphatidylserine, preferentially expressed antigen of melanoma (PRAME), prostate specific antigen (PSA), receptor tyrosine kinase orphan receptor 1 (ROR1), scatter factor receptor kinase, sialyl-Tn, sperm-associated antigen 9 (SPAG-9), synovial sarcoma X-chromosome breakpoint 1 (SSX1), survivin, telomerase, T-cell immunoglobulin domain and mucin domain-3 (TIM-3), vascular endothelial growth factor (VEGF) (e.g., VEGF-A), vascular endothelial growth factor Receptor 2 (VEGFR2), V-domain immunoglobulin-containing suppressor of T-cell activation (VISTA), Wilms' Tumor-1 (WT1), X chromosome antigen 1b (XAGE-1b), 5T4, Mesothelin, Glypican 3 (GPC3), Folate Receptor α (FRα), Prostate Specific Membrane Antigen (PSMA), cMET, CD38, B Cell Maturation Antigen (BCMA), CD123, Siglec7 and Siglec9.

The disclosure further provides antibody conjugates containing one or more of the fusion proteins disclosed herein. As used herein, unless otherwise indicated, the term “antibody conjugate” is understood to refer to an antibody, or a functional fragment thereof, that comprises antigen-binding activity and/or Fc receptor-binding activity, conjugated (e.g., covalently coupled) to an additional functional moiety. In certain embodiments, the antibody or functional antibody fragment is conjugated to a sialidase enzyme, e.g., a sialidase enzyme disclosed herein. In certain embodiments, an antibody conjugate comprises a single polypeptide chain. In certain embodiments, an antibody conjugate comprises two, three, four, or more polypeptide chains that are covalently or non-covalently associated together to produce a multimeric complex, e.g., a dimeric, trimeric or tetrameric complex.

TABLE 1 shows antibodies and antibody-drug conjugates suitable for use in accordance with the present invention, the antigen bound by the antibody or antibody-drug conjugate, and for certain antibodies, the type of cancer targeted by the antibody or antibody-drug conjugate.

TABLE 1

| Antibody or antibody- | | |
|---|---|---|
| drug conjugate | Cancer Antigen | Cancer Type |
| oregovomab | CA125 | |
| girentuximab | CAIX | |
| obinutuzumab | CD20 | |
| ofatumumab | CD20 | |
| rituximab | CD20 | |
| alemtuzumab | CD52 | |
| ipilimumab | cytotoxic T-lymphocyte-associated | |
| | antigen 4 (CTLA-4) | |
| tremelimumab | CTLA-4 | |
| Cetuximab | epidermal growth factor receptor | |
| | (EGFR) | |
| necitumumab | EGFR | |
| panitumumab | EGFR | |
| zalutumumab | EGFR | |
| edrecolomab | epithelial cell adhesion molecule | |
| | (EpCAM) (17-1A) | |
| farletuzumab | FR-alpha | |
| pertuzumab | human epidermal growth factor | |
| | receptor 2 (Her2) | |
| trastuzumab | Her2 | |
| rilotumumab | HGF | |
| figitumumab | IGF-1 | |
| Ganitumab | IGF1R | |
| durvalumab | IGG1K | |
| bavituximab | Phosphatidylserine | |
| onartuzumab | scatter factor receptor kinase | |
| bevacizumab | vascular endothelial growth factor-A | |
| | (VEGF-A) | |
| ramucirumab | vascular endothelial growth factor | |
| | Receptor 2 (VEGFR2) | |
| blinatumomab | CD19 | acute lymphoblastic leukemia |
| | | (ALL) |
| Rituximab; | CD20 | non-Hodgkin's lymphoma |
| ofatumumab; | | (NHL), chronic lymphocytic |
| ibritumomab (e.g., ⁹⁰Y- | | leukemia (CLL) |
| ibritumomab; | | B-cell NHL |
| tositumomab (e.g., ¹³¹I- | | pre-B ALL |
| tositumomab | | |
| brentuximab (e.g., | CD30 | Hodgkin's lymphoma |
| brentuximab vedotin | | |
| gemtuzumab (e.g., | CD33 | acute myelogenous leukemia |
| gemtuzumab ozogamicin | | (AML) |
| Alemtuzumab | CD52 | CLL |
| Ipilimumab | cytotoxic T-lymphocyte-associated | Unresectable or metastatic |
| | antigen 4 (CTLA-4) | melanoma |
| cetuximab; | epidermal growth factor receptor | colorectal cancer (CRC) |
| panitumumab | (EGFR) | Head and Neck |
| Catumaxomab | epithelial cell adhesion molecule | Malignant ascites |
| | (EpCAM) | |
| trastuzumab; | human epidermal growth factor | Breast |
| pertuzumab | receptor 2 (HER2) | |
| nivolumab; | programmed cell death receptor 1 | Metastatic melanoma, non- |
| pembrolizumab | (PD-1) | small cell lung cancer |
| | | (NSCLC) |
| Bevacizumab | vascular endothelial growth factor | Breast, Cervical |
| | (VEGF) | CRC, NSCLC |
| | | renal cell carcinoma (RCC), |
| | | Ovarian |
| | | Glioblastoma |
| Ramucirumab | vascular endothelial growth factor | Gastric |
| | receptor 2 (VEGF-R2) | NSCLC |
| Epratuzumab; | CD22 | acute lymphoblastic leukemia |
| moxetumomab; | | (ALL) |
| inotuzumab (e.g., | | |
| inotuzumab ozogamicin) | | |
| MEDI9447 | CD73 | Advanced solid tumors |
| Urelumab; | CD137 | Advanced solid tumors |
| utomilumab (PF- | | |
| 05082566) | | |
| Elotuzumab | CD2 subset 1 (CS1) | Multiple myeloma |
| Tremelimumab | cytotoxic T-lymphocyte-associated | Malignant mesothelioma |
| | antigen 4 (CTLA-4) | |
| Necitumumab | epidermal growth factor receptor | non-small cell lung cancer |
| | (EGFR) | (NSCLC) |
| dinutuximab; hu3F8; | disialoganglioside GD2 (GD2) | Neuroblastoma |
| hu14.18-IL-2; | | Retinoblastoma |
| 3F8/OKT3BsAb | | Melanoma other solid tumors |
| Racotumomab | Idiotype (NeuGcGM3) | NSCLC, Breast |
| | | Melanoma |
| Lirilumab | killer cell immunoglobulin-like | Lymphoma |
| | receptor (KIR) | |
| BMS-986016 | lymphocyte activation gene 3 (LAG- | Breast, Hematological, |
| | 3) | Advanced solid tumors |
| Onartuzumab | N-methyl-N′-nitroso-guanidine | NSCLC |
| | human osteosarcoma transforming | |
| | gene (MET) | |
| abagovomab; | mucin 16 (MUC16) | Ovarian |
| oregovomab | | |
| pidilizumab; | programmed cell death receptor 1 | B-cell lymphoma |
| AMP-224; AMP-514 | (PD-1) | Melanoma, CRC |
| BMS-936559; | programmed cell death receptor | NSCLC, renal cell carcinoma |
| atezolizumab; | ligand 1 (PD-L1) | (RCC) |
| durvalumab; avelumab | | Bladder, Breast |
| | | Melanoma, squamous cell |
| | | carcinoma of the head and |
| | | neck (SCCHN) |
| naptumomab (e.g., | 5T4 | RCC, CRC |
| naptumomab estafenatox | | Prostate |

In certain embodiments, the sialidase portion of the fusion protein can be linked or fused directly to the antibody portion (e.g., immunoglobulin Fc domain and/or immunoglobulin antigen-binding domain) of the fusion protein. In other embodiments, the sialidase portion can be covalently bound to the antibody portion by a linker.

The linker may couple, with one or more natural amino acids, the sialidase, or functional fragment thereof, and the antibody portions or fragments, where the amino acid (for example, a cysteine amino acid) may be introduced by site-directed mutagenesis. The linker may include one or more unnatural amino acids. It is contemplated that, in certain circumstances, a linker containing for example, one or more sulfhydryl reactive groups (e.g., a maleimide) may covalently link a cysteine in the sialidase portion or the antibody portion that is a naturally occurring cysteine residue or is the product of site-specific mutagenesis.

The linker may be a cleavable linker or a non-cleavable linker. Optionally or in addition, the linker may be a flexible linker or an inflexible linker.

The linker should be a length sufficiently long to allow the sialidase and the antibody portions to be linked without steric hindrance from one another and sufficiently short to retain the intended activity of the fusion protein. The linker preferably is sufficiently hydrophilic to avoid or minimize instability of the fusion protein. The linker preferably is sufficiently hydrophilic to avoid or minimize insolubility of the fusion protein. The linker should be sufficiently stable in vivo (e.g., it is not cleaved by serum, enzymes, etc.) to permit the fusion protein to be operative in vivo.

The linker may be from about 1 angstroms (Å) to about 150 Å in length, or from about 1 Å to about 120 Å in length, or from about 5 Å to about 110 Å in length, or from about 10 Å to about 100 Å in length. The linker may be greater than about 2, 3, 4, 5, 6, 7, 8, 9, 10, 11, 12, 13, 14, 15, 16, 17, 18, 19, 20, 25, 27, 30 or greater angstroms in length and/or less than about 110, 100, 90, 85, 80, 75, 70, 65, 60, 55, 50, 45, 43, 42, 41, 40, 39, 38, 37, 36, 35, 34, 33, 32, 31, or fewer A in length. Furthermore, the linker may be about 5, 10, 15, 20, 25, 30, 35, 40, 45, 50, 55, 60, 65, 70, 75, 80, 85, 90, 95, 100, 110, and 120 Å in length.

In certain embodiments, the linker comprises a polypeptide linker that connects or fuses the sialidase portion of the fusion protein to the antibody portion (e.g., immunoglobulin Fc domain and/or immunoglobulin antigen-binding domain) of the fusion protein. For example, it is contemplated that a gene encoding a sialidase portion linked directly or indirectly (for example, via an amino acid containing linker) to an antibody portion can be created and expressed using conventional recombinant DNA technologies. For example, the amino terminus of a sialidase portion can be linked to the carboxy terminus of either the light or the heavy chain of an antibody portion. For example, for a Fab fragment, the amino terminus or carboxy terminus of the sialidase can be linked to the first constant domain of the heavy antibody chain (CH1). When a linker is employed, the linker may comprise hydrophilic amino acid residues, such as Gln, Ser, Gly, Glu, Pro, His and Arg. In certain embodiments, the linker is a peptide containing 1-25 amino acid residues, 1-20 amino acid residues, 2-15 amino acid residues, 3-10 amino acid residues, 3-7 amino acid residues, 4-25 amino acid residues, 4-20 amino acid residues, 4-15 amino acid residues, 4-10 amino acid residues, 5-25 amino acid residues, 5-20 amino acid residues, 5-15 amino acid residues, or 5-10 amino acid residues. Exemplary linkers include glycine and serine-rich linkers, e.g., (GlyGlyPro)ₙ (SEQ ID NO: 158), or (GlyGlyGlyGlySer)ₙ (SEQ ID NO: 159), where n is 1-5. In certain embodiments, the linker is (Gly₄Ser)₂ (SEQ ID NO: 160). Additional exemplary linker sequences are disclosed, e.g., in George et al. (2003) Protein Engineering 15:871-879, and U.S. Pat. Nos. 5,482,858 and 5,525,491.

In certain embodiments, the fusion protein comprises any one of SEQ ID NOs: 43-54, 67-72, 90-96, 99-105, or 108-155, or an amino acid sequence that has at least 85%, 90%, 95%, 96%, 97%, 98%, or 99% sequence identity to any one of SEQ ID NOs: 43-54, 67-72, 90-96, 99-105, or 108-155.

In certain embodiments, a sialidase for use in the invention is an antibody conjugate comprising a fusion protein disclosed herein. The antibody conjugate may comprise a single polypeptide chain (i.e., a fusion protein disclosed herein) or, the antibody conjugate may comprise additional polypeptide chains (e.g., one, two, or three additional polypeptide chains). For example, an antibody conjugate may comprise a first polypeptide (fusion protein) comprising a sialidase enzyme and an immunoglobulin heavy chain, and a second polypeptide comprising an immunoglobulin light chain, where, for example, the immunoglobulin heavy and light chains together define a single antigen-binding site.

In certain embodiments, the antibody conjugate can include a single sialidase. In other embodiments, the antibody conjugate can include more than one (e.g., two) sialidases. If more than one sialidase is included, the sialidases can be the same or different. In certain embodiments, the antibody conjugate can include a single antigen-binding site. In other embodiments, the antibody conjugate can include more than one (e.g., two) antigen-binding sites. If two antigen-binding sites are used, they can be the same or different. In certain embodiments, the antibody conjugate comprises an immunoglobulin Fc fragment.

In certain embodiments, the antibody conjugate comprises one or two immunoglobulin heavy chains, or a functional fragment thereof. In certain embodiments, the antibody conjugate comprises one or two immunoglobulin light chains, or a functional fragment thereof. In certain embodiments, the antibody conjugate comprises a sialidase fused to the N- or C-terminus of an immunoglobulin heavy chain or an immunoglobulin light chain.

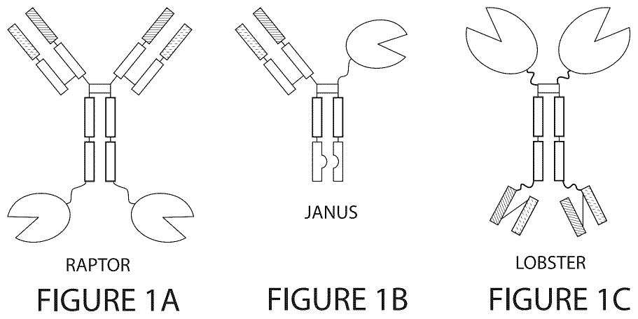

In certain embodiments, the antibody conjugate comprises a first polypeptide comprising a first immunoglobulin light chain; a second polypeptide comprising a first immunoglobulin heavy chain and a first sialidase; a third polypeptide comprising a second immunoglobulin heavy chain and a second sialidase; and a fourth polypeptide comprising a second immunoglobulin light chain. An example of this embodiment is shown in FIG. 1A. The first and second polypeptides can be covalently linked together, the third and fourth polypeptides can be covalently linked together, and the second and third polypeptides can be covalently linked together. The covalent linkages can be disulfide bonds. In certain embodiments, the first polypeptide and the second polypeptide together define a first antigen-binding site, and the third polypeptide and the fourth polypeptide together define a second antigen-binding site. In certain embodiments, the second and third polypeptides comprise the first and second immunoglobulin heavy chain and the first and second sialidase, respectively, in an N- to C-terminal orientation. In certain embodiments, the second and third polypeptides comprise the first and second sialidase and the first and second immunoglobulin heavy chain, respectively, in an N- to C-terminal orientation.

In certain embodiments, the antibody conjugate comprises a first polypeptide comprising an immunoglobulin light chain; a second polypeptide comprising an immunoglobulin heavy chain; and a third polypeptide comprising an immunoglobulin Fc domain and a sialidase. An example of this embodiment is shown in FIG. 1B. The first and second polypeptides can be covalently linked together and the second and third polypeptides can be covalently linked together. The covalent linkages can be disulfide bonds. In certain embodiments, the first polypeptide and the second polypeptide together define an antigen-binding site. In certain embodiments, the third polypeptide comprises the sialidase and the immunoglobulin Fc domain in an N- to C-terminal orientation or the immunoglobulin Fc domain and the sialidase in an N- to C-terminal orientation.

In certain embodiments, the first polypeptide comprises the amino acid sequence of SEQ ID NO: 49, or an amino acid sequence that has at least 85%, 90%, 95%, 96%, 97%, 98%, or 99% sequence identity to SEQ ID NO: 49. In certain embodiments, the second polypeptide comprises the amino acid sequence of SEQ ID NO: 50 or an amino acid sequence that has at least 85%, 90%, 95%, 96%, 97%, 98%, or 99% sequence identity to SEQ ID NO: 50. In certain embodiments, the third polypeptide comprises the amino acid sequence of SEQ ID NO: 51, SEQ ID NO: 52, SEQ ID NO: 53, SEQ ID NO: 54, SEQ ID NO: 69, SEQ ID NO: 70, SEQ ID NO: 71, SEQ ID NO: 72, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 108, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, or SEQ ID NO: 125, or an amino acid sequence that has at least 85%, 90%, 95%, 96%, 97%, 98%, or 99% sequence identity to SEQ ID NO: 51, SEQ ID NO: 52, SEQ ID NO: 53, SEQ ID NO: 54, SEQ ID NO: 69, SEQ ID NO: 70, SEQ ID NO: 71, SEQ ID NO: 72, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 108, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, or SEQ ID NO: 125.

In certain embodiments, the third polypeptide comprises the amino acid sequence of

X₁X₂SX₃PX₄LQKESVFQSGAHAYRIPALLYLPGQQSLLAFAEQRASKKDE
HAELIVLRRGDYDAPTHQVQWQAQEVVAQARLDGHRSMNPCPLYDAQTG
TLFLFFIAIPGQVTEQQQLQTRANVTRLX₅QVTSTDHGRTWSSPRDLTD
AAIGPAYREWSTFAVGPGHCLQLHDRARSLVVPAYAYRKLHPX₆QRPIP
SAFX₇FLSHDHGRTWARGHFVAQDTLECQVAEVETGEQRVVTLNARSHLR
ARVQAQSTNDGLDFQX₈SQLVKKLVEPPPQGX₉QGSVISFPSPRSGPGSP
AQWLLYTHPTHSWQRADLGAYLNPRPPAPEAWSEPX₁₀LLAKGSX₁₁AYS
DLQSMGTGPDGSPLFGX₁₂LYEANDYEEIVFLMFTLKQAFPAEYLPQGGG
GSGGGGSDKTHTCPPCPAPELLGGPSVFLFPPKPKDTLMISRTPEVTCV
VVDVSHEDPEVKFNWYVDGVEVHNAKTKPREEQYNSTYRVVSVLTVLHQ
DWLNGKEYKCKVSNKALPAPIEKTISKAKGQPREPQVYTLPPSREEMTK
NQVSLTCLVKGFYPSDIAVEWESNGQPENNYKTTPPVLDSDGSFFLTSK
LTVDKSRWQQGNVFSCSVMHEALHNHYTQKSLSLSPGK

(SEQ ID NO: 77), wherein X₁ is Ala, Arg, Asn, Asp, Gln, Glu, Gly, His, Leu, Lys, Met, Phe, Thr, Val, or not present, X₂ is Ala or Lys, X₃ is Asn or Leu, X₄ is Phe, Trp, Tyr or Val, X₅ is Ala, Cys, Ile, Ser, or Val, X₆ is Arg, Ile, or Lys, X₇ is Ala, Cys, Leu, or Val, X₈ is Glu or Lys, X₉ is Cys or Val, X₁₀ is Lys or Val, X₁₁ is Ala, Cys, Ser, or Val, and X₁₂ is Cys, Leu, or Val.

In certain embodiments, the third polypeptide comprises the amino acid sequence of

X₁ASLPX₂LQKESVFQSGAHAYRIPALLYLPGQQSLLAFAEQRASKKDEH
AELIVLRRGDYDAPTHQVQWQAQEVVAQARLDGHRSMNPCPLYDAQTGT
LFLFFIAIPGQVTEQQQLQTRANVTRLCQVTSTDHGRTWSSPRDLTDAA
IGPAYREWSTFAVGPGHCLQLHDRARSLVVPAYAYRKLHPX₃QRPIPSA
FCFLSHDHGRTWARGHFVAQDTLECQVAEVETGEQRVVTLNARSHLRAR
VQAQSTNDGLDFQESQLVKKLVEPPPQGCQGSVISFPSPRSGPGSPAQW
LLYTHPTHSWQRADLGAYLNPRPPAPEAWSEPVLLAKGSX₄AYSDLQSM
GTGPDGSPLFGCLYEANDYEEIVFLMFTLKQAFPAEYLPQGGGGSGGGG
SDKTHTCPPCPAPELLGGPSVFLFPPKPKDTLMISRTPEVTCVVVDVSH
EDPEVKFNWYVDGVEVHNAKTKPREEQYNSTYRVVSVLTVLHQDWLNGK
EYKCKVSNKALPAPIEKTISKAKGQPREPQVYTLPPSREEMTKNQVSLT
CLVKGFYPSDIAVEWESNGQPENNYKTTPPVLDSDGSFFLTSKLTVDKS
RWQQGNVFSCSVMHEALHNHYTQKSLSLSPGK

(SEQ ID NO: 74), wherein X₁ is Ala, Asn, Asp, His, Leu, Met, Phe, Thr, or not present, X₂ is Phe, Trp, Tyr or Val, X₃ is Arg, Ile, or Lys, and X₄ is Ala, Cys, Ser, or Val. In certain embodiments, X₁ is Ala, Asp, Met, or not present, X₂ is Tyr or Val, X₃ is Ile or Lys, and X₄ is Ala or Cys.

In certain embodiments, the third polypeptide comprises the amino acid sequence of

X₁X₂SX₃X₄X₅LQX₆ESVFQSGAHAYRIPALLYLPGQQSLLAFAEQRASX₇
X₈DEHAELIVX₉RRGDYDAX₁₀THQVQWX₁₁AQEVVAQAX₁₂LDGHRSMNP
CPLYDX₁₃QTGTLFLFFIAIPX₁₄X₁₅VTEX₁₆QQLQTRANVTRLX₁₇X₁₈V
TSTDHGRTWSSPRDLTDAAIGPX₁₉YREWSTFAVGPGHX₂₀LQLHDRX₂₁
RSLVVPAYAYRKLHPX₂₂QRPIPSAFX₂₃FLSHDHGRTWARGHFVAQDT
X₂₄ECQVAEVETGEQRVVTLNARSHLRARVQAQSX₂₅NX₂₆GLDFQX₂₇S
QLVKKLVEPPPX₂₈GX₂₉QGSVISFPSPRSGPGSPAQX₃₀LLYTHPTH
X₃₁X₃₂QRADLGAYLNPRPPAPEAWSEPX₃₃LLAKGSX₃₄AYSDLQSMGT
GPDGSPLFGX₃₅LYEANDYEEIX₃₆FX₃₇MFTLKQAFPAEYLPQX₃₈DKT
HTCPPCPAPELLGGPSVFLFPPKPKDTLMISRTPEVTCVVVDVSHED
PEVKFNWYVDGVEVHNAKTKPREEQYNSTYRVVSVLTVLHQDWLNGK
EYKCKVSNKALPAPIEKTISKAKGQPREPQVYTLPPSREEMTKNQVS
LTCLVKGFYPSDIAVEWESNGQPENNYKTTPPVLDSDGSFFLTSKLT
VDKSRWQQGNVFSCSVMHEALHNHYTQKSLSLSPGK

(SEQ ID NO: 98), wherein X₁ is Ala, Arg, Asn, Asp, Gln, Glu, Gly, His, Leu, Lys, Met, Phe, Thr, Val, or not present, X₂ is Ala or Lys, X₃ is Asn or Leu, X₄ is Pro or His, X₅ is Phe, Trp, Tyr or Val, X₆ is Lys or Asp. X₇ is Lys, Arg, or Glu. X₈ is Lys, Ala, Arg, or Glu, X₉ is Leu or Met, X₁₀ is Pro, Asn, Asp, His, Glu, Gly, Ser or Thr, X₁₁ is Gln or His, X₁₂ is Arg or Lys, X₁₃ is Ala, Glu or Lys, X₁₄ is Gly or Asp, X₁₅ is Gln or His, X₁₆ is Gln, Arg, or Lys, X₁₇ is Ala, Cys, Ile, Ser, Val, or Leu, X₁₈ is Gln or Leu, X₁₉ is Ala or Val, X₂₀ is Cys or Gly, X₂₁ is Ala or Gly, X₂₂ is Arg, Ile, or Lys, X₂₃ is Ala, Cys, Leu, or Val, X₂₄ is Leu, Ala, or Val, X₂₅ is Thr or Ala, X₂₆ is Asp or Gly, X₂₇ is Glu or Lys, X₂₈ is Gln, Ala, His, Phe, or Pro, X₂₉ is Cys or Val, X₃₀ is Trp or Arg, X₃₁ is Ser or Arg, X₃₂ is Trp or Lys, X₃₃ is Lys or Val, X₃₄ is Ala, Cys, Ser, or Val, X₃₅ is Cys, Leu, or Val, X₃₅ is Val or Arg, X₃₇ is Leu, Gln, His, Ile, Lys, or Ser, and X₃₈ is GGGGSGGGGS or EPKSS, and the sialidase comprises at least one mutation relative to wild-type human Neu2 (SEQ ID NO: 2).

In certain embodiments, the third polypeptide comprises the amino acid sequence of

X₁ASLPX₂LQX₃ESVFQSGAHAYRIPALLYLPGQQSLLAFAEQRASKKDE
HAELIVLRRGDYDAX₄THQVQWQAQEVVAQARLDGHRSMNPCPLYDX₅Q
TGTLFLFFIAIPGQVTEQQQLQTRANVTRLCQVTSTDHGRTWSSPRDL
TDAAIGPAYREWSTFAVGPGHCLQLHDRARSLVVPAYAYRKLHPX₆QRP
IPSAFCFLSHDHGRTWARGHFVAQDTLECQVAEVETGEQRVVTLNARSH
LRARVQAQSTNDGLDFQESQLVKKLVEPPPX₇GCQGSVISFPSPRSGPG
SPAQWLLYTHPTHX₈X₉QRADLGAYLNPRPPAPEAWSEPVLLAKGSX₁₀A
YSDLQSMGTGPDGSPLFGCLYEANDYEEIX₁₁FX₁₂MFTLKQAFPAEYLP
QX₁₃DKTHTCPPCPAPELLGGPSVFLFPPKPKDTLMISRTPEVTCVVVD
VSHEDPEVKFNWYVDGVEVHNAKTKPREEQYNSTYRVVSVLTVLHQDW
LNGKEYKCKVSNKALPAPIEKTISKAKGQPREPQVYTLPPSREEMTKN
QVSLTCLVKGFYPSDIAVEWESNGQPENNYKTTPPVLDSDGSFFLTSK
LTVDKSRWQQGNVFSCSVMHEALHNHYTQKSLSLSPGK

(SEQ ID NO: 97), wherein X₁ is Ala, Arg, Asn, Asp, Gln, Glu, Gly, His, Leu, Lys, Met, Phe, Thr, Val, or not present, X₂ is Phe, Trp, Tyr or Val, X₃ is Lys or Asp, X₄ is Pro, Asn, Asp, His, Glu, Gly, Ser or Thr, X₅ is Ala, Glu, or Lys, X₆ is Arg, Ile, or Lys, X₇ is Gln, Ala, His, Phe, or Pro, X₈ is Ser or Arg, X₉ is Trp or Lys, X₁₀ is Ala, Cys, Ser, or Val, X₁₁ is Val or Arg, X₁₂ is Leu, Gln, His, Ile, Lys, or Ser, and X₁₃ is GGGGSGGGGS or EPKSS, and the sialidase comprises at least one mutation relative to wild-type human Neu2 (SEQ ID NO: 2). In certain embodiments, X₁ is Ala, Asp, Met, or not present, X₂ is Tyr or Val, X₃ is Lys or Asp, X₄ is Pro, Asn, Gly, Ser or Thr, X₅ is Ala or Glu, X₆ is Ile or Lys, X₇ is Gln or Ala, X₈ is Ser or Arg, X₉ is Trp or Lys, X₁₀ is Ala or Cys, X₁₁ is Val or Arg, and X₁₂ is Leu or Ile.

In certain embodiments, the first polypeptide comprises SEQ ID NO: 49, the second polypeptide comprises SEQ ID NO: 50, and the third polypeptide comprises SEQ ID NO: 51. In certain embodiments, the first polypeptide comprises SEQ ID NO: 49, the second polypeptide comprises SEQ ID NO: 50, and the third polypeptide comprises SEQ ID NO: 52. In certain embodiments, the first polypeptide comprises SEQ ID NO: 49, the second polypeptide comprises SEQ ID NO: 50, and the third polypeptide comprises SEQ ID NO: 53. In certain embodiments, the first polypeptide comprises SEQ ID NO: 49, the second polypeptide comprises SEQ ID NO: 50, and the third polypeptide comprises SEQ ID NO: 54. In certain embodiments, the first polypeptide comprises SEQ ID NO: 49, the second polypeptide comprises SEQ ID NO: 50, and the third polypeptide comprises SEQ ID NO: 69. In certain embodiments, the first polypeptide comprises SEQ ID NO: 49, the second polypeptide comprises SEQ ID NO: 50, and the third polypeptide comprises SEQ ID NO: 70. In certain embodiments, the first polypeptide comprises SEQ ID NO: 49, the second polypeptide comprises SEQ ID NO: 50, and the third polypeptide comprises SEQ ID NO: 71. In certain embodiments, the first polypeptide comprises SEQ ID NO: 49, the second polypeptide comprises SEQ ID NO: 50, and the third polypeptide comprises SEQ ID NO: 72. In certain embodiments, the first polypeptide comprises SEQ ID NO: 49, the second polypeptide comprises SEQ ID NO: 50, and the third polypeptide comprises SEQ ID NO: 90. In certain embodiments, the first polypeptide comprises SEQ ID NO: 49, the second polypeptide comprises SEQ ID NO: 50, and the third polypeptide comprises SEQ ID NO: 91. In certain embodiments, the first polypeptide comprises SEQ ID NO: 49, the second polypeptide comprises SEQ ID NO: 50, and the third polypeptide comprises SEQ ID NO: 92. In certain embodiments, the first polypeptide comprises SEQ ID NO: 49, the second polypeptide comprises SEQ ID NO: 50, and the third polypeptide comprises SEQ ID NO: 93. In certain embodiments, the first polypeptide comprises SEQ ID NO: 49, the second polypeptide comprises SEQ ID NO: 50, and the third polypeptide comprises SEQ ID NO: 94. In certain embodiments, the first polypeptide comprises SEQ ID NO: 49, the second polypeptide comprises SEQ ID NO: 50, and the third polypeptide comprises SEQ ID NO: 95. In certain embodiments, the first polypeptide comprises SEQ ID NO: 49, the second polypeptide comprises SEQ ID NO: 50, and the third polypeptide comprises SEQ ID NO: 96. In certain embodiments, the first polypeptide comprises SEQ ID NO: 49, the second polypeptide comprises SEQ ID NO: 50, and the third polypeptide comprises SEQ ID NO: 108. In certain embodiments, the first polypeptide comprises SEQ ID NO: 49, the second polypeptide comprises SEQ ID NO: 50, and the third polypeptide comprises SEQ ID NO: 111. In certain embodiments, the first polypeptide comprises SEQ ID NO: 49, the second polypeptide comprises SEQ ID NO: 50, and the third polypeptide comprises SEQ ID NO: 112. In certain embodiments, the first polypeptide comprises SEQ ID NO: 49, the second polypeptide comprises SEQ ID NO: 50, and the third polypeptide comprises SEQ ID NO: 113. In certain embodiments, the first polypeptide comprises SEQ ID NO: 49, the second polypeptide comprises SEQ ID NO: 50, and the third polypeptide comprises SEQ ID NO: 114. In certain embodiments, the first polypeptide comprises SEQ ID NO: 49, the second polypeptide comprises SEQ ID NO: 50, and the third polypeptide comprises SEQ ID NO: 115. In certain embodiments, the first polypeptide comprises SEQ ID NO: 49, the second polypeptide comprises SEQ ID NO: 50, and the third polypeptide comprises SEQ ID NO: 116. In certain embodiments, the first polypeptide comprises SEQ ID NO: 49, the second polypeptide comprises SEQ ID NO: 50, and the third polypeptide comprises SEQ ID NO: 117. In certain embodiments, the first polypeptide comprises SEQ ID NO: 49, the second polypeptide comprises SEQ ID NO: 50, and the third polypeptide comprises SEQ ID NO: 118. In certain embodiments, the first polypeptide comprises SEQ ID NO: 49, the second polypeptide comprises SEQ ID NO: 50, and the third polypeptide comprises SEQ ID NO: 119. In certain embodiments, the first polypeptide comprises SEQ ID NO: 49, the second polypeptide comprises SEQ ID NO: 50, and the third polypeptide comprises SEQ ID NO: 120. In certain embodiments, the first polypeptide comprises SEQ ID NO: 49, the second polypeptide comprises SEQ ID NO: 50, and the third polypeptide comprises SEQ ID NO: 121. In certain embodiments, the first polypeptide comprises SEQ ID NO: 49, the second polypeptide comprises SEQ ID NO: 50, and the third polypeptide comprises SEQ ID NO: 122. In certain embodiments, the first polypeptide comprises SEQ ID NO: 49, the second polypeptide comprises SEQ ID NO: 50, and the third polypeptide comprises SEQ ID NO: 123. In certain embodiments, the first polypeptide comprises SEQ ID NO: 49, the second polypeptide comprises SEQ ID NO: 50, and the third polypeptide comprises SEQ ID NO: 124. In certain embodiments, the first polypeptide comprises SEQ ID NO: 49, the second polypeptide comprises SEQ ID NO: 50, and the third polypeptide comprises SEQ ID NO: 125.

In certain embodiments, the antibody conjugate comprises a first polypeptide comprising a first sialidase, a first immunoglobulin Fc domain, and a first single chain variable fragment (scFv) (it is also understood that the scFv may be replaced by a first polypeptide chain of an immunoglobulin antigen binding fragment, e.g., Fab fragment); and a second polypeptide comprising a second sialidase, a second immunoglobulin Fc domain, and an optional second single chain variable fragment (scFv) (it is also understood that the scFv may be replaced by a second polypeptide chain of an immunoglobulin antigen binding fragment, e.g., Fab fragment). An example of this embodiment is shown in FIG. 1C. The first and second polypeptides can be covalently linked together. The covalent linkages can be disulfide bonds. In certain embodiments, the first scFv defines a first antigen-binding site, and the second scFv, when present, defines a second antigen-binding site. In certain embodiments, the first polypeptide comprises the first sialidase, the first immunoglobulin Fc domain, and the first scFv in an N- to C-terminal orientation. In certain embodiments, the first polypeptide comprises the first scFv, the first immunoglobulin Fc domain, and the first sialidase in an N- to C-terminal orientation. In certain embodiments, the second polypeptide comprises the second sialidase, the second immunoglobulin Fc domain, and the optional second scFv in an N- to C-terminal orientation. In certain embodiments, the second polypeptide comprises the second scFv, the second immunoglobulin Fc domain, and the second sialidase in an N- to C-terminal orientation.

In certain embodiments, the first polypeptide comprises the amino acid sequence of SEQ ID NO: 43, SEQ ID NO: 44, SEQ ID NO: 45, SEQ ID NO: 46, SEQ ID NO: 47, SEQ ID NO: 48, SEQ ID NO: 67, SEQ ID NO: 68, SEQ ID NO: 99, SEQ ID NO: 100, SEQ ID NO: 101, SEQ ID NO: 102, SEQ ID NO: 103, SEQ ID NO: 104, SEQ ID NO: 105, or SEQ ID NO: 110, or an amino acid sequence that has at least 85%, 90%, 95%, 96%, 97%, 98%, or 99% sequence identity to SEQ ID NO: 43, SEQ ID NO: 44, SEQ ID NO: 45, SEQ ID NO: 46, SEQ ID NO: 47, SEQ ID NO: 48, SEQ ID NO: 67, SEQ ID NO: 68, SEQ ID NO: 99, SEQ ID NO: 100, SEQ ID NO: 101, SEQ ID NO: 102, SEQ ID NO: 103, SEQ ID NO: 104, SEQ ID NO: 105, or SEQ ID NO: 110. In certain embodiments, the second polypeptide comprises the amino acid sequence of SEQ ID NO: 43, SEQ ID NO: 44, SEQ ID NO: 45, SEQ ID NO: 46, SEQ ID NO: 47, SEQ ID NO: 48, SEQ ID NO: 67, SEQ ID NO: 68, SEQ ID NO: 99, SEQ ID NO: 100, SEQ ID NO: 101, SEQ ID NO: 102, SEQ ID NO: 103, SEQ ID NO: 104, SEQ ID NO: 105, or SEQ ID NO: 110, or an amino acid sequence that has at least 85%, 90%, 95%, 96%, 97%, 98%, or 99% sequence identity to SEQ ID NO: 43, SEQ ID NO: 44, SEQ ID NO: 45, SEQ ID NO: 46, SEQ ID NO: 47, SEQ ID NO: 48, SEQ ID NO: 67, SEQ ID NO: 68, SEQ ID NO: 99, SEQ ID NO: 100, SEQ ID NO: 101, SEQ ID NO: 102, SEQ ID NO: 103, SEQ ID NO: 104, SEQ ID NO: 105, or SEQ ID NO: 110.

In certain embodiments, the first and/or second polypeptide comprises the amino acid sequence of

X₁X₂SX₃PX₄LQKESVFQSGAHAYRIPALLYLPGQQSLLAFAEQRASKKDE
HAELIVLRRGDYDAPTHQVQWQAQEVVAQARLDGHRSMNPCPLYDAQTGT
LFLFFIAIPGQVTEQQQLQTRANVTRLX₅QVTSTDHGRTWSSPRDLTDAA
IGPAYREWSTFAVGPGHCLQLHDRARSLVVPAYAYRKLHPX₆QRPIPSAF
X₇FLSHDHGRTWARGHFVAQDTLECQVAEVETGEQRVVTLNARSHLRARV
QAQSTNDGLDFQX₈SQLVKKLVEPPPQGX₉QGSVISFPSPRSGPGSPAQWL
LYTHPTHSWQRADLGAYLNPRPPAPEAWSEPX₁₀LLAKGSX₁₁AYSDLQSM
GTGPDGSPLFGX₁₂LYEANDYEEIVFLMFTLKQAFPAEYLPQGGGGSGGGG
SDKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKEY
KCKVSNKALPAPIEKTISKAKGQPREPQVYTLPPSREEMTKNQVSLTCLV
KGFYPSDIAVEWESNGQPENNYKTTPPVLDSDGSFFLYSKLTVDKSRWQQ
GNVFSCSVMHEALHNHYTQKSLSLSPGKGGGGSGGGGSGGGGSEVQLVES
GGGLVQPGGSLRLSCAASGFNIKDTYIHWVRQAPGKGLEWVARIYPTNGY
TRYADSVKGRFTISADTSKNTAYLQMNSLRAEDTAVYYCSRWGGDGFYAM
DYWGQGTLVTVSSGGGGSGGGGSGGGGSDIQMTQSPSSLSASVGDRVTIT
CRASQDVNTAVAWYQQKPGKAPKLLIYSASFLYSGVPSRFSGSRSGTDFT
LTISSLQPEDFATYYCQQHYTTPPTFGQGTKVEIK

(SEQ ID NO: 78), wherein X₁ is Ala, Arg, Asn, Asp, Gln, Glu, Gly, His, Leu, Lys, Met, Phe, Thr, Val, or not present, X₂ is Ala or Lys, X₃ is Asn or Leu, X₄ is Phe, Trp, Tyr or Val, X₅ is Ala, Cys, Ile, Ser, or Val, X₆ is Arg, Ile, or Lys, X₇ is Ala, Cys, Leu, or Val, X₈ is Glu or Lys, X₉ is Cys or Val, X₁₀ is Lys or Val, X₁₁ is Ala, Cys, Ser, or Val, and X₁₂ is Cys, Leu, or Val.

In certain embodiments, the first and/or second polypeptide comprises the amino acid sequence of

X₁ASLPX₂LQKESVFQSGAHAYRIPALLYLPGQQSLLAFAEQRASKKDEH
AELIVLRRGDYDAPTHQVQWQAQEVVAQARLDGHRSMNPCPLYDAQTGT
LFLFFIAIPGQVTEQQQLQTRANVTRLCQVTSTDHGRTWSSPRDLTDAA
IGPAYREWSTFAVGPGHCLQLHDRARSLVVPAYAYRKLHPX₃QRPIPSA
FCFLSHDHGRTWARGHFVAQDTLECQVAEVETGEQRVVTLNARSHLRAR
VQAQSTNDGLDFQESQLVKKLVEPPPQGCQGSVISFPSPRSGPGSPAQW
LLYTHPTHSWQRADLGAYLNPRPPAPEAWSEPVLLAKGSX₄AYSDLQSM
GTGPDGSPLFGCLYEANDYEEIVFLMFTLKQAFPAEYLPQGGGGSGGGG
SDKTHTCPPCPAPELLGGPSVFLFPPKPKDTLMISRTPEVTCVVVDVSH
EDPEVKFNWYVDGVEVHNAKTKPREEQYNSTYRVVSVLTVLHQDWLNGK
EYKCKVSNKALPAPIEKTISKAKGQPREPQVYTLPPSREEMTKNQVSLT
CLVKGFYPSDIAVEWESNGQPENNYKTTPPVLDSDGSFFLYSKLTVDKS
RWQQGNVFSCSVMHEALHNHYTQKSLSLSPGKGGGGSGGGGSGGGGSEV
QLVESGGGLVQPGGSLRLSCAASGFNIKDTYIHWVRQAPGKGLEWVARI
YPTNGYTRYADSVKGRFTISADTSKNTAYLQMNSLRAEDTAVYYCSRWG
GDGFYAMDYWGQGTLVTVSSGGGGSGGGGSGGGGSDIQMTQSPSSLSAS
VGDRVTITCRASQDVNTAVAWYQQKPGKAPKLLIYSASFLYSGVPSRFS
GSRSGTDFTLTISSLQPEDFATYYCQQHYTTPPTFGQGTKVEIK

(SEQ ID NO: 75), wherein X₁ is Ala, Asn, Asp, His, Leu, Met, Phe, Thr, or not present, X₂ is Phe, Trp, Tyr or Val, X₃ is Arg, Ile, or Lys, and X₄ is Ala, Cys, Ser, or Val. In certain embodiments, X₁ is Ala, Asp, Met, or not present, X₂ is Tyr or Val, X₃ is Ile or Lys, and X₄ is Ala or Cys.

In certain embodiments, the first and/or second polypeptide comprises the amino acid sequence of

X₁X₂SX₃X₄X₅LQXESVFQSGAHAYRIPALLYLPGQQSLLAFAEQRASX₇X₈
DEHAELIVX₉RRGDYDAX₁₀THQVQWX₁₁AQEVVAQAX₁₂LDGHRSMNPCPL
YDX₁₃QTGTLFLFFIAIPX₁₄X₁₅VTEX₁₆QQLQTRANVTRLX₁₇X₁₈VTSTDH
GRTWSSPRDLTDAAIGPX₁₉YREWSTFAVGPGHX₂₀LQLHDRX₂₁RSLVVPA
YAYRKLHPX₂₂QRPIPSAFX₂₃FLSHDHGRTWARGHFVAQDTX₂₄ECQVAEV
ETGEQRVVTLNARSHLRARVQAQSX₂₅NX₂₆GLDFQX₂₇SQLVKKLVEPPP
X₂₈GX₂₉QGSVISFPSPRSGPGSPAQX₃₀LLYTHPTHX₃₁X₃₂QRADLGAYLN
PRPPAPEAWSEPX₃₃LLAKGSX₃₄AYSDLQSMGTGPDGSPLFGX₃₅LYEAND
YEEIX₃₆FX₃₇MFTLKQAFPAEYLPQGGGGSGGGGSDKTHTCPPCPAPELL
GGPSVFLFPPKPKDTLMISRTPEVTCVVVDVSHEDPEVKFNWYVDGVEV
HNAKTKPREEQYNSTYRVVSVLTVLHQDWLNGKEYKCKVSNKALPAPIE
KTISKAKGQPREPQVYTLPPSREEMTKNQVSLTCLVKGFYPSDIAVEWE
SNGQPENNYKTTPPVLDSDGSFFLYSKLTVDKSRWQQGNVFSCSVMHEA
LHNHYTQKSLSLSPGKGGGGSGGGGSGGGGSEVQLVESGGGLVQPGGSL
RLSCAASGFNIKDTYIHWVRQAPGKGLEWVARIYPTNGYTRYADSVKGR
FTISADTSKNTAYLQMNSLRAEDTAVYYCSRWGGDGFYAMDYWGQGTLV
TVSSGGGGSGGGGSGGGGSDIQMTQSPSSLSASVGDRVTITCRASQDVN
TAVAWYQQKPGKAPKLLIYSASFLYSGVPSRFSGSRSGTDFTLTISSLQ
PEDFATYYCQQHYTTPPTFGQGTKVEIK

(SEQ ID NO: 107), wherein X₁ is Ala, Arg, Asn, Asp, Gln, Glu, Gly, His, Leu, Lys, Met, Phe, Thr, Val, or not present, X₂ is Ala or Lys, X₃ is Asn or Leu, X₄ is Pro or His, X₅ is Phe, Trp, Tyr or Val, X₆ is Lys or Asp. X₇ is Lys, Arg, or Glu. X₈ is Lys, Ala, Arg, or Glu, X₉ is Leu or Met, X₁₀ is Pro, Asn, Asp, His, Glu, Gly, Ser or Thr, X₁₁ is Gln or His, X₁₂ is Arg or Lys, X₁₃ is Ala, Glu or Lys, X₁₄ is Gly or Asp, X₁₅ is Gln or His, X₁₆ is Gln, Arg, or Lys, X₁₇ is Ala, Cys, Ile, Ser, Val, or Leu, X₁₈ is Gln or Leu, X₁₉ is Ala or Val, X₂₀ is Cys or Gly, X₂₁ is Ala or Gly, X₂₂ is Arg, Ile, or Lys, X₂₃ is Ala, Cys, Leu, or Val, X₂₄ is Leu, Ala, or Val, X₂₅ is Thr or Ala, X₂₆ is Asp or Gly, X₂₇ is Glu or Lys, X₂₈ is Gln, Ala, His, Phe, or Pro, X₂₉ is Cys or Val, X₃₀ is Trp or Arg, X₃₁ is Ser or Arg, X₃₂ is Trp or Lys, X₃₃ is Lys or Val, X₃₄ is Ala, Cys, Ser, or Val, X₃₅ is Cys, Leu, or Val, X₃₅ is Val or Arg, and X₃₇ is Leu, Gln, His, Ile, Lys, or Ser, and the sialidase comprises at least one mutation relative to wild-type human Neu2 (SEQ ID NO: 2).

In certain embodiments, the first and/or second polypeptide comprises the amino acid sequence of

X₁ASLPX₂LQX₃ESVFQSGAHAYRIPALLYLPGQQSLLAFAEQRASKKDE
HAELIVLRRGDYDAX₄THQVQWQAQEVVAQARLDGHRSMNPCPLYDX₅QT
GTLFLFFIAIPGQVTEQQQLQTRANVTRLCQVTSTDHGRTWSSPRDLTD
AAIGPAYREWSTFAVGPGHCLQLHDRARSLVVPAYAYRKLHPX₆QRPIP
SAFCFLSHDHGRTWARGHEVAQDTLECQVAEVETGEQRVVTLNARSHLR
ARVQAQSTNDGLDFQESQLVKKLVEPPPX₇GCQGSVISFPSPRSGPGSP
AQWLLYTHPTHX₈X₉QRADLGAYLNPRPPAPEAWSEPVLLAKGSX₁₀AYS
DLQSMGTGPDGSPLFGCLYEANDYEEIX₁₁FX₁₂MFTLKQAFPAEYLPQG
GGGSGGGGSDKTHTCPPCPAPELLGGPSVFLFPPKPKDTLMISRTPEVT
CVVVDVSHEDPEVKFNWYVDGVEVHNAKTKPREEQYNSTYRVVSVLTVL
HQDWLNGKEYKCKVSNKALPAPIEKTISKAKGQPREPQVYTLPPSREEM
TKNQVSLTCLVKGFYPSDIAVEWESNGQPENNYKTTPPVLDSDGSFFLY
SKLTVDKSRWQQGNVFSCSVMHEALHNHYTQKSLSLSPGKGGGGSGGGG
SGGGGSEVQLVESGGGLVQPGGSLRLSCAASGFNIKDTYIHWVRQAPGK
GLEWVARIYPTNGYTRYADSVKGRFTISADTSKNTAYLQMNSLRAEDTA
VYYCSRWGGDGFYAMDYWGQGTLVTVSSGGGGSGGGGSGGGGSDIQMTQ
SPSSLSASVGDRVTITCRASQDVNTAVAWYQQKPGKAPKLLIYSASFLY
SGVPSRFSGSRSGTDFTLTISSLQPEDFATYYCQQHYTTPPTFGQGTKV
EIK

(SEQ ID NO: 106), wherein X₁ is Ala, Arg, Asn, Asp, Gln, Glu, Gly, His, Leu, Lys, Met, Phe, Thr, Val, or not present, X₂ is Phe, Trp, Tyr or Val, X₃ is Lys or Asp, X₄ is Pro, Asn, Asp, His, Glu, Gly, Ser or Thr, X₅ is Ala, Glu, or Lys, X₆ is Arg, Ile, or Lys, X₇ is Gln, Ala, His, Phe, or Pro, X₈ is Ser or Arg, X₉ is Trp or Lys, X₁₀ is Ala, Cys, Ser, or Val, X₁₁ is Val or Arg, and X₁₂ is Leu, Gln, His, Ile, Lys, or Ser, and the sialidase comprises at least one mutation relative to wild-type human Neu2 (SEQ ID NO: 2). In certain embodiments, X₁ is Ala, Asp, Met, or not present, X₂ is Tyr or Val, X₃ is Lys or Asp, X₄ is Pro, Asn, Gly, Ser or Thr, X₅ is Ala or Glu, X₆ is Ile or Lys, X₇ is Gln or Ala, X₈ is Ser or Arg, X₉ is Trp or Lys, X₁₀ is Ala or Cys, X₁₁ is Val or Arg, and X₁₂ is Leu or Ile.

In certain embodiments, the first and second polypeptide comprise SEQ ID NO: 43. In certain embodiments, the first and second polypeptide comprise SEQ ID NO: 44. In certain embodiments, the first and second polypeptide comprise SEQ ID NO: 45. In certain embodiments, the first and second polypeptide comprise SEQ ID NO: 46. In certain embodiments, the first and second polypeptide comprise SEQ ID NO: 47. In certain embodiments, the first and second polypeptide comprise SEQ ID NO: 48. In certain embodiments, the first and second polypeptide comprise SEQ ID NO: 67. In certain embodiments, the first and second polypeptide comprise SEQ ID NO: 68. In certain embodiments, the first and second polypeptide comprise SEQ ID NO: 99. In certain embodiments, the first and second polypeptide comprise SEQ ID NO: 100. In certain embodiments, the first and second polypeptide comprise SEQ ID NO: 101. In certain embodiments, the first and second polypeptide comprise SEQ ID NO: 102. In certain embodiments, the first and second polypeptide comprise SEQ ID NO: 103. In certain embodiments, the first and second polypeptide comprise SEQ ID NO: 104. In certain embodiments, the first and second polypeptide comprise SEQ ID NO: 105. In certain embodiments, the first and second polypeptide comprise SEQ ID NO: 110.

In certain embodiments, the antibody conjugate has a molecular weight from about 135 kDa to about 165 kDa, e.g., about 140 kDa. In other embodiments, the antibody conjugate has a molecular weight from about 215 kDa to about 245 kDa, e.g., about 230 kDa.

In certain embodiments, the antibody conjugate comprises two polypeptides that each comprise an immunoglobulin Fc domain, and the first polypeptide has either a “knob” mutation, e.g., T366Y, or a “hole” mutation, e.g., Y407T, for heterodimerization with the second polypeptide, and the second polypeptide has either a respective “knob” mutation, e.g., T366Y, or a “hole” mutation, e.g., Y407T, for heterodimerization with the first polypeptide (residue numbers according to EU numbering, Kabat, E. A., et al. (1991) supra). For example, in certain embodiments, the antibody comprises two polypeptides that each comprise an immunoglobulin Fc domain derived from human IgG1 Fc domain, and the first polypeptide comprises a Y407T mutation (e.g., the first polypeptide comprises SEQ ID NO: 30), and the second polypeptide comprises a T366Y mutation (e.g., the second polypeptide comprises SEQ ID NO: 31).

As used herein, the term “multispecific antibody” is understood to mean an antibody that specifically binds to at least two different antigens, i.e., an antibody that comprises at least two antigen-binding sites that bind to at least two different antigens. As used herein, the term “bispecific antibody” is understood to mean an antibody that specifically binds to two different antigens, i.e., an antibody that comprises two antigen-binding sites each of which bind to separate and distinct antigens. In other words, a first binding site binds a first antigen and a second binding site binds a second, different antigen. A multispecific or bispecific antibody may, for example, be a human or humanized antibody, and/or be a full length antibody or an antibody fragment (e.g., a F(ab′)₂ bispecific antibody).

The present disclosure encompasses antibody conjugates comprising antibody fragments, which may be generated by traditional means, such as enzymatic digestion, or by recombinant techniques. For a review of certain antibody fragments, see Hudson et al. (2003) supra.

In certain embodiments, the antibody conjugate or fusion protein can be covalently or non-covalently associated with a biological modifier, wherein the biological modifier can be used to enhance the solubility of the antibody, increase binding specificity, decrease immunogenicity or toxicity or modify the pharmacokinetic profile of the antibody. For example, the biological modifier can be used to increase the molecular weight of the antibody to increase its circulating half-life.

It is contemplated that the antibody conjugate or fusion protein may be covalently bound to one or more (for example, 2, 3, 4, 5, 6, 8, 9, 10 or more) biological modifiers that may comprise linear or branched polymers. Exemplary biological modifiers may include, for example, a variety of polymers, such as those described in U.S. Pat. No. 7,842,789. Particularly useful are polyalkylene ethers such as polyethylene glycol (PEG) and derivatives thereof (for example, alkoxy polyethylene glycol, for example, methoxypolyethylene glycol, ethoxypolyethylene glycol and the like); block copolymers of polyoxyethylene and polyoxypropylene (Pluronics); polymethacrylates; carbomers; and branched or unbranched polysaccharides which comprise the saccharide monomers such as D-mannose, D- and L-galactose, fucose, fructose, D-xylose, L-arabinose, and D-glucuronic acid.

In other embodiments, the biological modifier can be a hydrophilic polyvinyl polymer such as polyvinyl alcohol and polyvinylpyrrolidone (PVP)-type polymers. The biological modifier can be a functionalized polyvinylpyrrolidone, for example, carboxy or amine functionalized on one (or both) ends of the polymer (as available from PolymerSource). Alternatively, the biological modifier can include Poly N-(2-hydroxypropyl)methacrylamide (HPMA), or functionalized HPMA (amine, carboxy, etc.), Poly(N-isopropylacrylamide) or functionalized poly(N-isopropylacrylamide). Alternatively, the biological modifier can include Poly N-(2-hydroxypropyl)methacrylamide (HPMA), or functionalized HPMA (amine, carboxy, etc.), Poly(N-isopropylacrylamide) or functionalized poly(N-isopropylacrylamide). The modifier prior to conjugation need not be, but preferably is, water soluble, but the final conjugate should be water soluble.

In general, the biological modifier may have a molecular weight from about 2 kDa to about 5 kDa, from about 2 kDa to about 10 kDa, from about 2 kDa to about 20 kDa, from about 2 kDa to about 30 kDa, from about 2 kDa to about 40 kDa, from about 2 kDa to about 50 kDa, from about 2 kDa to about 60 kDa, from about 2 kDa to about 70 kDa, from about 2 kDa to about 80 kDa, from about 2 kDa to about 90 kDa, from about 2 kDa to about 100 kDa, from about 2 kDa to about 150 kDa, from about 5 kDa to about 10 kDa, from about 5 kDa to about 20 kDa, from about 5 kDa to about 30 kDa, from about 5 kDa to about 40 kDa, from about 5 kDa to about 50 kDa, from about 5 kDa to about 60 kDa, from about 5 kDa to about 70 kDa, from about 5 kDa to about 80 kDa, from about 5 kDa to about 90 kDa, from about 5 kDa to about 100 kDa, from about 5 kDa to about 150 kDa, from about 10 kDa to about 20 kDa, from about 10 kDa to about 30 kDa, from about 10 kDa to about 40 kDa, from about 10 kDa to about 50 kDa, from about 10 kDa to about 60 kDa, from about 10 kDa to about 70 kDa, from about 10 kDa to about 80 kDa, from about 10 kDa to about 90 kDa, from about 10 kDa to about 100 kDa, from about 10 kDa to about 150 kDa, from about 20 kDa to about 30 kDa, from about 20 kDa to about 40 kDa, from about 20 kDa to about 50 kDa, from about 20 kDa to about 60 kDa, from about 20 kDa to about 70 kDa, from about 20 kDa to about 80 kDa, from about 20 kDa to about 90 kDa, from about 20 kDa to about 100 kDa, from about 20 kDa to about 150 kDa, from about 30 kDa to about 40 kDa, from about 30 kDa to about 50 kDa, from about 30 kDa to about 60 kDa, from about 30 kDa to about 70 kDa, from about 30 kDa to about 80 kDa, from about 30 kDa to about 90 kDa, from about 30 kDa to about 100 kDa, from about 30 kDa to about 150 kDa, from about 40 kDa to about 50 kDa, from about 40 kDa to about 60 kDa, from about 40 kDa to about 70 kDa, from about 40 kDa to about 80 kDa, from about 40 kDa to about 90 kDa, from about 40 kDa to about 100 kDa, from about 40 kDa to about 150 kDa, from about 50 kDa to about 60 kDa, from about 50 kDa to about 70 kDa, from about 50 kDa to about 80 kDa, from about 50 kDa to about 90 kDa, from about 50 kDa to about 100 kDa, from about 50 kDa to about 150 kDa, from about 60 kDa to about 70 kDa, from about 60 kDa to about 80 kDa, from about 60 kDa to about 90 kDa, from about 60 kDa to about 100 kDa, from about 60 kDa to about 150 kDa, from about 70 kDa to about 80 kDa, from about 70 kDa to about 90 kDa, from about 70 kDa to about 100 kDa, from about 70 kDa to about 150 kDa, from about 80 kDa to about 90 kDa, from about 80 kDa to about 100 kDa, from about 80 kDa to about 150 kDa, from about 90 kDa to about 100 kDa, from about 90 kDa to about 150 kDa, or from about 100 kDa to about 150 kDa.

It is contemplated that the antibody conjugate or fusion protein is attached to about 10 or fewer polymer molecules (e.g., 9, 8, 7, 6, 5, 4, 3, 2, or 1), each polymer molecule having a molecular weight of at least about 20,000 D, or at least about 30,000 D, or at least about 40,000 D.

Although a variety of polymers can be used as biological modifiers, it is contemplated that the antibody conjugates or fusion proteins described herein may be attached to polyethylene glycol (PEG) polymers. In one embodiment, the antibody conjugate or fusion protein described herein is covalently attached to at least one PEG having an actual MW of at least about 20,000 D. In another embodiment, the antibody conjugate or fusion protein described herein is covalently attached to at least one PEG having an actual MW of at least about 30,000 D. In another embodiment, the antibody conjugate or fusion protein described herein is covalently attached to at least one PEG having an actual MW of at least about 40,000 D. In certain embodiments, the PEG is methoxyPEG(5000)-succinimidylpropionate (mPEG-SPA), methoxyPEG(5000)-succinimidylsuccinate (mPEG-SS). Such PEGS are commercially available from Nektar Therapeutics or SunBiowest.

Attachment sites on an antibody conjugate or fusion protein for a biological modifier include the N-terminal amino group and epsilon amino groups found on lysine residues, as well as other amino, imino, carboxyl, sulfhydryl, hydroxyl or other hydrophilic groups. The polymer may be covalently bonded directly to the antibody conjugate or fusion protein with or without the known use of a multifunctional (ordinarily bifunctional) crosslinking agent using chemistries and used in the art. For example, sulfhydryl groups can be derivatized by coupling to maleimido-substituted PEG (e.g. alkoxy-PEG amine plus sulfosuccinimidyl 4-(N-maleimidomethyl)cyclohexane-1-carboxylate), or PEG-maleimide commercially available from Shearwater Polymers, Inc., Huntsville, Ala.).

Additional exemplary sialidase fusion proteins and antibody sialidase conjugates are described in U.S. Provisional Patent Application No. 62/870,354, filed Jul. 3, 2019 (including in the Detailed Description in the section entitled “I. Sialidase anti-PD-L1 Fusion Proteins,” and in the Examples in Examples 3 and 4), U.S. Provisional Patent Application No. 62/870,348, filed Jul. 3, 2019 (including in the Detailed Description in the section entitled “I. Sialidase anti-CD20 Fusion Proteins,” and in the Examples in Examples 3, 4, 5, 6, and 7), U.S. Provisional Patent Application No. 62/870,347, filed Jul. 3, 2019 (including in the Detailed Description in the section entitled “I. Sialidase anti-EGFR Fusion Proteins,” and in the Examples in Examples 3, 4, and 5), and U.S. Provisional Patent Application No. 62/870,341, filed Jul. 3, 2019 (including in the Detailed Description in the section entitled “I. Sialidase anti-HER2 Fusion Proteins,” and in the Examples in Examples 3, 4, 5, 6, 7, and 8).

### Methods of Making a Sialidase, Fusion Protein, or Antibody Conjugate

Methods for producing sialidases, fusion proteins, e.g., those disclosed herein, antibodies, or antibody conjugates, e.g., those disclosed herein, are known in the art. For example, DNA molecules encoding light chain variable regions and/or heavy chain variable regions can be synthesized chemically or by recombinant DNA methodologies. For example, the sequences of the antibodies can be cloned from hybridomas by conventional hybridization techniques or polymerase chain reaction (PCR) techniques, using the appropriate synthetic nucleic acid primers. The resulting DNA molecules encoding the variable regions of interest can be ligated to other appropriate nucleotide sequences, including, for example, constant region coding sequences, and expression control sequences, to produce conventional gene expression constructs (i.e., expression vectors) encoding the desired antibodies. Production of defined gene constructs is within routine skill in the art.

Nucleic acids encoding desired sialidases, fusion proteins, and/or antibody conjugates can be incorporated (ligated) into expression vectors, which can be introduced into host cells through conventional transfection or transformation techniques. Exemplary host cells are E. coli cells, Chinese hamster ovary (CHO) cells, human embryonic kidney 293 (HEK 293) cells, HeLa cells, baby hamster kidney (BHK) cells, monkey kidney cells (COS), human hepatocellular carcinoma cells (e.g., Hep G2), and myeloma cells that do not otherwise produce IgG protein. Transformed host cells can be grown under conditions that permit the host cells to express the genes that encode the immunoglobulin light and/or heavy chain variable regions.

Specific expression and purification conditions will vary depending upon the expression system employed. For example, if a gene is to be expressed in E. coli, it is first cloned into an expression vector by positioning the engineered gene downstream from a suitable bacterial promoter, e.g., Trp or Tac, and a prokaryotic signal sequence. The expressed protein may be secreted. The expressed protein may accumulate in refractile or inclusion bodies, which can be harvested after disruption of the cells by French press or sonication. The refractile bodies then are solubilized, and the protein may be refolded and/or cleaved by methods known in the art.

If the engineered gene is to be expressed in eukaryotic host cells, e.g., CHO cells, it is first inserted into an expression vector containing a suitable eukaryotic promoter, a secretion signal, a poly A sequence, and a stop codon. Optionally, the vector or gene construct may contain enhancers and introns. In embodiments involving fusion proteins comprising an antibody or portion thereof, the expression vector optionally contains sequences encoding all or part of a constant region, enabling an entire, or a part of, a heavy or light chain to be expressed. The gene construct can be introduced into eukaryotic host cells using conventional techniques.

The host cells express a sialidase or a fusion protein and/or antibody conjugate comprising a sialidase and VL or VH fragments, VL-VH heterodimers, VH-VL or VL-VH single chain polypeptides, complete heavy or light immunoglobulin chains, or portions thereof, each of which may be attached to a moiety having another function (e.g., cytotoxicity). In some embodiments involving fusion proteins and/or antibody conjugates, a host cell is transfected with a single vector expressing a polypeptide expressing a sialidase and an entire, or part of, a heavy chain (e.g., a heavy chain variable region) or a sialidase and a light chain (e.g., a light chain variable region), or a polypeptide expressing an entire, or part of, a heavy chain (e.g., a heavy chain variable region) or a light chain (e.g., a light chain variable region). In some embodiments, a host cell is transfected with a single vector encoding (a) a polypeptide comprising a heavy chain variable region and a polypeptide comprising a light chain variable region, or (b) an entire immunoglobulin heavy chain and an entire immunoglobulin light chain, wherein in (a) or in (b), the polypeptide may also comprise a sialidase. In some embodiments, a host cell is co-transfected with more than one expression vector (e.g., one expression vector expressing a polypeptide comprising an entire, or part of, a heavy chain or heavy chain variable region, optionally comprising a sialidase fused thereto, and another expression vector expressing a polypeptide comprising an entire, or part of, a light chain or light chain variable region, optionally comprising a sialidase fused thereto).

A polypeptide comprising a sialidase or a fusion protein, e.g., a fusion protein comprising an immunoglobulin heavy chain variable region or light chain variable region, can be produced by growing (culturing) a host cell transfected with an expression vector encoding such a variable region, under conditions that permit expression of the polypeptide. Following expression, the polypeptide can be harvested and purified or isolated using techniques known in the art, e.g., affinity tags such as glutathione-S-transferase (GST) or histidine tags.

In embodiments in which a fusion protein and/or antibody conjugate is produced, a sialidase fused to a monoclonal antibody, Fc domain, or an antigen-binding domain of the antibody, can be produced by growing (culturing) a host cell transfected with: (a) an expression vector that encodes a complete or partial immunoglobulin heavy chain, and a separate expression vector that encodes a complete or partial immunoglobulin light chain; or (b) a single expression vector that encodes both chains (e.g., complete or partial heavy and light chains), under conditions that permit expression of both chains. The sialidase will be fused to one or more of the chains. The intact fusion protein and/or antibody conjugate can be harvested and purified or isolated using techniques known in the art, e.g., Protein A, Protein G, affinity tags such as glutathione-S-transferase (GST) or histidine tags. It is within ordinary skill in the art to express the heavy chain and the light chain from a single expression vector or from two separate expression vectors.

In certain embodiments, in order to express a protein, e.g., a sialidase, as a secreted protein, a native N-terminal signal sequence of the protein is replaced, e.g., with MDMRVPAQLLGLLLLWLPGARC (SEQ ID NO: 32). In certain embodiments, to express a protein, e.g., a recombinant human sialidase, as a secreted protein, an N-terminal signal sequence, e.g., MDMRVPAQLLGLLLLWLPGARC (SEQ ID NO: 32), is added. Additional exemplary N-terminal signal sequences include signal sequences from interleukin-2, CD-5, IgG kappa light chain, trypsinogen, serum albumin, and prolactin. In certain embodiments, in order to express a protein, e.g., a sialidase, as a secreted protein, a C terminal lysosomal signal motif, e.g., YGTL (SEQ ID NO: 33) is removed.

Methods for reducing or eliminating the antigenicity of antibodies and antibody fragments are known in the art. When the antibodies are to be administered to a human, the antibodies preferably are “humanized” to reduce or eliminate antigenicity in humans. Preferably, each humanized antibody has the same or substantially the same affinity for the antigen as the non-humanized mouse antibody from which it was derived.

In one humanization approach, chimeric proteins are created in which mouse immunoglobulin constant regions are replaced with human immunoglobulin constant regions. See, e.g., Morrison et al., 1984, PROC. NAT. ACAD. SCI. 81:6851-6855, Neuberger et al., 1984, NATURE 312:604-608; U.S. Pat. No. 6,893,625 (Robinson); U.S. Pat. No. 5,500,362 (Robinson); and U.S. Pat. No. 4,816,567 (Cabilly).

In an approach known as CDR grafting, the CDRs of the light and heavy chain variable regions are grafted into frameworks from another species. For example, murine CDRs can be grafted into human FRs. In some embodiments, the CDRs of the light and heavy chain variable regions of an antibody are grafted into human FRs or consensus human FRs. To create consensus human FRs, FRs from several human heavy chain or light chain amino acid sequences are aligned to identify a consensus amino acid sequence. CDR grafting is described in U.S. Pat. No. 7,022,500 (Queen); U.S. Pat. No. 6,982,321 (Winter); U.S. Pat. No. 6,180,370 (Queen); U.S. Pat. No. 6,054,297 (Carter); U.S. Pat. No. 5,693,762 (Queen); U.S. Pat. No. 5,859,205 (Adair); U.S. Pat. No. 5,693,761 (Queen); U.S. Pat. No. 5,565,332 (Hoogenboom); U.S. Pat. No. 5,585,089 (Queen); U.S. Pat. No. 5,530,101 (Queen); Jones et al. (1986) NATURE 321: 522-525; Riechmann et al. (1988) NATURE 332: 323-327; Verhoeyen et al. (1988) SCIENCE 239: 1534-1536; and Winter (1998) FEBS LETT 430: 92-94.

In an approach called “SUPERHUMANIZATION™,” human CDR sequences are chosen from human germline genes, based on the structural similarity of the human CDRs to those of the mouse antibody to be humanized. See, e.g., U.S. Pat. No. 6,881,557 (Foote); and Tan et al., 2002, J. IMMUNOL. 169:1119-1125.

Other methods to reduce immunogenicity include “reshaping,” “hyperchimerization,” and “veneering/resurfacing.” See, e.g., Vaswami et al., 1998, ANNALS OF ALLERGY, ASTHMA, & IMMUNOL. 81:105; Roguska et al., 1996, PROT. ENGINEER 9:895-904; and U.S. Pat. No. 6,072,035 (Hardman). In the veneering/resurfacing approach, the surface accessible amino acid residues in the murine antibody are replaced by amino acid residues more frequently found at the same positions in a human antibody. This type of antibody resurfacing is described, e.g., in U.S. Pat. No. 5,639,641 (Pedersen).

Another approach for converting a mouse antibody into a form suitable for medical use in humans is known as ACTIVMAB™ technology (Vaccinex, Inc., Rochester, NY), which involves a vaccinia virus-based vector to express antibodies in mammalian cells. High levels of combinatorial diversity of IgG heavy and light chains can be produced. See, e.g., U.S. Pat. No. 6,706,477 (Zauderer); U.S. Pat. No. 6,800,442 (Zauderer); and U.S. Pat. No. 6,872,518 (Zauderer). Another approach for converting a mouse antibody into a form suitable for use in humans is technology practiced commercially by KaloBios Pharmaceuticals, Inc. (Palo Alto, CA). This technology involves the use of a proprietary human “acceptor” library to produce an “epitope focused” library for antibody selection. Another approach for modifying a mouse antibody into a form suitable for medical use in humans is HUMAN ENGINEERING™ technology, which is practiced commercially by XOMA (US) LLC. See, e.g., International (PCT) Publication No. WO 93/11794 and U.S. Pat. No. 5,766,886 (Studnicka); U.S. Pat. No. 5,770,196 (Studnicka); U.S. Pat. No. 5,821,123 (Studnicka); and U.S. Pat. No. 5,869,619 (Studnicka).

Any suitable approach, including any of the above approaches, can be used to reduce or eliminate human immunogenicity of an antibody.

In addition, it is possible to create fully human antibodies in mice. Fully human mAbs lacking any non-human sequences can be prepared from human immunoglobulin transgenic mice by techniques referenced in, e.g., Lonberg et al., NATURE 368:856-859, 1994; Fishwild et al., NATURE BIOTECHNOLOGY 14:845-851, 1996; and Mendez et al., NATURE GENETICS 15:146-156, 1997. Fully human monoclonal antibodies can also be prepared and optimized from phage display libraries by techniques referenced in, e.g., Knappik et al., J. MOL. BIOL. 296:57-86, 2000; and Krebs et al., J. IMMUNOL. METH. 254:67-84 2001).

The present invention encompasses fusion proteins comprising antibody fragments, which may be generated by traditional means, such as enzymatic digestion, or by recombinant techniques. For a review of certain antibody fragments, see Hudson et al. (2003) NAT. MED. 9:129-134.

Various techniques have been developed for the production of antibody fragments. Traditionally, these fragments were derived via proteolytic digestion of intact antibodies (see, e.g., Morimoto et al. (1992) Journal of Biochemical and Biophysical Methods 24:107-117; and Brennan et al. (1985) Science 229:81). However, these fragments can now be produced directly by recombinant host cells. Fab, Fv and ScFv antibody fragments can all be expressed in and secreted from E. coli, thus allowing the facile production of large amounts of these fragments. Antibody fragments can be isolated from the antibody phage libraries. Alternatively, Fab′-SH fragments can be directly recovered from E. coli and chemically coupled to form F(ab′)2 fragments (Carter et al. (1992) Bio/Technology 10:163-167). According to another approach, F(ab′)2 fragments can be isolated directly from recombinant host cell culture. Fab and F(ab′)2 fragments with increased in vivo half-life comprising salvage receptor binding epitope residues are described in U.S. Pat. No. 5,869,046. Other techniques for the production of antibody fragments will be apparent to the skilled practitioner. In certain embodiments, an antibody is a single chain Fv fragment (scFv). See U.S. Pat. Nos. 5,571,894 and 5,587,458.

Methods for making bispecific antibodies are known in the art. See Milstein and Cuello (1983) NATURE 305:537, International (PCT) Publication No. WO93/08829, and Traunecker et al. (1991) EMBO J., 10:3655. For further details of generating bispecific antibodies see, for example, Suresh et al. (1986) METHODS ENZYMOL. 121:210. Bispecific antibodies include cross-linked or “heteroconjugate” or “heterodimer” antibodies. For example, one of the antibodies in the heterodimer can be coupled to avidin, the other to biotin. Heterodimer antibodies may be made using any convenient cross-linking method. Suitable cross-linking agents are well known in the art, and are disclosed in U.S. Pat. No. 4,676,980, along with a number of cross-linking techniques.

Examples of heterodimeric or asymmetric IgG-like molecules include but are not limited to those obtained with the following technologies or using the following formats: Triomab/Quadroma, Knobs-into-Holes, CrossMabs, electrostatically-matched antibodies, LUZ-Y, Strand Exchange Engineered Domain body, Biclonic and DuoBody.

Advantages of using antibody fragments (e.g., F(ab) and F(ab′)2 fragments) include the elimination of non-specific binding between Fc portions of antibodies and Fc receptors on cells (such as macrophages, dendritic cells, neutrophils, NK cells and B cells). In addition, they may be able to penetrate tissues more efficiently due to their smaller size.

Heterodimeric antibodies, or asymmetric antibodies, allow for greater flexibility and new formats for attaching a variety of drugs to the antibody arms. One of the general formats for creating a heterodimeric antibody is the “knobs-into-holes” format. This format is specific to the heavy chain part of the constant region in antibodies. The “knobs” part is engineered by replacing a small amino acid with a larger one, which fits into a “hole”, which is engineered by replacing a large amino acid with a smaller one. What connects the “knobs” to the “holes” are the disulfide bonds between each chain. The “knobs-into-holes” shape facilitates antibody dependent cell mediated cytotoxicity. Single chain variable fragments (scFv) are connected to the variable domain of the heavy and light chain via a short linker peptide. The linker is rich in glycine, which gives it more flexibility, and serine/threonine, which gives it specificity. Two different scFv fragments can be connected together, via a hinge region, to the constant domain of the heavy chain or the constant domain of the light chain. This gives the antibody bispecificity, allowing for the binding specificities of two different antigens. The “knobs-into-holes” format enhances heterodimer formation but doesn't suppress homodimer formation.

Several approaches to support heterodimerization have been described, for example in International (PCT) Publication Nos. WO96/27011, WO98/050431, WO2007/110205, WO2007/147901, WO2009/089004, WO2010/129304, WO2011/90754, WO2011/143545, WO2012/058768, WO2013/157954, and WO2013/096291, and European Patent Publication No. EP1870459. Typically, in the approaches known in the art, the CH3 domain of the first heavy chain and the CH3 domain of the second heavy chain are both engineered in a complementary manner so that the heavy chain comprising one engineered CH3 domain can no longer homodimerize with another heavy chain of the same structure (e.g. a CH3-engineered first heavy chain can no longer homodimerize with another CH3-engineered first heavy chain; and a CH3-engineered second heavy chain can no longer homodimerize with another CH3-engineered second heavy chain). Thereby the heavy chain comprising one engineered CH3 domain is forced to heterodimerize with another heavy chain comprising the CH3 domain, which is engineered in a complementary manner. As a result, the CH3 domain of the first heavy chain and the CH3 domain of the second heavy chain are engineered in a complementary manner by amino acid substitutions, such that the first heavy chain and the second heavy chain are forced to heterodimerize, whereas the first heavy chain and the second heavy chain can no longer homodimerize (e.g., for steric reasons).

### III. Expression Methods

A protein of interest, e.g., a chimeric antigen receptor and/or a sialidase, may be expressed in a cell of interest by incorporating a gene encoding the protein of interest into an appropriate expression vector. As used herein, “expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes), retrotransposons (e.g. piggyback, sleeping beauty), and viruses (e.g., lentiviruses, retroviruses, adenoviruses, and adeno-associated viruses) that incorporate the recombinant polynucleotide of interest.

In certain embodiments, the expression vector is a viral vector. The term “virus” is used herein to refer to an obligate intracellular parasite having no protein-synthesizing or energy-generating mechanism. Exemplary viral vectors include retroviral vectors (e.g., lentiviral vectors), adenoviral vectors, adeno-associated viral vectors, herpesviruses vectors, epstein-barr virus (EBV) vectors, polyomavirus vectors (e.g. simian vacuolating virus 40 (SV10) vectors), poxvirus vectors, and pseudotype virus vectors.

The virus may be a RNA virus (having a genome that is composed of RNA) or a DNA virus (having a genome composed of DNA). In certain embodiments, the viral vector is a DNA virus vector. Exemplary DNA viruses include parvoviruses (e.g., adeno-associated viruses), adenoviruses, asfarviruses, herpesviruses (e.g., herpes simplex virus 1 and 2 (HSV-1 and HSV-2), epstein-barr virus (EBV), cytomegalovirus (CMV)), papillomoviruses (e.g., HPV), polyomaviruses (e.g., simian vacuolating virus 40 (SV40)), and poxviruses (e.g., vaccinia virus, cowpox virus, smallpox virus, fowlpox virus, sheeppox virus, myxoma virus). In certain embodiments, the viral vector is a RNA virus vector. Exemplary RNA viruses include bunyaviruses (e.g., hantavirus), coronaviruses, flaviviruses (e.g., yellow fever virus, west nile virus, dengue virus), hepatitis viruses (e.g., hepatitis A virus, hepatitis C virus, hepatitis E virus), influenza viruses (e.g., influenza virus type A, influenza virus type B, influenza virus type C), measles virus, mumps virus, noroviruses (e.g., Norwalk virus), poliovirus, respiratory syncytial virus (RSV), retroviruses (e.g., human immunodeficiency virus-1 (HIV-1)) and toroviruses.

In certain embodiments, the expression vector comprises a regulatory sequence or promoter operably linked to the nucleotide sequence encoding the protein of interest, e.g., a chimeric antigen receptor and/or a sialidase. The term “operably linked” refers to a linkage of polynucleotide elements in a functional relationship. A nucleic acid sequence is “operably linked” when it is placed into a functional relationship with another nucleic acid sequence. For instance, a promoter or enhancer is operably linked to a gene if it affects the transcription of the gene. Operably linked nucleotide sequences are typically contiguous. However, as enhancers generally function when separated from the promoter by several kilobases and intronic sequences may be of variable lengths, some polynucleotide elements may be operably linked but not directly flanked and may even function in trans from a different allele or chromosome.

Exemplary promoters which may be employed include, but are not limited to, the retroviral LTR, the SV40 promoter, the human cytomegalovirus (CMV) promoter, the U6 promoter, or any other promoter (e.g., cellular promoters such as eukaryotic cellular promoters including, but not limited to, the histone, pol III, and β-actin promoters). Other viral promoters which may be employed include, but are not limited to, adenovirus promoters, TK promoters, and B19 parvovirus promoters.

In certain embodiments, a promoter is an inducible promoter. The use of an inducible promoter allows for expression of an operatively linked polynucleotide sequence to be turned on or off when desired. In certain embodiments, the promoter is induced in the presence of an exogenous molecule or activity, e.g., a metallothionine promoter, a glucocorticoid promoter, a progesterone promoter, and a tetracycline promoter. In certain embodiments, the promoter is induced in the tumor microenvironment, e.g., an IL-2 promoter, a NFAT promoter, a cell surface protein promoter (e.g., a CD69 promoter or a PD-1 promoter), a cytokine promoter (e.g., a TNF promoter), a cellular activation promoter (e.g., a CTLA4, OX40, or CD40L promoter), or a cell surface adhesion protein promoter (e.g., a VLA-1 promoter).

In certain embodiments, a promoter mediates rapid, sustained expression, measured in days (e.g., a CD69 promoter). In certain embodiments, a promoter mediates delayed, late-inducible expression (e.g., a VLA1 promoter). In certain embodiments, a promoter mediates rapid, transient expression (e.g., a TNF promoter, an immediate early response gene promoter and others).

The selection of a promoter, e.g., strong, weak, inducible, tissue-specific, developmental-specific, having specific kinetics of activation (e.g., early and/or late activation), and/or having specific kinetics of expression of an induced gene (e.g., short or long expression) is within the ordinary skill of the artisan and will be apparent to those skilled in the art from the teachings contained herein.

Examples of other systems for expressing or regulating expression include “ON-Switch” CARs (Wu et al. (2015) SCIENCE 350: aab4077), combinatorial activation systems (Fedorov et al. (2014) CANCER JOURNAL 20:160-165; Kloss et al. (2013) NATURE BIOTECHNOLOGY 31: 71-75), doxycycline-inducible CARs (Sakemura et al. (2016) CANCER IMMUNOL. RES. 4:658-668), antibody-inducible CARs (Hill et al. (2018) NATURE CHEMICAL BIOLOGY 14:112-117), kill switches (Di Stasi et al. (2011) N. ENGL. J. MED. 365:1673-1683 (2011); Budde et al. (2013) PLOS ONE 8: e82742), pause switches (Wei et al. (2012) NATURE 488: 384-388), tunable receptor systems (Ma et al. (2016) PROC. NATL. ACAD. SCI. USA 113: E450-458; Rodgers et al. (2016) PROC. NATL. ACAD. SCI. USA 113: E459-468; Kudo et al. (2014) CANCER RES. 74: 93-103), and proliferation switches (Chen et al. (2010) PROC. NATL. ACAD. SCI. USA 107, 8531-8536).

### Lentivirus Vectors

In certain embodiments, the viral vector can be a retroviral vector. Examples of retroviral vectors include moloney murine leukemia virus vectors, spleen necrosis virus vectors, and vectors derived from retroviruses such as rous sarcoma virus, harvey sarcoma virus, avian leukosis virus, human immunodeficiency virus, myeloproliferative sarcoma virus, and mammary tumor virus. Retroviral vectors are useful as agents to mediate retroviral-mediated gene transfer into eukaryotic cells.

In certain embodiments, the retroviral vector is a lentiviral vector. Exemplary lentiviral vectors include vectors derived from human immunodeficiency virus-1 (HIV-1), human immunodeficiency virus-2 (HIV-2), simian immunodeficiency virus (SIV), feline immunodeficiency virus (FIV), bovine immunodeficiency virus (BIV), Jembrana Disease Virus (JDV), equine infectious anemia virus (EIAV), and caprine arthritis encephalitis virus (CAEV).

Retroviral vectors typically are constructed such that the majority of sequences coding for the structural genes of the virus are deleted and replaced by the gene(s) of interest. Often, the structural genes (i.e., gag, pol, and env), are removed from the retroviral backbone using genetic engineering techniques known in the art. Accordingly, a minimum retroviral vector comprises from 5′ to 3′: a 5′ long terminal repeat (LTR), a packaging signal, an optional exogenous promoter and/or enhancer, an exogenous gene of interest, and a 3′ LTR. If no exogenous promoter is provided, gene expression is driven by the 5′ LTR, which is a weak promoter and requires the presence of Tat to activate expression. The structural genes can be provided in separate vectors for manufacture of the lentivirus, rendering the produced virions replication-defective. Specifically, with respect to lentivirus, the packaging system may comprise a single packaging vector encoding the Gag, Pol, Rev, and Tat genes, and a third, separate vector encoding the envelope protein Env (usually VSV-G due to its wide infectivity). To improve the safety of the packaging system, the packaging vector can be split, expressing Rev from one vector, Gag and Pol from another vector. Tat can also be eliminated from the packaging system by using a retroviral vector comprising a chimeric 5′ LTR, wherein the U3 region of the 5′ LTR is replaced with a heterologous regulatory element.

The genes can be incorporated into the proviral backbone in several general ways. The most straightforward constructions are ones in which the structural genes of the retrovirus are replaced by a single gene that is transcribed under the control of the viral regulatory sequences within the LTR. Retroviral vectors have also been constructed which can introduce more than one gene into target cells. Usually, in such vectors one gene is under the regulatory control of the viral LTR, while the second gene is expressed either off a spliced message or is under the regulation of its own, internal promoter.

Accordingly, the new gene(s) are flanked by 5′ and 3′ LTRs, which serve to promote transcription and polyadenylation of the virion RNAs, respectively. The term “long terminal repeat” or “LTR” refers to domains of base pairs located at the ends of retroviral DNAs which, in their natural sequence context, are direct repeats and contain U3, R and U5 regions. LTRs generally provide functions fundamental to the expression of retroviral genes (e.g., promotion, initiation and polyadenylation of gene transcripts) and to viral replication. The LTR contains numerous regulatory signals including transcriptional control elements, polyadenylation signals, and sequences needed for replication and integration of the viral genome. The U3 region contains the enhancer and promoter elements. The U5 region is the sequence between the primer binding site and the R region and contains the polyadenylation sequence. The R (repeat) region is flanked by the U3 and U5 regions. In certain embodiments, the R region comprises a trans-activation response (TAR) genetic element, which interacts with the trans-activator (tat) genetic element to enhance viral replication. This element is not required in embodiments wherein the U3 region of the 5′ LTR is replaced by a heterologous promoter.

In certain embodiments, the retroviral vector comprises a modified 5′ LTR and/or 3′ LTR. Modifications of the 3′ LTR are often made to improve the safety of lentiviral or retroviral systems by rendering viruses replication-defective. In specific embodiments, the retroviral vector is a self-inactivating (SIN) vector. As used herein, a SIN retroviral vector refers to a replication-defective retroviral vector in which the 3′ LTR U3 region has been modified (e.g., by deletion or substitution) to prevent viral transcription beyond the first round of viral replication. This is because the 3′ LTR U3 region is used as a template for the 5′ LTR U3 region during viral replication and, thus, the viral transcript cannot be made without the U3 enhancer-promoter. In a further embodiment, the 3′ LTR is modified such that the U5 region is replaced, for example, with an ideal polyadenylation sequence. It should be noted that modifications to the LTRs such as modifications to the 3′ LTR, the 5′ LTR, or both 3′ and 5′ LTRs, are also included in the invention.

In certain embodiments, the U3 region of the 5′ LTR is replaced with a heterologous promoter to drive transcription of the viral genome during production of viral particles. Examples of heterologous promoters which can be used include, for example, viral simian virus 40 (SV40) (e.g., early or late), cytomegalovirus (CMV) (e.g., immediate early), Moloney murine leukemia virus (MoMLV), Rous sarcoma virus (RSV), and herpes simplex virus (HSV) (thymidine kinase) promoters. Typical promoters are able to drive high levels of transcription in a Tat-independent manner. This replacement reduces the possibility of recombination to generate replication-competent virus, because there is no complete U3 sequence in the virus production system.

Adjacent the 5′ LTR are sequences necessary for reverse transcription of the genome and for efficient packaging of viral RNA into particles (the Psi site). As used herein, the term “packaging signal” or “packaging sequence” refers to sequences located within the retroviral genome which are required for encapsidation of retroviral RNA strands during viral particle formation (see e.g., Clever et al., 1995 J. VIROLOGY, 69(4):2101-09). The packaging signal may be a minimal packaging signal (also referred to as the psi [Ψ] sequence) needed for encapsidation of the viral genome.

In certain embodiments, the retroviral vector (e.g., lentiviral vector) further comprises a FLAP. As used herein, the term “FLAP” refers to a nucleic acid whose sequence includes the central polypurine tract and central termination sequences (cPPT and CTS) of a retrovirus, e.g., HIV-1 or HIV-2. Suitable FLAP elements are described in U.S. Pat. No. 6,682,907 and in Zennou et al. (2000) CELL, 101:173. During reverse transcription, central initiation of the plus-strand DNA at the cPPT and central termination at the CTS lead to the formation of a three-stranded DNA structure: a central DNA flap. While not wishing to be bound by any theory, the DNA flap may act as a cis-active determinant of lentiviral genome nuclear import and/or may increase the titer of the virus. In particular embodiments, the retroviral vector backbones comprise one or more FLAP elements upstream or downstream of the heterologous genes of interest in the vectors. For example, in particular embodiments, a transfer plasmid includes a FLAP element. In one embodiment, a vector of the invention comprises a FLAP element isolated from HIV-1.

In certain embodiments, the retroviral vector (e.g., lentiviral vector) further comprises an export element. In one embodiment, retroviral vectors comprise one or more export elements. The term “export element” refers to a cis-acting post-transcriptional regulatory element which regulates the transport of an RNA transcript from the nucleus to the cytoplasm of a cell. Examples of RNA export elements include, but are not limited to, the human immunodeficiency virus (HIV) RRE (see e.g., Cullen et al., (1991) J. VIROL. 65: 1053; and Cullen et al., (1991) CELL 58: 423) and the hepatitis B virus post-transcriptional regulatory element (HPRE). Generally, the RNA export element is placed within the 3′ UTR of a gene, and can be inserted as one or multiple copies.

In certain embodiments, the retroviral vector (e.g., lentiviral vector) further comprises a posttranscriptional regulatory element. A variety of posttranscriptional regulatory elements can increase expression of a heterologous nucleic acid, e.g., woodchuck hepatitis virus posttranscriptional regulatory element (WPRE; see Zufferey et al., (1999) J. VIROL., 73:2886); the posttranscriptional regulatory element present in hepatitis B virus (HPRE) (Huang et al., MOL. CELL. BIOL., 5:3864); and the like (Liu et al., (1995), GENES DEV., 9:1766). The posttranscriptional regulatory element is generally positioned at the 3′ end the heterologous nucleic acid sequence. This configuration results in synthesis of an mRNA transcript whose 5′ portion comprises the heterologous nucleic acid coding sequences and whose 3′ portion comprises the posttranscriptional regulatory element sequence. In certain embodiments, vectors of the invention lack or do not comprise a posttranscriptional regulatory element such as a WPRE or HPRE, because in some instances these elements increase the risk of cellular transformation and/or do not substantially or significantly increase the amount of mRNA transcript or increase mRNA stability. Therefore, in certain embodiments, vectors of the invention lack or do not comprise a WPRE or HPRE as an added safety measure.

Elements directing the efficient termination and polyadenylation of the heterologous nucleic acid transcripts increase heterologous gene expression. Transcription termination signals are generally found downstream of the polyadenylation signal. Accordingly, in certain embodiments, the retroviral vector (e.g., lentiviral vector) further comprises a polyadenylation signal. The term “polyadenylation signal” or “polyadenylation sequence” as used herein denotes a DNA sequence which directs both the termination and polyadenylation of the nascent RNA transcript by RNA polymerase H. Efficient polyadenylation of the recombinant transcript is desirable as transcripts lacking a polyadenylation signal are unstable and are rapidly degraded. Illustrative examples of polyadenylation signals that can be used in a vector of the invention, includes an ideal polyadenylation sequence (e.g., AATAAA, ATTAAA AGTAAA (SEQ ID NO: 161)), a bovine growth hormone polyadenylation sequence (BGHpA), a rabbit β-globin polyadenylation sequence (rpgpA), or another suitable heterologous or endogenous polyadenylation sequence known in the art.

In certain embodiments, a retroviral vector further comprises an insulator element. Insulator elements may contribute to protecting retrovirus-expressed sequences, e.g., therapeutic genes, from integration site effects, which may be mediated by cis-acting elements present in genomic DNA and lead to deregulated expression of transferred sequences (i.e., position effect; see, e.g., Burgess-Beusse et al., (2002) PROC. NATL. ACAD. SCI., USA, 99:16433; and Zhan et al., 2001, HUM. GENET., 109:471). In certain embodiments, the retroviral vector comprises an insulator element in one or both LTRs or elsewhere in the region of the vector that integrates into the cellular genome. Suitable insulators for use in the invention include, but are not limited to, the chicken β-globin insulator (see Chung et al., (1993). CELL 74:505; Chung et al., (1997) PROC. NATL. ACAD. SCI., USA 94:575; and Bell et al., 1999. CELL 98:387). Examples of insulator elements include, but are not limited to, an insulator from a β-globin locus, such as chicken HS4.

Non-limiting examples of lentiviral vectors include pLVX-EF1alpha-AcGFP1-C1 (Clontech Catalog #631984), pLVX-EF1alpha-IRES-mCherry (Clontech Catalog #631987), pLVX-Puro (Clontech Catalog #632159), pLVX-IRES-Puro (Clontech Catalog #632186), pLenti6/V5-DEST™ (Thermo Fisher), pLenti6.2/V5-DEST™ (Thermo Fisher), pLKO.1 (Plasmid #10878 at Addgene), pLKO.3G (Plasmid #14748 at Addgene), pSico (Plasmid #11578 at Addgene), pLJM1-EGFP (Plasmid #19319 at Addgene), FUGW (Plasmid #14883 at Addgene), pLVTHM (Plasmid #12247 at Addgene), pLVUT-tTR-KRAB (Plasmid #11651 at Addgene), pLL3.7 (Plasmid #11795 at Addgene), pLB (Plasmid #11619 at Addgene), pWPXL (Plasmid #12257 at Addgene), pWPI (Plasmid #12254 at Addgene), EF.CMV.RFP (Plasmid #17619 at Addgene), pLenti CMV Puro DEST (Plasmid #17452 at Addgene), pLenti-puro (Plasmid #39481 at Addgene), pULTRA (Plasmid #24129 at Addgene), pLX301 (Plasmid #25895 at Addgene), pHIV-EGFP (Plasmid #21373 at Addgene), pLV-mCherry (Plasmid #36084 at Addgene), pLionII (Plasmid #1730 at Addgene), pInducer10-mir-RUP-PheS (Plasmid #44011 at Addgene). These vectors can be modified to be suitable for therapeutic use. For example, a selection marker (e.g., puro, EGFP, or mCherry) can be deleted or replaced with a second exogenous gene of interest. Further examples of lentiviral vectors are disclosed in U.S. Pat. Nos. 7,629,153, 7,198,950, 8,329,462, 6,863,884, 6,682,907, 7,745,179, 7,250,299, 5,994,136, 6,287,814, 6,013,516, 6,797,512, 6,544,771, 5,834,256, 6,958,226, 6,207,455, 6,531,123, and 6,352,694, and PCT Publication No. WO2017/091786.

### Adeno-Associated Virus (AAV) Vectors

In certain embodiments, an expression vector is an adeno-associated virus (AAV) vector. AAV is a small, nonenveloped icosahedral virus of the genus Dependoparvovirus and family Parvovirus. AAV has a single-stranded linear DNA genome of approximately 4.7 kb. AAV is capable of infecting both dividing and quiescent cells of several tissue types, with different AAV serotypes exhibiting different tissue tropism.

AAV includes numerous serologically distinguishable types including serotypes AAV-1 to AAV-12, as well as more than 100 serotypes from nonhuman primates (See, e.g., Srivastava (2008) J. CELL BIOCHEM., 105(1): 17-24, and Gao et al. (2004) J. VIROL., 78(12), 6381-6388). The serotype of the AAV vector used in the present invention can be selected by a skilled person in the art based on the efficiency of delivery, tissue tropism, and immunogenicity. For example, AAV-1, AAV-2, AAV-4, AAV-5, AAV-8, and AAV-9 can be used for delivery to the central nervous system; AAV-1, AAV-8, and AAV-9 can be used for delivery to the heart; AAV-2 can be used for delivery to the kidney; AAV-7, AAV-8, and AAV-9 can be used for delivery to the liver; AAV-4, AAV-5, AAV-6, AAV-9 can be used for delivery to the lung, AAV-8 can be used for delivery to the pancreas, AAV-2, AAV-5, and AAV-8 can be used for delivery to the photoreceptor cells; AAV-1, AAV-2, AAV-4, AAV-5, and AAV-8 can be used for delivery to the retinal pigment epithelium; AAV-1, AAV-6, AAV-7, AAV-8, and AAV-9 can be used for delivery to the skeletal muscle. In certain embodiments, the AAV capsid protein comprises a sequence as disclosed in U.S. Pat. No. 7,198,951, such as, but not limited to, AAV-9 (SEQ ID NOs: 1-3 of U.S. Pat. No. 7,198,951), AAV-2 (SEQ ID NO: 4 of U.S. Pat. No. 7,198,951), AAV-1 (SEQ ID NO: 5 of U.S. Pat. No. 7,198,951), AAV-3 (SEQ ID NO: 6 of U.S. Pat. No. 7,198,951), and AAV-8 (SEQ ID NO: 7 of U.S. Pat. No. 7,198,951). AAV serotypes identified from rhesus monkeys, e.g., rh.8, rh.10, rh.39, rh.43, and rh.74, are also contemplated in the instant invention. Besides the natural AAV serotypes, modified AAV capsids have been developed for improving efficiency of delivery, tissue tropism, and immunogenicity. Exemplary natural and modified AAV capsids are disclosed in U.S. Pat. Nos. 7,906,111, 9,493,788, and 7,198,951, and PCT Publication No. WO2017189964A2.

The wild-type AAV genome contains two 145 nucleotide inverted terminal repeats (ITRs), which contain signal sequences directing AAV replication, genome encapsidation and integration. In addition to the ITRs, three AAV promoters, p5, p19, and p40, drive expression of two open reading frames encoding rep and cap genes. Two rep promoters, coupled with differential splicing of the single AAV intron, result in the production of four rep proteins (Rep 78, Rep 68, Rep 52, and Rep 40) from the rep gene. Rep proteins are responsible for genomic replication. The Cap gene is expressed from the p40 promoter, and encodes three capsid proteins (VP1, VP2, and VP3) which are splice variants of the cap gene. These proteins form the capsid of the AAV particle.

Because the cis-acting signals for replication, encapsidation, and integration are contained within the ITRs, some or all of the 4.3 kb internal genome may be replaced with foreign DNA, for example, an expression cassette for an exogenous gene of interest. Accordingly, in certain embodiments, the AAV vector comprises a genome comprising an expression cassette for an exogenous gene flanked by a 5′ ITR and a 3′ ITR. The ITRs may be derived from the same serotype as the capsid or a derivative thereof. Alternatively, the ITRs may be of a different serotype from the capsid, thereby generating a pseudotyped AAV. In certain embodiments, the ITRs are derived from AAV-2. In certain embodiments, the ITRs are derived from AAV-5. At least one of the ITRs may be modified to mutate or delete the terminal resolution site, thereby allowing production of a self-complementary AAV vector.

The rep and cap proteins can be provided in trans, for example, on a plasmid, to produce an AAV vector. A host cell line permissive of AAV replication must express the rep and cap genes, the ITR-flanked expression cassette, and helper functions provided by a helper virus, for example adenoviral genes Ela, E1b55K, E2a, E4orf6, and VA (Weitzman et al., Adeno-associated virus biology. Adeno-Associated Virus: Methods and Protocols, pp. 1-23, 2011). Methods for generating and purifying AAV vectors have been described in detail (See e.g., Mueller et al., (2012) CURRENT PROTOCOLS IN MICROBIOLOGY, 14D.1.1-14D.1.21, Production and Discovery of Novel Recombinant Adeno-Associated Viral Vectors). Numerous cell types are suitable for producing AAV vectors, including HEK293 cells, COS cells, HeLa cells, BHK cells, Vero cells, as well as insect cells (See e.g. U.S. Pat. Nos. 6,156,303, 5,387,484, 5,741,683, 5,691,176, 5,688,676, and 8,163,543, U.S. Patent Publication No. 20020081721, and PCT Publication Nos. WO00/47757, WO00/24916, and WO96/17947). AAV vectors are typically produced in these cell types by one plasmid containing the ITR-flanked expression cassette, and one or more additional plasmids providing the additional AAV and helper virus genes.

AAV of any serotype may be used in the present invention. Similarly, it is contemplated that any adenoviral type may be used, and a person of skill in the art will be able to identify AAV and adenoviral types suitable for the production of their desired recombinant AAV vector (rAAV). AAV particles may be purified, for example by affinity chromatography, iodixonal gradient, or CsCl gradient.

AAV vectors may have single-stranded genomes that are 4.7 kb in size, or are larger or smaller than 4.7 kb, including oversized genomes that are as large as 5.2 kb, or as small as 3.0 kb. Thus, where the exogenous gene of interest to be expressed from the AAV vector is small, the AAV genome may comprise a stuffer sequence. Further, vector genomes may be substantially self-complementary thereby allowing for rapid expression in the cell. In certain embodiments, the genome of a self-complementary AAV vector comprises from 5′ to 3′: a 5′ ITR; a first nucleic acid sequence comprising a promoter and/or enhancer operably linked to a coding sequence of a gene of interest; a modified ITR that does not have a functional terminal resolution site; a second nucleic acid sequence complementary or substantially complementary to the first nucleic acid sequence; and a 3′ ITR. AAV vectors containing genomes of all types are suitable for use in the method of the present invention.

Non-limiting examples of AAV vectors include pAAV-MCS (Agilent Technologies), pAAVK-EF1α-MCS (System Bio Catalog #AAV502A-1), pAAVK-EF1α-MCS1-CMV-MCS2 (System Bio Catalog #AAV503A-1), pAAV-ZsGreen1 (Clontech Catalog #6231), pAAV-MCS2 (Addgene Plasmid #46954), AAV-Stuffer (Addgene Plasmid #106248), pAAVscCBPIGpluc (Addgene Plasmid #35645), AAVS1_Puro_PGK1_3×FLAG_Twin_Strep (Addgene Plasmid #68375), pAAV-RAM-d2TTA::TRE-MCS-WPRE-pA (Addgene Plasmid #63931), pAAV-UbC (Addgene Plasmid #62806), pAAVS1-P-MCS (Addgene Plasmid #80488), pAAV-Gateway (Addgene Plasmid #32671), pAAV-Puro_siKD (Addgene Plasmid #86695), pAAVS1-Nst-MCS (Addgene Plasmid #80487), pAAVS1-Nst-CAG-DEST (Addgene Plasmid #80489), pAAVS1-P-CAG-DEST (Addgene Plasmid #80490), pAAVf-EnhCB-lacZnls (Addgene Plasmid #35642), and pAAVS1-shRNA (Addgene Plasmid #82697). These vectors can be modified to be suitable for therapeutic use. For example, an exogenous gene of interest can be inserted in a multiple cloning site, and a selection marker (e.g., puro or a gene encoding a fluorescent protein) can be deleted or replaced with another (same or different) exogenous gene of interest. Further examples of AAV vectors are disclosed in U.S. Pat. Nos. 5,871,982, 6,270,996, 7,238,526, 6,943,019, 6,953,690, 9,150,882, and 8,298,818, U.S. Patent Publication No. 2009/0087413, and PCT Publication Nos. WO2017075335A1, WO2017075338A2, and WO2017201258A1.

### Adenoviral Vectors

In certain embodiments, the viral vector can be an adenoviral vector. Adenoviruses are medium-sized (90-100 nm), non-enveloped (naked), icosahedral viruses composed of a nucleocapsid and a double-stranded linear DNA genome. The term “adenovirus” refers to any virus in the genus Adenoviridiae including, but not limited to, human, bovine, ovine, equine, canine, porcine, murine, and simian adenovirus subgenera. Typically, an adenoviral vector is generated by introducing one or more mutations (e.g., a deletion, insertion, or substitution) into the adenoviral genome of the adenovirus so as to accommodate the insertion of a non-native nucleic acid sequence, for example, for gene transfer, into the adenovirus.

A human adenovirus can be used as the source of the adenoviral genome for the adenoviral vector. For instance, an adenovirus can be of subgroup A (e.g., serotypes 12, 18, and 31), subgroup B (e.g., serotypes 3, 7, 11, 14, 16, 21, 34, 35, and 50), subgroup C (e.g., serotypes 1, 2, 5, and 6), subgroup D (e.g., serotypes 8, 9, 10, 13, 15, 17, 19, 20, 22-30, 32, 33, 36-39, and 42-48), subgroup E (e.g., serotype 4), subgroup F (e.g., serotypes 40 and 41), an unclassified serogroup (e.g., serotypes 49 and 51), or any other adenoviral serogroup or serotype. Adenoviral serotypes 1 through 51 are available from the American Type Culture Collection (ATCC, Manassas, Virginia). Non-group C adenoviral vectors, methods of producing non-group C adenoviral vectors, and methods of using non-group C adenoviral vectors are disclosed in, for example, U.S. Pat. Nos. 5,801,030, 5,837,511, and 5,849,561, and PCT Publication Nos. WO1997/012986 and WO1998/053087.

Non-human adenovirus (e.g., ape, simian, avian, canine, ovine, or bovine adenoviruses) can be used to generate the adenoviral vector (i.e., as a source of the adenoviral genome for the adenoviral vector). For example, the adenoviral vector can be based on a simian adenovirus, including both new world and old world monkeys (see, e.g., Virus Taxonomy: VHIth Report of the International Committee on Taxonomy of Viruses (2005)). A phylogeny analysis of adenoviruses that infect primates is disclosed in, e.g., Roy et al. (2009) PLoS PATHOG. 5(7):e1000503. A gorilla adenovirus can be used as the source of the adenoviral genome for the adenoviral vector. Gorilla adenoviruses and adenoviral vectors are described in, e.g., PCT Publication Nos. WO2013/052799, WO2013/052811, and WO2013/052832. The adenoviral vector can also comprise a combination of subtypes and thereby be a “chimeric” adenoviral vector.

The adenoviral vector can be replication-competent, conditionally replication-competent, or replication-deficient. A replication-competent adenoviral vector can replicate in typical host cells, i.e., cells typically capable of being infected by an adenovirus. A conditionally-replicating adenoviral vector is an adenoviral vector that has been engineered to replicate under pre-determined conditions. For example, replication-essential gene functions, e.g., gene functions encoded by the adenoviral early regions, can be operably linked to an inducible, repressible, or tissue-specific transcription control sequence, e.g., a promoter. Conditionally-replicating adenoviral vectors are further described in U.S. Pat. No. 5,998,205. A replication-deficient adenoviral vector is an adenoviral vector that requires complementation of one or more gene functions or regions of the adenoviral genome that are required for replication, as a result of, for example, a deficiency in one or more replication-essential gene function or regions, such that the adenoviral vector does not replicate in typical host cells, especially those in a human to be infected by the adenoviral vector.

Preferably, the adenoviral vector is replication-deficient, such that the replication-deficient adenoviral vector requires complementation of at least one replication-essential gene function of one or more regions of the adenoviral genome for propagation (e.g., to form adenoviral vector particles). The adenoviral vector can be deficient in one or more replication-essential gene functions of only the early regions (i.e., E1-E4 regions) of the adenoviral genome, only the late regions (i.e., L1-L5 regions) of the adenoviral genome, both the early and late regions of the adenoviral genome, or all adenoviral genes (i.e., a high capacity adenovector (HC-Ad)). See, e.g., Morsy et al. (1998) PROC. NATL. ACAD. SCI. USA 95: 965-976, Chen et al. (1997) PROC. NATL. ACAD. SCI. USA 94: 1645-1650, and Kochanek et al. (1999) HUM. GENE THER. 10(15):2451-9. Examples of replication-deficient adenoviral vectors are disclosed in U.S. Pat. Nos. 5,837,511, 5,851,806, 5,994,106, 6,127,175, 6,482,616, and 7,195,896, and PCT Publication Nos. WO1994/028152, WO1995/002697, WO1995/016772, WO1995/034671, WO1996/022378, WO1997/012986, WO1997/021826, and WO2003/022311.

The replication-deficient adenoviral vector of the invention can be produced in complementing cell lines that provide gene functions not present in the replication-deficient adenoviral vector, but required for viral propagation, at appropriate levels in order to generate high titers of viral vector stock. Such complementing cell lines are known and include, but are not limited to, 293 cells (described in, e.g., Graham et al. (1977) J. GEN. VIROL. 36: 59-72), PER.C6 cells (described in, e.g., PCT Publication No. WO1997/000326, and U.S. Pat. Nos. 5,994,128 and 6,033,908), and 293-ORF6 cells (described in, e.g., PCT Publication No. WO1995/034671 and Brough et al. (1997) J. VIROL. 71: 9206-9213). Other suitable complementing cell lines to produce the replication-deficient adenoviral vector of the invention include complementing cells that have been generated to propagate adenoviral vectors encoding transgenes whose expression inhibits viral growth in host cells (see, e.g., U.S. Patent Publication No. 2008/0233650). Additional suitable complementing cells are described in, for example, U.S. Pat. Nos. 6,677,156 and 6,682,929, and PCT Publication No. WO2003/020879. Formulations for adenoviral vector-containing compositions are further described in, for example, U.S. Pat. Nos. 6,225,289, and 6,514,943, and PCT Publication No. WO2000/034444.

Additional exemplary adenoviral vectors, and/or methods for making or propagating adenoviral vectors are described in U.S. Pat. Nos. 5,559,099, 5,837,511, 5,846,782, 5,851,806, 5,994,106, 5,994,128, 5,965,541, 5,981,225, 6,040,174, 6,020,191, 6,083,716, 6,113,913, 6,303,362, 7,067,310, and 9,073,980.

Commercially available adenoviral vector systems include the ViraPower™ Adenoviral Expression System available from Thermo Fisher Scientific, the AdEasy™ adenoviral vector system available from Agilent Technologies, and the Adeno-X™ Expression System 3 available from Takara Bio USA, Inc.

### Viral Vector Production

Methods for producing viral vectors are known in the art. Typically, a virus of interest is produced in a suitable host cell line using conventional techniques including culturing a transfected or infected host cell under suitable conditions so as to allow the production of infectious viral particles. Nucleic acids encoding viral genes and/or genes of interest can be incorporated into plasmids and introduced into host cells through conventional transfection or transformation techniques. Exemplary suitable host cells for production of disclosed viruses include human cell lines such as HeLa, Hela-S3, HEK293, 911, A549, HER96, or PER-C6 cells. Specific production and purification conditions will vary depending upon the virus and the production system employed.

In certain embodiments, producer cells may be directly administered to a subject, however, in other embodiments, following production, infectious viral particles are recovered from the culture and optionally purified. Typical purification steps may include plaque purification, centrifugation, e.g., cesium chloride gradient centrifugation, clarification, enzymatic treatment, e.g., benzonase or protease treatment, chromatographic steps, e.g., ion exchange chromatography or filtration steps.

### IV. Pretreated Immune Cells

An immune cell (for example, an isolated naturally occurring immune cell or an engineered immune cell described herein) may, for example, be pretreated with a sialidase (for example, a sialidase described herein), prior to administration to a subject. It is contemplated that pretreatment of an immune cell with a sialidase may remove sialic acid and/or sialic acid containing molecules from the surface of the immune cell, thereby reducing immune inhibition mediated by the sialic acid and/or sialic acid containing molecules and enhancing a treatment using the immune cell.

The invention provides a pharmaceutical composition comprising: (a) an isolated immune cell pretreated with a sialidase; and (b) a pharmaceutically acceptable carrier or diluent, as well as a method of treating cancer in a subject in need thereof, where the method comprises administering to the subject an isolated immune cell, for example, a T-cell, pretreated with a sialidase.

The immune cell may be pretreated with the sialidase for at least 30 minutes, at least 1 hour, at least 2 hours, at least 3 hours, at least 6 hours, at least 12 hours, at least 1 day, at least 2 days, at least 3 days, at least 4 days, at least 5 days, at least 6 days, or at least 1 week prior to administration to a subject.

Depending upon the circumstances, the immune cell may be used as is (namely, as a cell preparation containing the sialidase) or after purification from the sialidase. If purification is desired, the immune cell may be purified from the sialidase by any method known in the art, including, for example, by harvesting the cells as a cell pellet via centrifugation together with washing, for example, with a buffered solution, or by immunoprecipitation or affinity purification. An exemplary anti-Neu2 antibody suitable for removal of Neu2 by immunoprecipitation or affinity purification is 3B9 available from Novus Biologicals.

In certain embodiments, the purification results in removal of at least 10%, at least 20%, at least 30%, at least 40%, at least 50%, at least 60%, at least 70%, at least 80%, at least 90%, or 100% of the sialidase activity present prior to purification. In certain embodiments, following purification, a composition comprising the immune cell has no detectable sialidase activity. Sialidase activity may be assayed by any method known in the art, including, for example, by measuring the release of sialic acid from the fluorogenic substrate 4-methylumbelliferyl-N-acetylneuraminic acid (4MU-NeuAc). In certain embodiments, following purification, incubation of the composition with 2000 μM 4MU-NeuAc at pH 7 results in a fluorescent signal (A.U.) of less than 3.0×10⁷, 2.0×10⁷, 1.0×10⁷, 0.5×10⁷, 0.25×10⁷, or 0.1×10⁷.

### V. Pharmaceutical Compositions

For therapeutic use, an immune cell (for example, an isolated naturally occurring immune cell or an engineered immune cell described herein) and/or a sialidase preferably is combined with a pharmaceutically acceptable carrier. The term “pharmaceutically acceptable” as used herein refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “pharmaceutically acceptable carrier” as used herein refers to buffers, carriers, and excipients suitable for use in contact with the tissues of human beings and animals without excessive toxicity, irritation, allergic response, or other problem or complication, commensurate with a reasonable benefit/risk ratio. Pharmaceutically acceptable carriers include any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see, e.g., Martin, Remington's Pharmaceutical Sciences, 15th Ed., Mack Publ. Co., Easton, PA [1975]. Pharmaceutically acceptable carriers include buffers, solvents, dispersion media, coatings, isotonic and absorption delaying agents, and the like, that are compatible with pharmaceutical administration. The use of such media and agents for pharmaceutically active substances is known in the art.

In certain embodiments, a pharmaceutical composition may contain formulation materials for modifying, maintaining or preserving, for example, the pH, osmolarity, viscosity, clarity, color, isotonicity, odor, sterility, stability, rate of dissolution or release, adsorption or penetration of the composition. In such embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants (See Remington's Pharmaceutical Sciences, 18th ed. (Mack Publishing Company, 1990).

In certain embodiments, a pharmaceutical composition may contain nanoparticles, e.g., polymeric nanoparticles, liposomes, or micelles (See Anselmo et al. (2016) BIOENG. TRANSL. MED. 1: 10-29).

In certain embodiments, a pharmaceutical composition may contain a sustained- or controlled-delivery formulation. Techniques for formulating sustained- or controlled-delivery means, such as liposome carriers, bio-erodible microparticles or porous beads and depot injections, are also known to those skilled in the art. Sustained-release preparations may include, e.g., porous polymeric microparticles or semipermeable polymer matrices in the form of shaped articles, e.g., films, or microcapsules. Sustained release matrices may include polyesters, hydrogels, polylactides, copolymers of L-glutamic acid and gamma ethyl-L-glutamate, poly (2-hydroxyethyl-inethacrylate), ethylene vinyl acetate, or poly-D(−)-3-hydroxybutyric acid. Sustained release compositions may also include liposomes that can be prepared by any of several methods known in the art.

Pharmaceutical compositions containing an immune cell and/or a sialidase disclosed herein can be presented in a dosage unit form and can be prepared by any suitable method. A pharmaceutical composition should be formulated to be compatible with its intended route of administration. Examples of routes of administration are intravenous (IV), intradermal, inhalation, transdermal, topical, transmucosal, intrathecal and rectal administration. In certain embodiments, a pharmaceutical composition containing an immune cell and/or a sialidase disclosed herein is administered by IV infusion. In certain embodiments, a pharmaceutical composition containing an immune cell and/or a sialidase disclosed herein is administered by intratumoral injection.

Useful formulations can be prepared by methods known in the pharmaceutical art. For example, see Remington's Pharmaceutical Sciences, 18th ed. (Mack Publishing Company, 1990). Formulation components suitable for parenteral administration include a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as EDTA; buffers such as acetates, citrates or phosphates; and agents for the adjustment of tonicity such as sodium chloride or dextrose.

For intravenous administration, suitable carriers include physiological saline, bacteriostatic water, Cremophor ELTM (BASF, Parsippany, NJ) or phosphate buffered saline (PBS). The carrier should be stable under the conditions of manufacture and storage, and should be preserved against microorganisms. The carrier can be a solvent or dispersion medium containing, for example, water, ethanol, polyol (for example, glycerol, propylene glycol, and liquid polyetheylene glycol), and suitable mixtures thereof.

Pharmaceutical formulations preferably are sterile. Sterilization can be accomplished by any suitable method, e.g., filtration through sterile filtration membranes. Where the composition is lyophilized, filter sterilization can be conducted prior to or following lyophilization and reconstitution.

A therapeutically effective amount of isolated, naturally occurring or engineered immune cells, e.g., T-cells, is in the range of, e.g., 10⁵ to 10⁹ cells, 10⁵ to 10⁸ cells, 10⁵ to 10⁷ cells, 10⁵ to 10⁶ cells, 10⁶ to 10⁹ cells, 10⁶ to 10⁸ cells, 10⁶ to 10⁷ cells, 10⁷ to 10⁹ cells, 10⁷ to 10⁸ cells, or 10⁸ to 10⁹ cells per kilogram. The amount administered will depend on variables such as the type and extent of disease or indication to be treated, the overall health of the patient, the in vivo potency of the antibody, the pharmaceutical formulation, and the route of administration. Progress can be monitored by periodic assessment.

The cell compositions described herein may be administered locally or systemically. Administration will generally be parenteral administration. In a preferred embodiment, the pharmaceutical composition is administered subcutaneously and in an even more preferred embodiment intravenously. Preparations for parenteral administration include sterile aqueous or non-aqueous solutions, suspensions, and emulsions.

Generally, a therapeutically effective amount of active component, for example, a sialidase, is in the range of 0.1 mg/kg to 100 mg/kg, e.g., 1 mg/kg to 100 mg/kg, 1 mg/kg to 10 mg/kg. The amount administered will depend on variables such as the type and extent of disease or indication to be treated, the overall health of the patient, the in vivo potency of the antibody, the pharmaceutical formulation, and the route of administration. The initial dosage can be increased beyond the upper level in order to rapidly achieve the desired blood-level or tissue-level. Alternatively, the initial dosage can be smaller than the optimum, and the daily dosage may be progressively increased during the course of treatment. Human dosage can be optimized, e.g., in a conventional Phase I dose escalation study designed to run from 0.5 mg/kg to 20 mg/kg. Dosing frequency can vary, depending on factors such as route of administration, dosage amount, serum half-life of the antibody, and the disease being treated. Exemplary dosing frequencies are once per day, once per week and once every two weeks. A preferred route of administration is parenteral, e.g., intravenous infusion. In certain embodiments, a sialidase is lyophilized, and then reconstituted in buffered saline, at the time of administration.

### VI. Therapeutic Uses

The compositions and methods disclosed herein can be used to treat various forms of cancer in a subject or inhibit cancer growth in a subject. The invention provides a method of treating a cancer in a subject. The method comprises administering to the subject an effective amount of a disclosed immune cell and/or sialidase, either alone or in a combination with another therapeutic agent to treat the cancer in the subject. The term “effective amount” as used herein refers to the amount of an active agent (e.g., an immune cell and/or sialidase of the present invention) sufficient to effect beneficial or desired results. An effective amount can be administered in one or more administrations, applications or dosages and is not intended to be limited to a particular formulation or administration route.

As used herein, “treat”, “treating” and “treatment” mean the treatment of a disease in a subject, e.g., in a human. This includes: (a) inhibiting the disease, i.e., arresting its development; and (b) relieving the disease, i.e., causing regression of the disease state. As used herein, the terms “subject” and “patient” refer to an organism to be treated by the methods and compositions described herein. Such organisms preferably include, but are not limited to, mammals (e.g., murines, simians, equines, bovines, porcines, canines, felines, and the like), and more preferably includes humans.

Examples of cancers include solid tumors, soft tissue tumors, hematopoietic tumors and metastatic lesions. Examples of hematopoietic tumors include, leukemia, acute leukemia, acute lymphoblastic leukemia (ALL), B-cell, T-cell or FAB ALL, acute myeloid leukemia (AML), chronic myelocytic leukemia (CMIL), chronic lymphocytic leukemia (CLL), e.g., transformed CLL, diffuse large B-cell lymphomas (DLBCL), follicular lymphoma, hairy cell leukemia, myelodyplastic syndrome (MDS), a lymphoma, Hodgkin's disease, a malignant lymphoma, non-Hodgkin's lymphoma, Burkitt's lymphoma, multiple myeloma, or Richter's Syndrome (Richter's Transformation). Examples of solid tumors include malignancies, e.g., sarcomas, adenocarcinomas, and carcinomas, of the various organ systems, such as those affecting head and neck (including pharynx), thyroid, lung (small cell or non-small cell lung carcinoma (NSCLC)), breast, lymphoid, gastrointestinal (e.g., oral, esophageal, stomach, liver, pancreas, small intestine, colon and rectum, anal canal), genitals and genitourinary tract (e.g., renal, urothelial, bladder, ovarian, uterine, cervical, endometrial, prostate, testicular), CNS (e.g., neural or glial cells, e.g., neuroblastoma or glioma), or skin (e.g., melanoma).

In certain embodiments the cancer is an epithelial cancer, e.g., an epithelial cancer that upregulates the expression of sialylated glycans. Exemplary epithelial cancers include, but are not limited to, endometrial cancer, colon, ovarian cancer, cervical cancer, vulvar cancer, uterine cancer or fallopian tube cancer, breast cancer, prostate cancer, lung cancer, pancreatic cancer, urinary cancer, bladder cancer, head and neck cancer, oral cancer and liver cancer. Epithelial cancers also include carcinomas, for example, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, baso squamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniforni carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, and carcinoma villosum.

In certain embodiments, the cancer is an adenocarcinoma, a metastatic cancer and/or is a refractory cancer. In certain embodiments, the cancer is a breast, colon or colorectal, lung, ovarian, pancreatic, prostate, cervical, endometrial, head and neck, liver, renal, skin, stomach, testicular, thyroid or urothelial cancer. In certain embodiments, the cancer is an epithelial cancer, e.g., an endometrial cancer, ovarian cancer, cervical cancer, vulvar cancer, uterine cancer, fallopian tube cancer, breast cancer, prostate cancer, lung cancer, pancreatic cancer, urinary cancer, bladder cancer, head and neck cancer, oral cancer or liver cancer.

In certain embodiments, the cancer is a hematologic cancer, e.g., a leukemia, lymphoma, or multiple myeloma, e.g., Chronic lymphocytic leukemia (CLL), Acute myeloid leukemia (AML), Chronic myelogenous leukemia (CML), Non-Hodgkin lymphoma (NHL), Burkitt lymphoma, Chronic myeloid monocytic leukemia (CMML), Eosinphilia, Essential thrombocytosis, Hairy cell leukemia, and NK cell lymphoma.

The methods and compositions described herein can be used alone or in combination with other therapeutic agents and/or modalities. The term administered “in combination,” as used herein, is understood to mean that two (or more) different treatments are delivered to the subject during the course of the subject's affliction with the disorder, such that the effects of the treatments on the patient overlap at a point in time. In certain embodiments, the delivery of one treatment is still occurring when the delivery of the second begins, so that there is overlap in terms of administration. This is sometimes referred to herein as “simultaneous” or “concurrent delivery.” In other embodiments, the delivery of one treatment ends before the delivery of the other treatment begins. In certain embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In certain embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

In certain embodiments, a method or composition described herein, is administered in combination with one or more additional therapies, e.g., surgery, radiation therapy, or administration of another therapeutic preparation. In certain embodiments, the additional therapy may include chemotherapy, e.g., a cytotoxic agent. In certain embodiments the additional therapy may include a targeted therapy, e.g. a tyrosine kinase inhibitor, a proteasome inhibitor, or a protease inhibitor. In certain embodiments, the additional therapy may include an anti-inflammatory, anti-angiogenic, anti-fibrotic, or anti-proliferative compound, e.g., a steroid, a biologic immunomodulator, a monoclonal antibody, an antibody fragment, an aptamer, an siRNA, an antisense molecule, a fusion protein, a cytokine, a cytokine receptor, a bronchodialator, a statin, an anti-inflammatory agent (e.g. methotrexate), or an NSAID. In certain embodiments, the additional therapy may include a combination of therapeutics of different classes.

In certain embodiments, a method or composition described herein is administered in combination with a checkpoint inhibitor. The checkpoint inhibitor may, for example, be selected from a PD-1 antagonist, PD-L1 antagonist, CTLA-4 antagonist, adenosine A2A receptor antagonist, B7-H3 antagonist, B7-H4 antagonist, BTLA antagonist, KIR antagonist, LAG3 antagonist, TIM-3 antagonist, VISTA antagonist or TIGIT antagonist.

In certain embodiments, the checkpoint inhibitor is a PD-1 or PD-L1 inhibitor. PD-1 is a receptor present on the surface of T-cells that serves as an immune system checkpoint that inhibits or otherwise modulates T-cell activity at the appropriate time to prevent an overactive immune response. Cancer cells, however, can take advantage of this checkpoint by expressing ligands, for example, PD-L1, that interact with PD-1 on the surface of T-cells to shut down or modulate T-cell activity. Exemplary PD-1/PD-L1 based immune checkpoint inhibitors include antibody based therapeutics. Exemplary treatment methods that employ PD-1/PD-L1 based immune checkpoint inhibition are described in U.S. Pat. Nos. 8,728,474 and 9,073,994, and EP Patent No. 1537878B1, and, for example, include the use of anti-PD-1 antibodies. Exemplary anti-PD-1 antibodies are described, for example, in U.S. Pat. Nos. 8,952,136, 8,779,105, 8,008,449, 8,741,295, 9,205,148, 9,181,342, 9,102,728, 9,102,727, 8,952,136, 8,927,697, 8,900,587, 8,735,553, and 7,488,802. Exemplary anti-PD-1 antibodies include, for example, nivolumab (Opdivo®, Bristol-Myers Squibb Co.), pembrolizumab (Keytruda®, Merck Sharp & Dohme Corp.), PDR001 (Novartis Pharmaceuticals), and pidilizumab (CT-011, Cure Tech). Exemplary anti-PD-L1 antibodies are described, for example, in U.S. Pat. Nos. 9,273,135, 7,943,743, 9,175,082, 8,741,295, 8,552,154, and 8,217,149. Exemplary anti-PD-L1 antibodies include, for example, atezolizumab (Tecentriq®, Genentech), duvalumab (AstraZeneca), MEDI4736, avelumab, and BMS 936559 (Bristol Myers Squibb Co.).

In certain embodiments, a method or composition described herein is administered in combination with a CTLA-4 inhibitor. In the CTLA-4 pathway, the interaction of CTLA-4 on a T-cell with its ligands (e.g., CD80, also known as B7-1, and CD86) on the surface of an antigen presenting cells (rather than cancer cells) leads to T-cell inhibition. Exemplary CTLA-4 based immune checkpoint inhibition methods are described in U.S. Pat. Nos. 5,811,097, 5,855,887, 6,051,227. Exemplary anti-CTLA-4 antibodies are described in U.S. Pat. Nos. 6,984,720, 6,682,736, 7,311,910; 7,307,064, 7,109,003, 7,132,281, 6,207,156, 7,807,797, 7,824,679, 8,143,379, 8,263,073, 8,318,916, 8,017,114, 8,784,815, and 8,883,984, International (PCT) Publication Nos. WO98/42752, WO00/37504, and WO01/14424, and European Patent No. EP 1212422 Bi. Exemplary CTLA-4 antibodies include ipilimumab or tremelimumab.

Exemplary cytotoxic agents that can be administered in combination with a method or composition described herein include, for example, antimicrotubule agents, topoisomerase inhibitors, antimetabolites, protein synthesis and degradation inhibitors, mitotic inhibitors, alkylating agents, platinating agents, inhibitors of nucleic acid synthesis, histone deacetylase inhibitors (HDAC inhibitors, e.g., vorinostat (SAHA, MK0683), entinostat (MS-275), panobinostat (LBH589), trichostatin A (TSA), mocetinostat (MGCD0103), belinostat (PXD101), romidepsin (FK228, depsipeptide)), DNA methyltransferase inhibitors, nitrogen mustards, nitrosoureas, ethylenimines, alkyl sulfonates, triazenes, folate analogs, nucleoside analogs, ribonucleotide reductase inhibitors, vinca alkaloids, taxanes, epothilones, intercalating agents, agents capable of interfering with a signal transduction pathway, agents that promote apoptosis and radiation, or antibody molecule conjugates that bind surface proteins to deliver a toxic agent. In one embodiment, the cytotoxic agent that can be administered with a method or composition described herein is a platinum-based agent (such as cisplatin), cyclophosphamide, dacarbazine, methotrexate, fluorouracil, gemcitabine, capecitabine, hydroxyurea, topotecan, irinotecan, azacytidine, vorinostat, ixabepilone, bortezomib, taxanes (e.g., paclitaxel or docetaxel), cytochalasin B, gramicidin D, ethidium bromide, emetine, mitomycin, etoposide, tenoposide, vincristine, vinblastine, vinorelbine, colchicin, anthracyclines (e.g., doxorubicin or epirubicin) daunorubicin, dihydroxy anthracin dione, mitoxantrone, mithramycin, actinomycin D, adriamycin, 1-dehydrotestosterone, glucocorticoids, procaine, tetracaine, lidocaine, propranolol, puromycin, ricin, or maytansinoids.

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present invention, whether explicit or implicit herein. For example, where reference is made to a particular compound, that compound can be used in various embodiments of compositions of the present invention and/or in methods of the present invention, unless otherwise understood from the context. In other words, within this application, embodiments have been described and depicted in a way that enables a clear and concise application to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present invention also includes the specific quantitative value itself, unless specifically stated otherwise. As used herein, the term “about” refers to a ±10% variation from the nominal value unless otherwise indicated or inferred.

It should be understood that the order of steps or order for performing certain actions is immaterial so long as the present invention remain operable. Moreover, two or more steps or actions may be conducted simultaneously.

The use of any and all examples, or exemplary language herein, for example, “such as” or “including,” is intended merely to illustrate better the present invention and does not pose a limitation on the scope of the invention unless claimed. No language in the specification should be construed as indicating any non-claimed element as essential to the practice of the present invention.

### EXAMPLES

The following Examples are merely illustrative and are not intended to limit the scope or content of the invention in any way.

### Example 1

This example describes the construction of recombinant human sialidases (Neu1, Neu2, Neu3, and Neu 4).

The human sialidases Neu1, Neu2, Neu3 (isoform 1), and Neu4 (isoform 1) were expressed as secreted proteins with a 10×His tag (SEQ ID NO: 157). To express Neu1 as a secreted protein, the native N terminal signal peptide (MTGERPSTALPDRRWGPRILGFWGGCRVWVFAAIFLLLSLAASWSKA; SEQ ID NO: 80) was replaced by MDMRVPAQLLGLLLLWLPGARC (SEQ ID NO: 32), and the C terminal lysosomal signal motif (YGTL; SEQ ID NO: 33) was removed. To express Neu2, Neu3, and Neu4 as secreted proteins, the N terminal signal peptide MDMRVPAQLLGLLLLWLPGARC (SEQ ID NO: 32) was added to each.

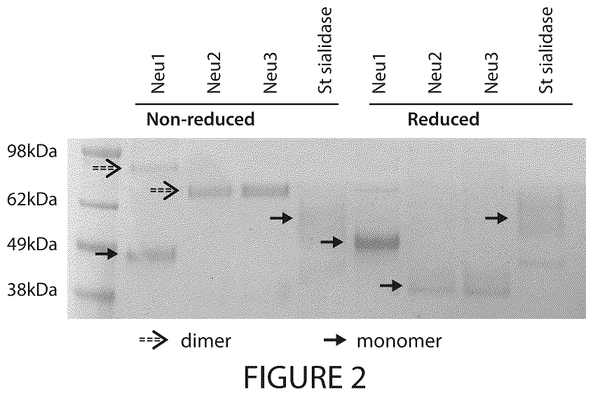

Sialidases were expressed in a 200 mL transfection of HEK293F human cells in 24-well plates using the pCEP4 mammalian expression vector. Sialidases were purified using Ni-NTA columns, quantified with a UV-Vis spectrophotometer (NanoDrop), and examined by SDS-PAGE as shown in FIG. 2. Neu1 expressed well, with a yield of ˜3 μg/ml, and was present primarily in a monomeric form. Neu2 and Neu3 expression each gave yields of ˜0.15 μg/mL and each were present primarily in a dimeric form. Neu4 had no detectable expression yield as measured by NanoDrop. Bacterial sialidase from Salmonella typhimurium (St-sialidase; SEQ ID NO: 7), which was used as a positive control for expression, gave a comparable yield to Neu1, and was present primarily in a monomeric form.

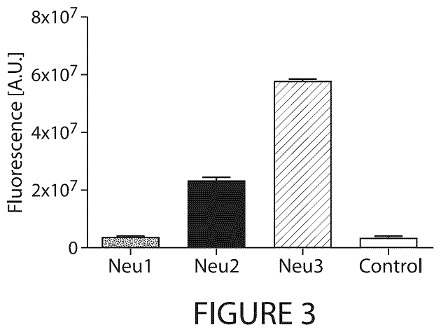

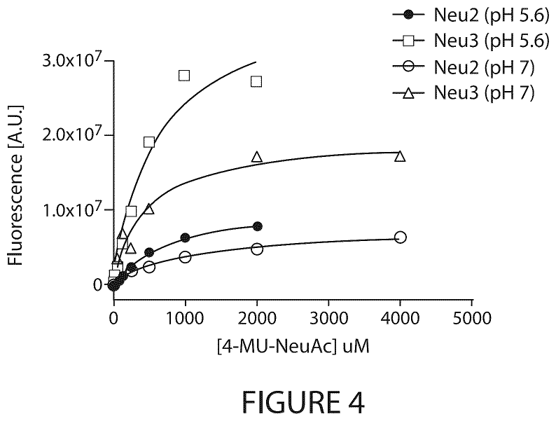

The activity of the recombinantly expressed sialidases was assayed by measuring the release of sialic acid from the fluorogenic substrate 4-methylumbelliferyl-N-acetylneuraminic acid (4MU-NeuAc). As shown in FIG. 3, Neu1 has no detectable activity above a no-enzyme control, which is consistent with previous reports indicating that Neu1 is inactive unless it is in complex with beta-galactosidase and protective protein/cathepsin A (PPCA). Neu2 and Neu3 were active. An enzyme kinetics assay was performed with Neu2 and Neu3. A fixed concentration of enzyme at 1 nM was incubated with fluorogenic substrate 4MU-NeuAc at concentrations ranging from 4000 μM to 7.8 μM. Assays were conducted at both acidic (pH 5.6) and neutral (pH 7) conditions. As shown in FIG. 4, both Neu2 and Neu3 were active at acidic and neutral conditions and showed enzyme kinetics that were comparable to those previously reported.

### Example 2

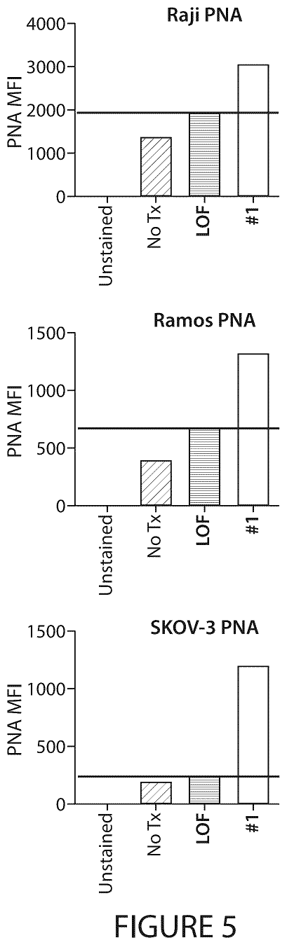

This example describes the degree of sialic acid removal from three human cancer target cell lines by neuraminidase constructs of the current invention. Specifically, Raji Burkitt lymphoma cells, Ramos lymphoma cells and SKOV-3 ovarian cells were treated for 16 hours using either neuraminidase construct #1 (a mutant Neu2-Fc protein having amino acid sequence SEQ ID NO: 149 encoded by nucleotide sequence SEQ ID NO: 156), a mutant neuraminidase lacking enzymatic activity (a similar mutant Neu2-Fc protein that also contains an E218A active site mutation that results in loss of neuraminidase function, “LOF”), or left untreated (No Tx). Cells were then stained with PNA, a lectin that binds to terminal galactose residues. An increase in PNA staining is indicative of the removal of terminal sialic acids by the neuraminidase and exposure of the underlying galactose. As seen in FIG. 5, all three cell lines demonstrated increased PNA staining to differing degrees by neuraminidase construct #1 as compared to LOF or No Tx. SKOV-3 experienced a high level of sialic acid removal, Ramos cells experienced an intermediate level of sialic acid removal and Raji cells experienced a lower level of sialic acid removal.

### Example 3

This example describes the effect of neuraminidase cotreatment on CAR-T cell engagement of target cells using neuraminidase constructs of the current invention. Specifically, the engagement by CD19 directed CAR-T engineered cells of Raji Burkitt lymphoma cells and Ramos lymphoma cells in the continued presence of neuraminidase for 16 hours was examined. Raji and Ramos cells, both of which are CD19 positive human cancer cells, were incubated with anti-CD19-ScFv-CD28-CD3zeta cells (ProMab Biotechnologies, Inc., Richmond, CA). The CAR-T cells and target cells were plated at the indicated E:T ratios in the presence of either neuraminidase construct #1 or LOF neuraminidase (100 μg/ml final concentration) with subsequent analysis of conditioned media following 16 hours incubation at 37° C. CAR-T cells were also plated out alone with no target cells (E:T ratio of 1:0). For all experiments the 1:0 ratio utilized 10,000 CAR-T cells and no target cells; the 1:1 ratio utilized 10,000 CAR-T cells and 10,000 target cells; the 1:2 ratio utilized 5,000 CAR-T cells and 10,000 target cells. Each condition was tested in quadruplicate.

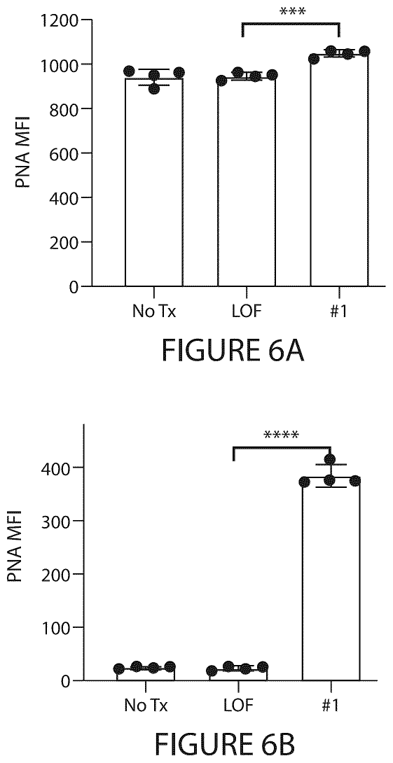

FIG. 6 depicts the degree of sialic acid removal from the CD19 positive Raji cells (FIG. 6A) and CD3 positive CAR-T cells (FIG. 6B) following 16 hours cotreatment with neuraminidase. Specifically, both target and effector cell were analyzed for PNA staining following 16 hours with either the LOF neuraminidase, neuraminidase construct #1 or untreated cells (No Tx). Neuraminidase construct #1 resulted in a significant increase in PNA staining in both Raji target cells and CAR-T cells indicating efficient removal of terminal sialic acids on the cell surface.

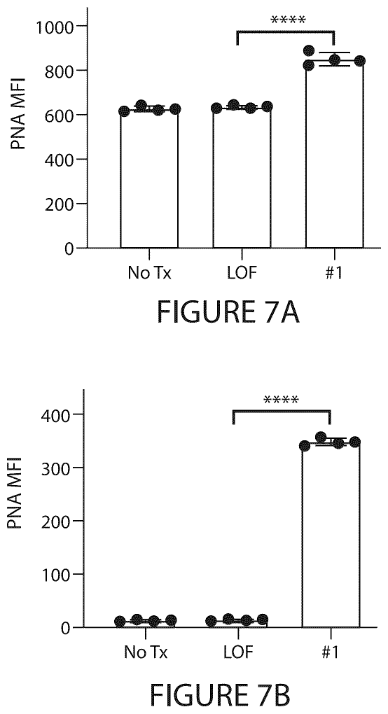

FIG. 7 depicts the degree of sialic acid removal from the CD19 positive Ramos cells (FIG. 7A) and CD3 positive CAR-T cells (FIG. 7B) following 16 hours cotreatment with neuraminidase construct #1. Specifically, both target and effector cells were analyzed for PNA staining following 16 hours with either the LOF neuraminidase, neuraminidase construct #1 or untreated cells (No Tx). Neuraminidase construct #1 resulted in a significant increase in PNA staining in both Ramos target cells and CAR-T cells indicating efficient removal of terminal sialic acids on the cell surface.

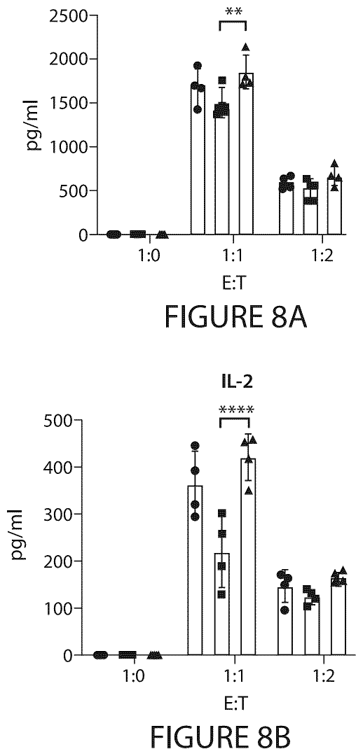

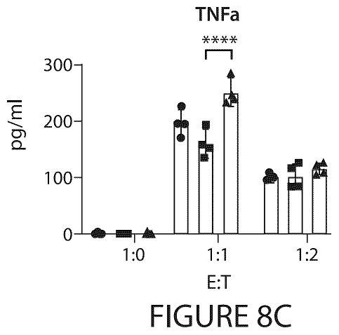

FIG. 8 depicts the analysis of secreted cytokines from the cells at the 16 hour time point. Specifically, following incubation of the CAR-T cells and the Raji cells with neuraminidase construct #1 for 16 hours, the cells were removed and the conditioned media analyzed for IFN-gamma, IL-2, IL-10, IL-6 and TNF-alpha by flow multiplex (LegendPlex). In each panel, the No Tx, LOF and neuraminidase construct #1 (respectively) analysis in quadruplicate assays at the indicated E:T ratio is shown. Neuraminidase treatment increased expression of IFN-gamma (FIG. 8A), IL-2 (FIG. 8B) and TNF-alpha (FIG. 8C) as compared to LOF or No Tx. No changes to IL-10 or IL-6 were observed between neuraminidase treatment as compared to LOF or No Tx.

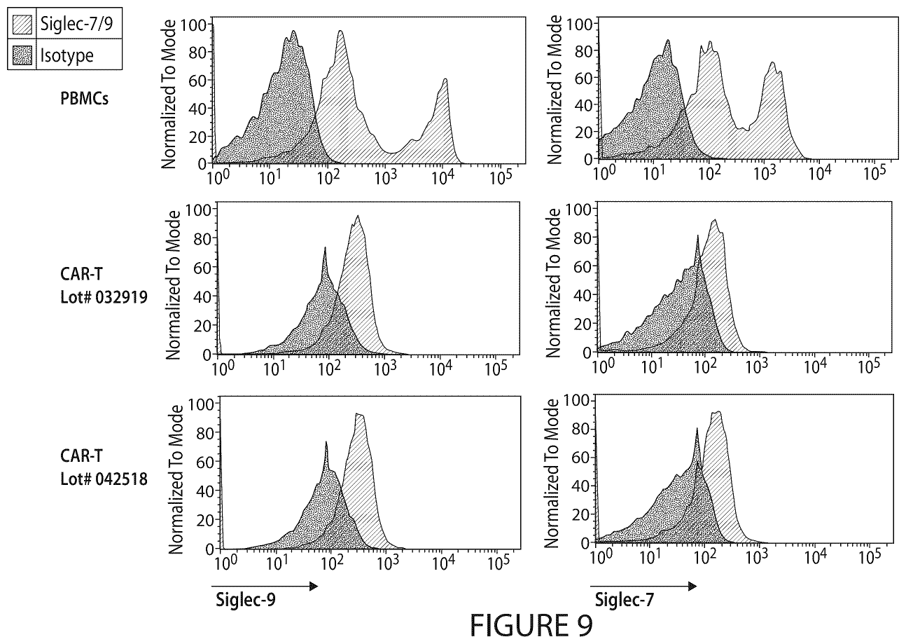

FIG. 9 depicts the analysis of Siglec7 and Siglec9 expression on CAR-T cells as compared to PBMCs used as a positive control. Specifically, CAR-T cells prepared from two independent donors (ProMab Biotechnologies Inc., Richmond, CA; Lot-032919 and Lot-042518) or PBMCs from a healthy donor were stained for Siglec7 expression (left panels) or for Siglec9 expression (right panels) and compared to isotype staining. For both CAR-T cell donor preparations, Siglec7 and Siglec9 specific, uniform staining was observed (light grey staining) versus isotype control (dark grey staining).

### Example 4

This example describes the testing of sialidases for their ability to increase the efficacy of chimeric antigen receptor (CAR) T-cell immunotherapies for solid malignancies.

To generate a DNA sequence encoding a tumor-specific CAR, a DNA sequence encoding a fully human single chain Fv specific for a tumor antigen (for example, m912 specific for mesothelin (Feng et al. (2009) MOL. CANCER THER. 8(5):1113-1118) is fused to a DNA encoding the CD8/CD3ζ, CD28/CD3ζ, or CD8/4-1BB/CD3ζ domain (see, for example, Zhong et al. (2010) Mol Ther. 18(2):413-420, or as available from ProMab Biotechnologies Inc., Richmond, CA). Alternatively, the CAR can target different tumor antigens such CD19, CD133, Her-2, EGFR, or VEGFR-2 that are highly over-expressed in tumors (using, for example, CARs as available from ProMab Biotechnologies Inc., Richmond, CA). Exemplary promoters for expression of a CAR construct include retroviral LTR, the SV40 promoter, or the human cytomegalovirus (CMV) promoter.

Exemplary DNA sequences encoding a sialidase include SEQ ID NO: 9, SEQ ID NO: 10, SEQ ID NO: 11, SEQ ID NO: 12, SEQ ID NO: 13, SEQ ID NO: 14, SEQ ID NO: 15, and SEQ ID NO: 16.

Exemplary promoters for expression of a sialidase construct include the PD-1 promoter, the NFAT promoter or an inducible promoter such as the tetracycline promoter. To express a sialidase as a secreted protein, an N terminal signal peptide (e.g., MDMRVPAQLLGLLLLWLPGARC; SEQ ID NO: 32) may be added or may replace a native N terminal signal peptide. A sialidase may be expressed alone, or with a tag, e.g., a human Fc tag. A sialidase may also be expressed as an antibody-sialidase genetic fusion protein, or as an antibody sialidase conjugate (ASC) containing the fusion protein.

The architecture for three exemplary ASCs is depicted in FIG. 1. The first type of ASC, referred to as “Raptor,” includes an antibody (with two heavy chains and two light chains) with a sialidase fused at the C-terminus of each heavy chain of the antibody. The second type of ASC, referred to as “Janus,” contains one antibody arm (with one heavy chain and one light chain), and one sialidase-Fc fusion with a sialidase fused at the N-terminus of the Fc. Each Fc domain polypeptide in the Janus ASC contains either the “knob” (T366Y) or “hole” (Y407T) mutation for heterodimerization (residue numbers according to EU numbering, Kabat, E. A., et al. (1991) supra). The third type of ASC, referred to as “Lobster,” contains two Fc domain polypeptides each with a sialidase fused at the N-terminus of the Fc and a scFv fused at the C-terminus of the Fc. An exemplary Lobster ASC (amino acid sequence SEQ ID NO: 43, encoded by nucleic acid sequence SEQ ID NO: 55) includes a mutant human Neu2 (including a deletion of M1 and V6Y and I187K substitutions) and a scFv derived from trastuzumab.

The CAR sequence and sialidase sequence are inserted into the same or separate γ-retroviral vectors. The CAR- and sialidase-encoding plasmids are then transfected into 293T H29 and 293VecRD114 packaging cell lines to produce a retrovirus, as previously described (Hollyman et al. (2009) J. IMMUNOTHER. 32(2):169-180).

Peripheral blood mononuclear cells (PBMCs) are isolated by low-density centrifugation on Lymphoprep (Stem Cell Technology) and activated with phytohemagglutinin (2 μg/ml; Remel). Two days after isolation, PBMCs are transduced with 293VecRD114-produced retroviral particles encoding the CAR and/or sialidase and spinoculated for 1 hour at 1,800×g on plates coated with retronectin (15 g/ml; r-Fibronectin, Takara). Transduced PBMCs are maintained in IL-2 (20 UI/ml; Novartis). Pure populations of CD4+, CD8+, CAR+, and/or sialidase+ T cells are obtained by flow cytometry-based sorting.

An orthotopic mouse model of pleural mesothelioma using female NOD/SCID7 mice (The Jackson Laboratory) aged 4 to 6 weeks is used. Alternatively, NSG/NOG xenograft survival and imaging studies can be used to characterize CAR-T cell function in vivo. Mice are anesthetized using inhaled isoflurane and oxygen, with bupivacaine administered for analgesia. Direct intrapleural injection of 1×10⁵ to 1×10⁶ tumor cells in 200 μl of serum-free medium via a right thoracic incision is performed to establish orthotopic tumors. Additional tumor models include those using mouse ovarian tumor lines.

Tumor-bearing mice are treated with or without 4×10⁴ to 1×10⁵ transduced T cells (in 200 μl of serum-free medium) adoptively transferred into the thoracic cavity by direct intrapleural injection. Additionally, tumor-bearing mice are treated with or without a sialidase, injected intraperitoneally at, for example, 10 mg/kg every 5 days. Tumor growth is monitored and quantified in vivo by bioluminescence imaging.

Comparison is made of mice bearing tumors alone, mice treated with T cells engineered to express the CAR alone and mice treated with T cells engineered to express CAR and the sialidase. Alternatively, comparison is made of mice bearing tumors alone, mice treated with T cells engineered to express the CAR alone and mice treated with T cells engineered to express CAR and a sialidase.

### INCORPORATION BY REFERENCE

The entire disclosure of each of the patent and scientific documents referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

1. An isolated immune cell modified to have increased sialidase activity relative to a similar or identical cell that has not been modified.

2. (canceled)

3. The isolated immune cell of claim 1, wherein the sialidase is a eukaryotic sialidase.

4. The isolated immune cell of claim 3, wherein the sialidase is a mammalian sialidase.

5. (canceled)

6. The isolated immune cell of claim 4, wherein the sialidase is a human sialidase selected from Neu1, Neu2, Neu3 and Neu4.

7. The isolated immune cell of claim 4, wherein the sialidase comprises an amino acid sequence selected from SEQ ID NO: 1, SEQ ID NO: 2, SEQ ID NO: 3, SEQ ID NO: 4, SEQ ID NO: 5, and SEQ ID NO: 6.

8. The isolated immune cell of claim 1, wherein the sialidase is a prokaryotic sialidase.

9. The isolated immune cell of claim 8, wherein the sialidase is selected from a Salmonella typhimurium sialidase and a Vibrio cholera sialidase.

10. The isolated immune cell of claim 8, wherein the sialidase comprises an amino acid sequence selected from SEQ ID NO: 7 and SEQ ID NO: 8.

11. (canceled)

12. The isolated immune cell of claim 1, wherein the sialidase is a mutant sialidase.

13. The isolated immune cell of claim 12, wherein the mutant sialidase comprises an amino acid sequence selected from SEQ ID NO: 36, SEQ ID NO: 37, SEQ ID NO: 38, SEQ ID NO: 39, SEQ ID NO: 63, SEQ ID NO: 64, SEQ ID NO: 65, SEQ ID NO: 66, SEQ ID NO: 83, SEQ ID NO: 84, SEQ ID NO: 85, SEQ ID NO: 86, SEQ ID NO: 87, SEQ ID NO: 88, and SEQ ID NO: 89.

14. (canceled)

15. The isolated immune cell of claim 1, wherein the immune cell is selected from a T-cell, a Tumor Infiltrating Lymphocyte (TIL), and a natural killer (NK) cell.

16. (canceled)

17. The isolated immune cell of claim 1, wherein the immune cell further comprises an exogenous nucleotide sequence encoding a chimeric antigen receptor (CAR).

18. The isolated immune cell of claim 17, wherein the CAR binds a cancer antigen.

19. The isolated immune cell of claim 18, wherein the cancer antigen is selected from mesothelin, prostate specific membrane antigen (PSMA), prostate stem cell antigen (PCSA), carbonic anhydrase IX (CAIX), carcinoembryonic antigen (CEA), CD5, CD7, CD10, CD19, CD20, CD22, CD30, CD33, CD34, CD38, CD41, CD44, CD47, CD49f, CD56, CD74, CD123, CD133, CD138, epithelial glycoprotein2 (EGP 2), epithelial glycoprotein-40 (EGP-40), epithelial cell adhesion molecule (EpCAM), folate-binding protein (FBP), fetal acetylcholine receptor (AChR), folate receptor-α and β (FRa and β), Ganglioside G2 (GD2), Ganglioside G3 (GD3), an Epidermal Growth Factor Receptor (EGFR), Epidermal Growth Factor Receptor 2 (HER-2/ERB2), Epidermal Growth Factor Receptor vIII (EGFRvIII), ERB3, ERB4, human telomerase reverse transcriptase (hTERT), Interleukin-13 receptor subunit alpha-2 (IL-13Ra2), K-light chain, kinase insert domain receptor (KDR), Lewis A (CA19.9), Lewis Y (LeY), LI cell adhesion molecule (LICAM), melanoma-associated antigen 1 (melanoma antigen family A1, MAGE-A1), Mucin 16 (MUC-16), Mucin 1 (MUC-1), KG2D ligands, cancer-testis antigen NY-ESO-1, oncofetal antigen (h5T4), tumor-associated glycoprotein 72 (TAG-72), vascular endothelial growth factor R2 (VEGF-R2), Wilms tumor protein (WT-1), type 1 tyrosine-protein kinase transmembrane receptor (ROR1), B7-H3 (CD276), B7-H6 (Nkp30), Chondroitin sulfate proteoglycan-4 (CSPG4), DNAX Accessory Molecule (DNAM-1), Ephrin type A Receptor 2 (EpHA2), Fibroblast Associated Protein (FAP), Gp100/HLA-A2, Glypican 3 (GPC3), HA-IH, HERK-V, IL-1 IRa, Latent Membrane Protein 1 (LMP1), Neural cell-adhesion molecule (N-CAM/CD56), programmed cell death receptor ligand 1 (PD-L1), B Cell Maturation Antigen (BCMA), and Trail Receptor (TRAIL R).

20. (canceled)

21. A pharmaceutical composition comprising the isolated immune cell of claim 1, and a pharmaceutically acceptable carrier or diluent.

22. A pharmaceutical composition comprising:
(a) an isolated immune cell;
(b) a sialidase; and
(c) a pharmaceutically acceptable carrier or diluent.

23.-42. (canceled)

43. A method of treating cancer in a subject in need thereof, the method comprising administering to the subject an effective amount of the isolated immune cell of claim 1, thereby to treat the cancer in the subject.

44. (canceled)

45. A method of treating cancer in a subject in need thereof, the method comprising administering to the subject an effective amount of the pharmaceutical composition of claim 21, thereby to treat the cancer in the subject.

46. A method of treating cancer in a subject in need thereof, the method comprising administering to the subject an isolated immune cell and a sialidase, thereby to treat the cancer in the subject.

47.-66. (canceled)

67. The method of claim 46, wherein the isolated immune cell and the sialidase are administered separately or in combination.

68. The method of claim 46, wherein the isolated immune cell and the sialidase are administered at the same time.

69.-81. (canceled)
